(19) United States
(12) Patent Application Publication
(10) Pub. No.: US 2002/0081592 A1
(43) Pub. Date: Jun. 27, 2002

(54) Reproduction-specific genes

(75) Inventors: Peijing Jeremy Wang, Cambridge, MA (US); David C. Page, Winchester, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA (US)

(21) Appl. No.: 09/801,574

(22) Filed: Mar. 7, 2001

Related U.S. Application Data
(60) Provisional application No. 60/187,518, filed on Mar. 7, 2000.
(60) Provisional application No. 60/261,557, filed on Jan. 12, 2001.

(51) Int. Cl. C12Q 1/68; C07H 21/04; C12N 9/64; C12P 21/02; C12N 5/06

(52) U.S. Cl. 435/6; 435/325; 435/320.1; 435/69.1; 435/226; 536/23.2

(57) ABSTRACT

Reproduction-specific nucleic acid molecules, particularly those that are indicative of or associated with infertility in men, proteins encoded by these reproduction-specific nucleic acid molecules and antibodies that bind such proteins are described. Also described are variant reproduction-specific genes and proteins, and antibodies which bind such proteins, as well as methods of using the reproduction-specific genes, proteins and antibodies and methods of using the variant reproduction-specific genes, proteins and antibodies.

### RELATED APPLICATION

This application claims the benefit of U.S. provisional application Serial No. 60/187,518, filed on Mar. 7, 2000, and U.S. provisional application Serial No. 60/261,557, filed on Jan. 12, 2001. The entire teachings of the above applications are incorporated herein by reference.

### BACKGROUND OF THE INVENTION

Infertility is of great clinical significance, and between 2 and 7% of couples are infertile. Both physical and genetic factors are associated with male infertility. Some genetic factors are chromosomal aberrations, including: chromosomal translocations, Down's syndrome, Klinefilter's syndrome and Y chromosome microdeletions. Many cases of azoospermia are idiopathic (have no obvious cause) in that the subject is infertile but otherwise healthy. Previous research has suggested that genetic factors are important contributors to these cases, but these factors have not been identified.

### SUMMARY OF THE INVENTION

Spermatogonial stem cells are designated as undifferentiated spermatogonia; they are capable of self-renewal and persist as a constant population in adults. While renewing themselves, some of these stem cells begin to differentiate to give rise to type A spermatogonia. Type A spermatogonia divide four times and differentiate to eventually become type B spermatogonia. Type B spermatogonia divide once, enter meiosis at puberty, and eventually become mature sperm.

Described herein are novel nucleic acid molecules, referred to as reproduction-specific nucleic acid molecules, from spernatogonia (the stem cells of male germ cells); novel reproduction-specific proteins; antibodies that bind the proteins; and uses of the nucleic acid molecules or portions thereof, proteins and antibodies. The novel nucleic acid molecules of the present invention fall into three classes: 1) male germ cell-specific nucleic acid molecules, which are nucleic acid molecules that are expressed only in male germ cells; 2) testis-specific nucleic acid molecules, which are nucleic acid molecules that are expressed only in testis; and 3) testis- and ovary-specific nucleic acid molecules, which are nucleic acid molecules that are only expressed in testis and ovary. As further described herein, the present work has resulted in identification of a number of variants of the testis-specific genes, TAF2Q and TEX11 which are present on sex chromosome X.

The present invention also relates to variant forms of reproduction-specific nucleic acid molecules (referred to as variant reproduction-specific nucleic acid molecules) that are indicative of or associated with infertility in men, proteins encoded by variant reproduction-specific nucleic acid molecules (referred to as variant reproduction-specific proteins), antibodies that bind such proteins, and methods of using the variant reproduction-specific nucleic acid molecules or portions thereof, proteins encoded by variant reproduction-specific nucleic acid molecules, and antibodies that bind variant reproduction-specific proteins.

The present invention encompasses all of these nucleic acid molecules, their complements, portions of the nucleic acid molecules and their complements, and any nucleic acid molecules that, through the degeneracy of the genetic code, encode a protein whose sequence is presented herein or a protein encoded by nucleic acid molecules whose sequence is specifically presented herein. Nucleic acid molecules of the present invention (genes, genomic sequences, cDNAs and portions of the foregoing) are useful, for example, as hybridization probes and as primers for amplification methods which, in turn, are useful in methods of detecting the presence, absence or alteration of the nucleic acid molecules described herein.

The present invention also relates to methods of identifying or determining differences in one or more of these reproduction-specific nucleic acid molecules that are associated with (indicative of) infertility in men. For example, nucleic acid molecules from tissues or body fluids, such as nucleic acid molecules in blood, obtained from one or more males with a known condition, such as lack of sperm production or reduced sperm count, can be assessed, using the nucleic acid molecule(s) described herein, or characteristic portions thereof, to determine whether the male(s) lacks some or all of the nucleic acid molecule(s) described herein or has a variant nucleic acid molecule(s) (e.g., in which there is a deletion, substitution, addition or mutation, compared to the sequences presented herein). Nucleic acid molecules (e.g., from a male with reduced sperm count or viability) can be assessed, using nucleic acid molecules described herein or nucleic acid molecules which hybridize to a nucleic acid molecule described herein, to determine whether they are associated with or causative for infertility (e.g., reduced sperm count or viability). For example, the presence or absence of all or a portion of a nucleic acid molecule or nucleic acid molecules shown to be necessary for fertility or adequate sperm count can be assessed, using nucleic acid molecules which hybridize to the nucleic acid molecule or nucleic acid molecules of interest to determine the basis for an individual's infertility or reduced sperm count. In one embodiment, the occurrence of one or more reproduction-specific nucleic acid molecules or a characteristic portion of one or more reproduction-specific nucleic acid molecules is assessed in a sample containing nucleic acid molecules.

In another embodiment, deletion or alteration of one of the nucleic acid molecules described herein or a characteristic portion thereof is used to assess a nucleic acid sample obtained from a male who has a reduced sperm count or spermatogenic failure. Lack of hybridization of reproduction-specific nucleic acid molecules known to be present in fertile men, but not in infertile men, to nucleic acid molecules in the sample (sample nucleic acid molecules) indicates that the gene is not present in the sample nucleic acid molecules or is present in a variant form which does not hybridize to reproduction-specific nucleic acid molecules present in fertile men. In the present methods, sample nucleic acid molecule can be analyzed for the alteration or occurrence of one or more of the reproduction-specific nucleic acid molecules and can be analyzed for one or more of the three classes of nucleic acid molecules described herein. For example, a group of nucleic acid molecule probes (sequences) can be used to analyze sample nucleic acid molecule; the set of probes can include nucleic acid molecule probes which hybridize to two or more reproduction-specific nucleic acid molecules or nucleic acid molecule probes which hybridize only to variant nucleic acid molecules characteristic of (indicative of) infertility in men.

Nucleic acid molecules described herein are also useful as primers in an amplification method, such as PCR, useful for identifying and amplifying reproduction-specific nucleic acid molecules in a sample (e.g., blood). Further, proteins or peptides encoded by a reproduction-specific nucleic acid molecule can be assessed in samples. This can be carried out, for example, using antibodies which recognize proteins or peptides of the present invention (proteins or peptides encoded by nucleic acid molecules described herein or a variant thereof that is present in infertile men, but not in fertile men or vice versa).

The present invention also relates to methods of diagnosing or aiding in the diagnosis of infertility in men, based on differences present in at least one of these nucleic acid molecules (between infertile men and fertile men). For example, one embodiment of this invention is a diagnostic method, such as a method of determining whether nucleic acid molecules from a man (e.g., obtained from blood, other tissue) contain at least one nucleic acid molecule which varies (comprises a substitution, deletion, addition or rearrangement) from reproduction-specific nucleic acid molecules in a manner shown to be indicative of or characteristic of infertility

The present invention further relates to proteins disclosed herein or encoded by nucleic acid molecules described herein, portions of the proteins (such as characteristic portions, referred to as characteristic peptides, useful in distinguishing between infertile and fertile men) and antibodies (monoclonal or polyclonal) that bind proteins of the present invention or characteristic portions thereof. The proteins of the present invention include proteins encoded by nucleic acid molecules whose sequence is disclosed herein; proteins whose amino acid sequences are disclosed herein; and proteins whose amino acid sequence differs from the amino acid sequence of proteins disclosed herein by at least one (one or more) residue and are associated with or indicative of azoospermia (lack of or reduction in sperm production), referred to as variant reproduction-specific proteins. Antibodies of the present invention are useful in methods of diagnosing or aiding in the diagnosis of infertility in men.

A further subject of the present invention is a method of contraception in which sperm production and/or function are altered, preferably reversibly. In the method, the function of one or more of the nucleic acid molecules or one or more of the proteins described herein is disrupted in a man, with the result that sperm production does not occur; occurs only to a limited extent (an extent less than normally occurs in the individual); or is otherwise altered (e.g., defective sperm, such as sperm with decreased motility or shortened lifespan, are produced). For example, a reproduction-specific gene shown to be present in fertile men, but not in infertile men, is targeted and its function (expression) is disrupted, with the result that the gene is not expressed, is expressed at a reduced level (at a level lower than if it the gene function had not been disrupted) or, when it is expressed, the resulting product is defective. Alternatively, a protein or proteins encoded by a reproduction-cell specific gene(s) is targeted and its function is disrupted and/or the protein is broken down (e.g., by proteolysis). Agents (drugs) useful in the method are also the subject of the present invention.

Further, the present invention relates to a method of treating reduced sperm count, reduced sperm function, reduced sperm motility or spermatogenic failure. In one embodiment, reduced sperm count is increased by administering an agent that enhances the activity, of a reproduction-specific gene or genes. Preferably, such drugs target (act essentially exclusively upon) a reproduction-specific gene or portion thereof. Such drugs can be administered by a variety of routes, such as oral or intravenous administration. In another embodiment, a gene therapy method is used. For example, a one or more nucleic acid molecule(s) described herein, or a portion thereof which encodes a functional protein, is introduced into a man whose sperm count is reduced and in whom the nucleic acid molecule is expressed, and the resulting protein replaces or supplements the protein normally produced or enhances the quantity produced.

The nucleic acid molecules, proteins and antibodies that bind proteins of the present invention, or portions thereof, are also useful as markers for spermatogonial cells.

As described herein, particular variants of the testis-specific X-linked TAF2Q and TEX11 nucleic acid molecules from infertile men were identified by methods described herein. These variants result from alternation in the nucleic acid molecule; some nucleic acid molecules alterations are silent (do not result in a change in amino acid), while others result in an amino acid alteration or in truncation of the encoded protein. These variants are associated with male infertility. The particular variants are useful in the methods described herein and are shown in FIGS. 107, 108, 111 and 112.

Thus, the invention relates to an isolated reproduction-specific nucleic acid molecule comprising a nucleic acid molecule having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and the complements thereof.

The invention also relates to an isolated reproduction-specific nucleic acid molecule comprising a portion of a nucleic acid molecule having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and the complements thereof, wherein said portion is at least 14 contiguous nucleotides in length.

The invention further relates to an isolated reproduction-specific nucleic acid molecule comprising a nucleic acid molecule which hybridizes under high stringency hybridization conditions to a nucleic acid molecule having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and the complements thereof.

The invention also relates to an isolated reproduction-specific nucleic acid molecule comprising a nucleic acid molecule having a nucleotide sequence which is at least 70% identical to a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and the complements thereof.

The invention further relates to an isolated reproduction-specific nucleic acid molecule which encodes a protein having an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 51, 53, 57, 60, 63, 65, 68, 70, 72, 76, 78, 80, 82, 84, 85, 88, and90.

The invention further relates to an isolated variant reproduction-specific nucleic acid molecule comprising a nucleic acid molecule having the nucleic acid sequence of SEQ ID NO: 89 having one or more alterations selected from the group consisting of A320G, T325A, C381T, G400A, A491G, G1282A, C1449A, T2219C, A2250T, T2295C and T2472C. The invention also relates to an isolated variant reproduction-specific nucleic acid molecule comprising a nucleic acid molecule having the nucleic acid sequence of SEQ ID NO: 50 having one or more alterations selected from the group consisting of the alterations shown in FIG. 112.

The invention also relates to nucleic acid constructs comprising an isolated reproduction-specific nucleic acid molecule according to the invention operably linked to at least one regulatory sequence, and to a host cell comprising such nucleic acid constructs.

The invention also relates to an isolated protein comprising an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 51, 53, 57, 60, 63, 65, 68, 70, 72, 76, 78, 80, 82, 84, 85, 88, and 90. The invention also pertains to an isolated protein comprising a portion of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 51, 53, 57, 60, 63, 65, 68, 70, 72, 76, 78, 80, 82, 84, 85, 88, and 90, wherein said portion is at least 7 contiguous amino acids. The invention is also drawn to an isolated protein comprising the amino acid sequence of SEQ ID NO: 90 having one or more alterations selected from the group consisting of W109R, V1341, G164R, N483K and V740A. The invention also relates to an isolated protein encoded by a nucleic acid molecule according to the invention. The invention further relates to an antibody which specifically binds a protein according to the invention.

The invention also relates to a method of diagnosing infertility associated with alteration of a gene having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89, and whose alteration is associated with infertility, comprising the steps of: (a) obtaining a DNA sample to be assessed; (b) processing the DNA sample such that the DNA is available for hybridization; (c) combining the DNA of step (b) with nucleotide sequences complementary to the altered nucleotide sequence of said gene, whose alteration is associated with infertility, under conditions appropriate for hybridization of the probes with complementary nucleotide sequences in the DNA sample, thereby producing a combination; and (d) detecting hybridization in the combination, wherein presence of hybridization in the combination is indicative of infertility associated with an alteration of said gene.

The invention also relates to a method of diagnosing infertility associated with alteration of a gene having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89, and whose alteration is associated with infertility, comprising the steps of: (a) obtaining a DNA sample to be assessed; (b) processing the DNA sample such that the DNA is available for hybridization; (c) combining the DNA of step (b) with nucleotide sequences complementary to the nucleotide sequence of said gene, whose alteration is associated with infertility, under conditions appropriate for hybridization of the probes with complementary nucleotide sequences in the DNA sample, thereby producing a combination; and (d) detecting hybridization in the combination, wherein absence of hybridization in the combination is indicative of infertility associated with an alteration of said gene. In a preferred embodiment, infertility is a result of reduced sperm count, reduced sperm motility, malformed sperm, or combinations thereof.

### BRIEF DESCRIPTION OF THE DRAWINGS

FIG. 1 shows the Spg1 cDNA sequence.

FIG. 2 shows the Spg1 encoded protein sequence.

FIGS. 3a-3c show the Spg2 cDNA sequence.

FIG. 4 shows the Spg2 encoded protein sequence.

FIGS. 5a-5b show the Spg3 cDNA sequence.

FIG. 6 shows the Spg3 encoded protein sequence.

FIGS. 7a-7d show the Spg5 cDNA sequence.

FIGS. 8a-8b show the Spg5 encoded protein sequence.

FIGS. 9a-9b show the Spg13 cDNA sequence.

FIG. 10 shows the Spg13 encoded protein sequence.

FIGS. 11a-11b show the Spg14 cDNA sequence.

FIGS. 12a-12b show the Spg14 encoded protein sequence.

FIGS. 13a-13b show the Spg15 cDNA sequence.

FIGS. 14a-14b show the Spg15 encoded protein sequence.

FIGS. 15a-15b show the Spg16 cDNA sequence.

FIG. 16 shows the Spg16 encoded protein sequence.

FIGS. 17a-17b show the Spg17 cDNA sequence.

FIG. 18 shows the Spg17 encoded protein sequence.

FIG. 19 shows the Spg18 CDNA sequence

FIG. 20 shows the Spg18 encoded protein sequence.

FIGS. 21a-21b show the Spg25 cDNA sequence.

FIGS. 22a-22b show the Spg25 encoded protein sequence.

FIG. 23 shows the Spg27 cDNA sequence.

FIG. 24 shows the Spg27 encoded protein sequence.

FIGS. 25a-25b show the Spg33 cDNA sequence.

FIG. 26 shows the Spg33 encoded protein sequence.

FIG. 27 shows the Spg34 cDNA sequence.

FIG. 28 shows the Spg34 encoded protein sequence.

FIGS. 29a-29b show the Spg39 cDNA sequence.

FIG. 30 shows the Spg39 encoded protein sequence.

FIGS. 31a-31b show the Spg46 cDNA sequence.

FIGS. 32a-32b show the Spg46 encoded protein sequence.

FIGS. 33a-33b show the Spg58 cDNA sequence.

FIGS. 34a-34b show the Spg58 encoded protein sequence.

FIG. 35 shows the Spg59 cDNA sequence.

FIG. 36 shows the Spg59 encoded protein sequence

FIGS. 37a-37b show the Spg64 cDNA sequence.

FIG. 38 shows the Spg64 encoded protein sequence.

FIGS. 39a-39b show the Spg65 cDNA sequence.

FIG. 40 shows the Spg65 encoded protein sequence.

FIGS. 41a-41b show the Spg69 cDNA sequence.

FIG. 42 shows the Spg69 encoded protein sequence.

FIGS. 43a-43b show the Spg70 cDNA sequence.

FIG. 44 shows the Spg70 encoded protein sequence.

FIGS. 45a-45c show the Spg85 cDNA sequence.

FIG. 46 shows the Spg85 encoded protein sequence.

FIGS. 47a-47b show the Spg87 cDNA sequence.

FIG. 48 shows the Spg87 encoded protein sequence.

FIGS. 49 shows the Spg84 cDNA sequence.

FIG. 50 shows the hSPG1 cDNA sequence.

FIG. 51 shows the hSPG1 encoded protein sequence.

FIGS. 52a-52b show the hSPG3a cDNA sequence.

FIG. 53 shows the hSPG3a encoded protein sequence.

FIGS. 54a-54e show the hSPG3a genomic DNA sequence.

FIG. 55 shows the hSPG3b cDNA sequence.

FIGS. 56a-56d show the hSPG5 cDNA sequence.

FIGS. 57a-57b show the hSPG5 encoded protein sequence.

FIGS. 58a-58e show the hSPG5 genomic DNA sequence.

FIGS. 59a-59c show the hSPG15 cDNA sequence.

FIG. 60 shows the hSPG15 encoded protein sequence.

FIGS. 61a-61t show the hSPG15 genomic DNA sequence.

FIG. 62 shows the hSPG18 cDNA sequence.

FIGS. 63a-63b show the hSPG18 encoded protein sequence.

FIGS. 64a-64b show the hSPG25 CDNA sequence.

FIG. 65 shows the hSPG25 encoded protein sequence.

FIG. 66 shows the hSPG27 cDNA sequence.

FIGS. 67a-67b show the hSPG34a cDNA sequence.

FIG. 68 shows the hSPG34a encoded protein sequence.

FIG. 69 shows the hSPG34b cDNA sequence.

FIG. 70 shows the hSPG34b encoded protein sequence.

FIGS. 71a-71b show the hSPG39a cDNA sequence.

FIG. 72 shows the hSPG39a encoded protein sequence.

FIG. 73a and 73b show the hSPG39a genomic DNA sequence.

FIG. 74 shows the hSPG39b cDNA sequence.

FIGS. 75a-75b show the hSPG46 cDNA sequence.

FIGS. 76a-76b show the hSPG46 encoded protein sequence.

FIGS. 77 shows the hSPG64 cDNA sequence.

FIGS. 78a-78b show the hSPG64 encoded protein sequence.

FIGS. 79a-79b show the hSPG85 cDNA sequence.

FIG. 80 shows the hSPG85 encoded protein sequence.

FIGS. 81a-81b show the hSPG13 cDNA long form sequence.

FIG. 82 shows the sequence of the protein encoded by hSPG13 long form.

FIGS. 83a-83b show is the hSPG13 cDNA short form sequence.

FIG. 84 shows the sequence of the protein encoded by hSPG13 short form.

FIG. 85 shows the hSPG39b encoded protein sequence.

FIGS. 86a-86b show the hSPG39b genomic DNA sequence.

FIGS. 87a-87b show the hSPG70 cDNA sequence.

FIG. 88 shows the hSPG70 encoded protein sequence.

FIGS. 89a and 89b show the nucleic acid sequence of TEX1 (SEQ ID NO: 89).

FIG. 90 shows the amino acid sequence of TEX11 (SEQ ID NO: 90).

FIG. 91 depicts the identification of spermatogonia-specific genes by cDNA subtraction.

FIG. 92 depicts the known germ cell-specific genes enriched by subtraction.

FIG. 93 depicts the genes identified by the subtraction.

FIG. 94 depicts the novel mouse germ cell specific genes identified by subtraction.

FIG. 95 depicts the post-transcriptional gene regulation of germ cell development.

FIG. 96 depicts the abundance of male germ-cell-specific genes on X Chromosome.

FIG. 97 depicts the rapid evolution of spermatogonia-specific genes in mouse and human.

FIG. 98 depicts hybrid male sterility in mice.

FIG. 99 depicts candidate genes for Hst-3.

FIG. 100 depicts the 14 novel human testis-specific genes.

FIG. 101 depicts the BAC physical map and gene structure of TEX11.

FIG. 102 depicts the high throughput mutation screening by genomic sequencing.

FIG. 103 depicts the mutations found in infertile but not fertile males

FIG. 104 depicts the clustering of mutations in 3′ but not 5′ regions of introns of TEX11.

FIG. 105 depicts the epigenetic down regulation of X-linked genes during male meiosis.

FIG. 106 depicts the abundance of spermatogonia genes on the X Chromosomes.

FIG. 107 depicts the intronic variants in TEX11.

FIG. 108 depicts the coding variants in TEX11.

FIG. 109 is a pedigree chart of WHT3759 depicting infertility as a result of mutations in TEX11.

FIG. 110 depicts the coding variants found in infertile but not fertile males.

FIG. 111 is a pedigree chart of WVT2508 depicting a mutation in TAF2Q resulting in infertility.

FIG. 112 depicts the variants in TAF2Q.

FIGS. 113a, 113b and 113c depict the twenty-three spermatogonially expressed, germ cell specific genes in mouse and their humun orthologs.

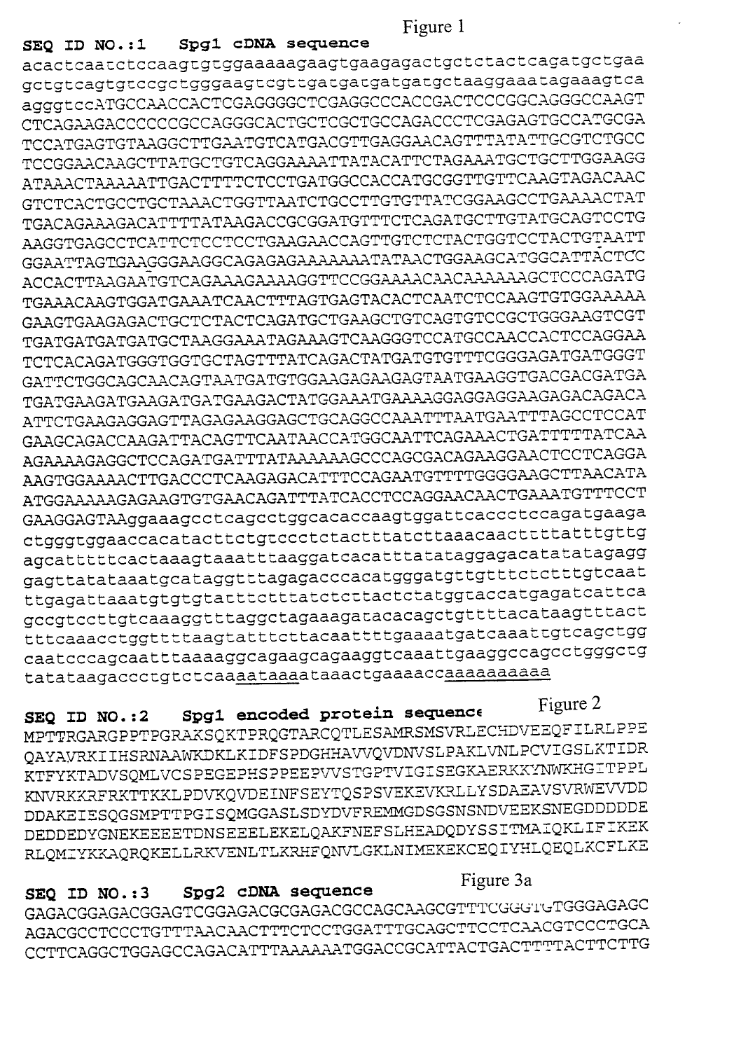

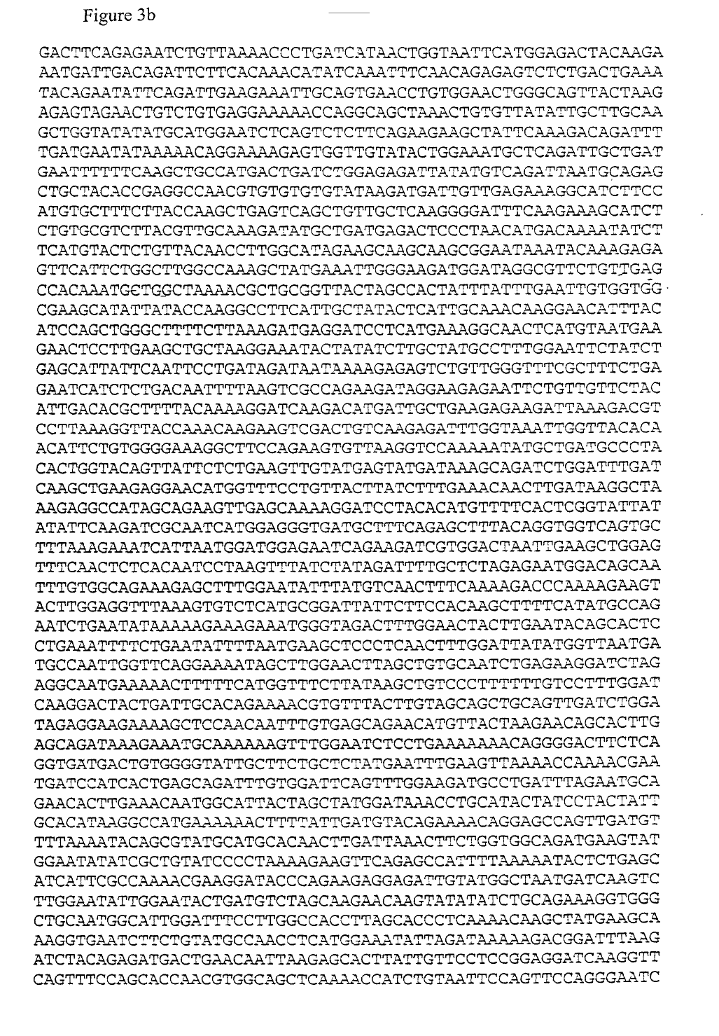

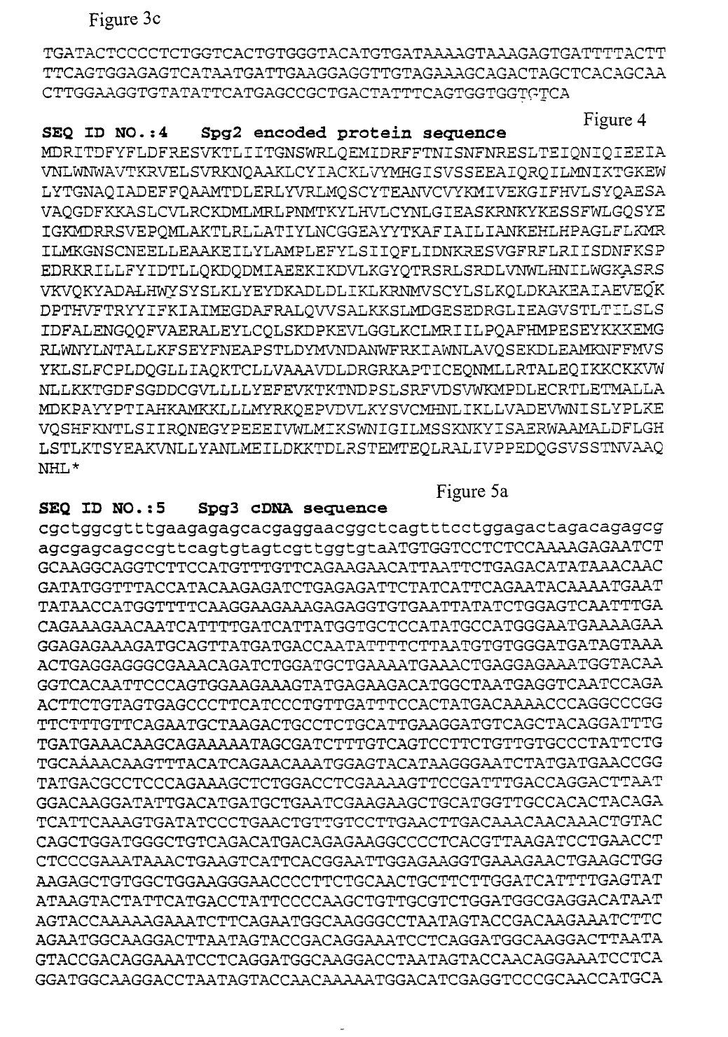

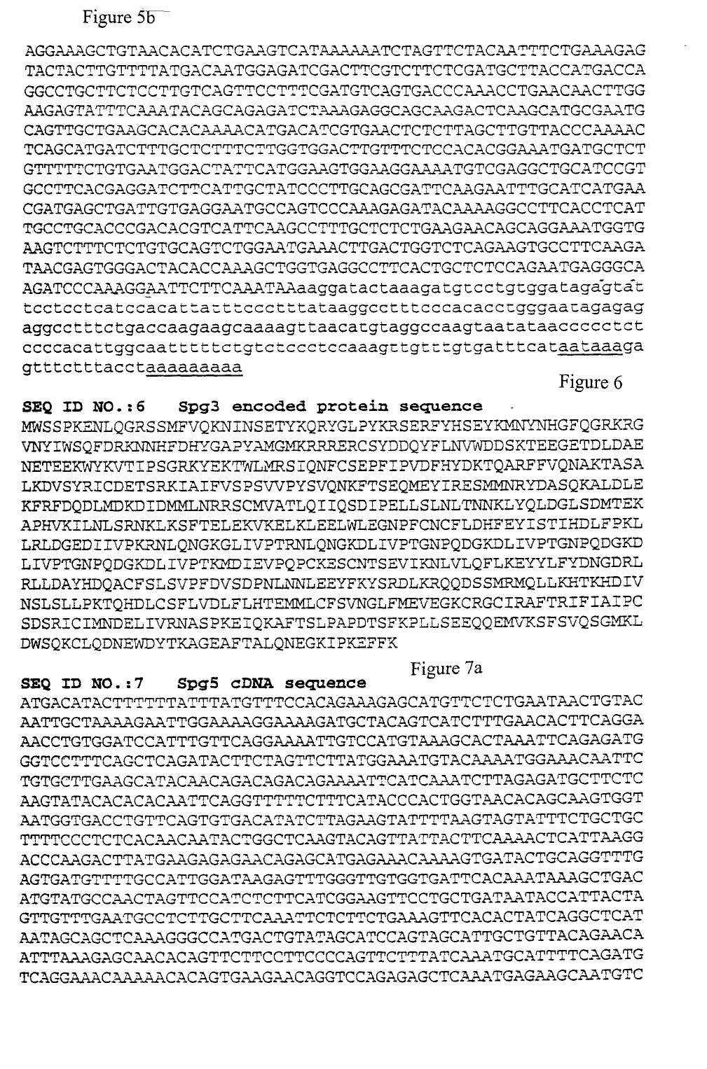

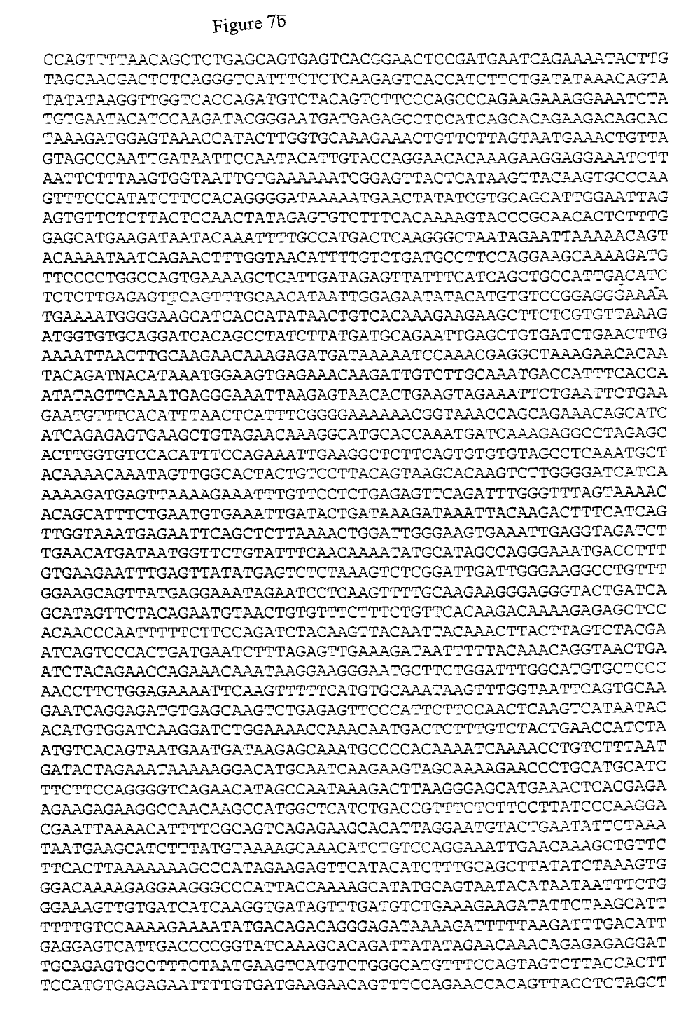

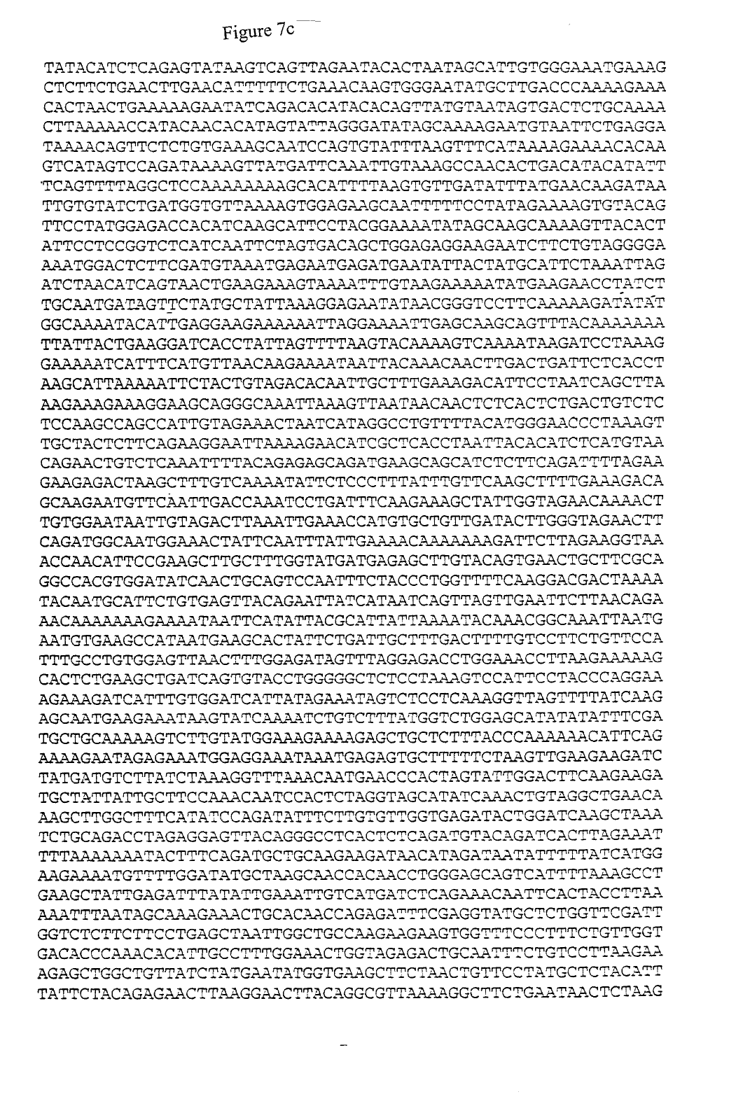

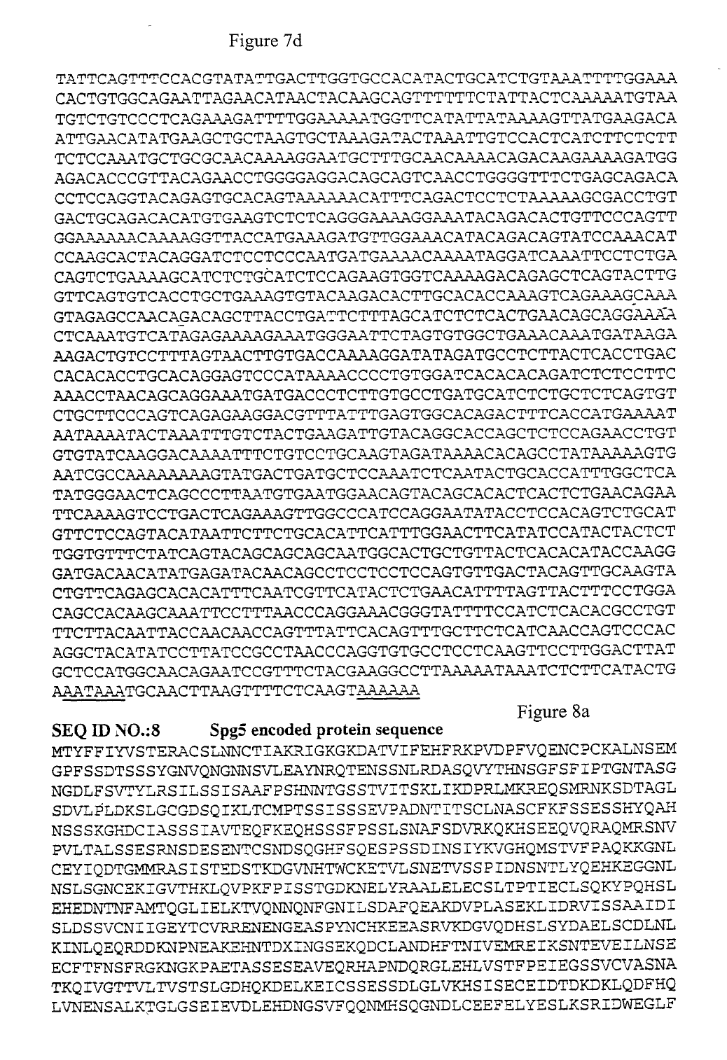

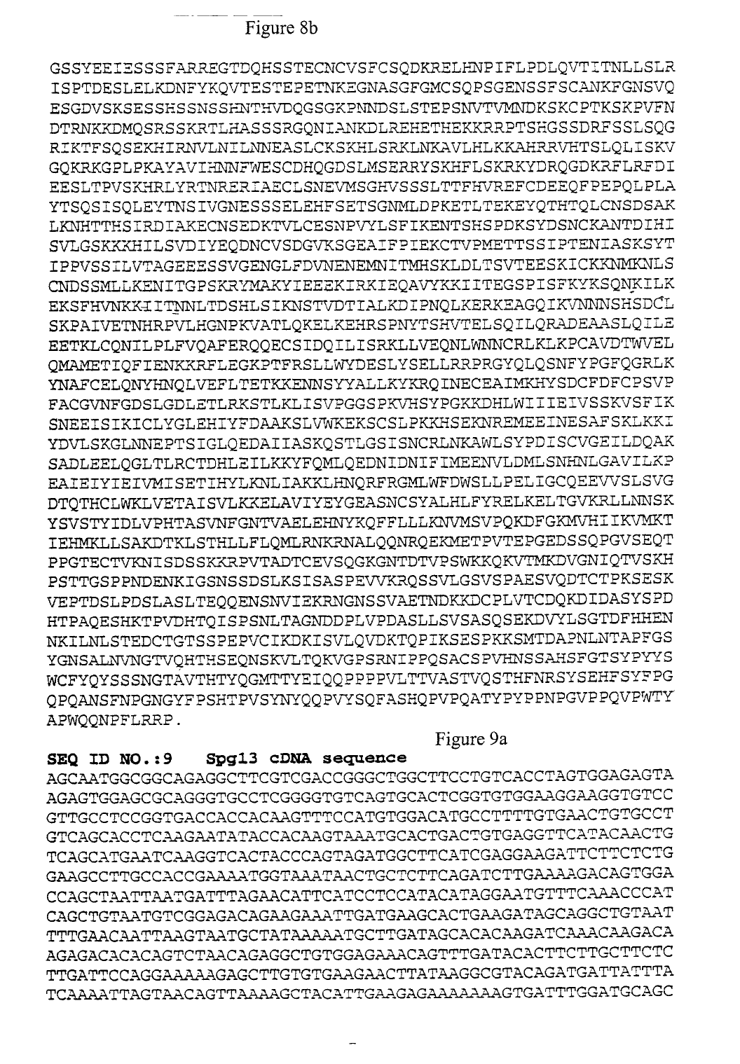

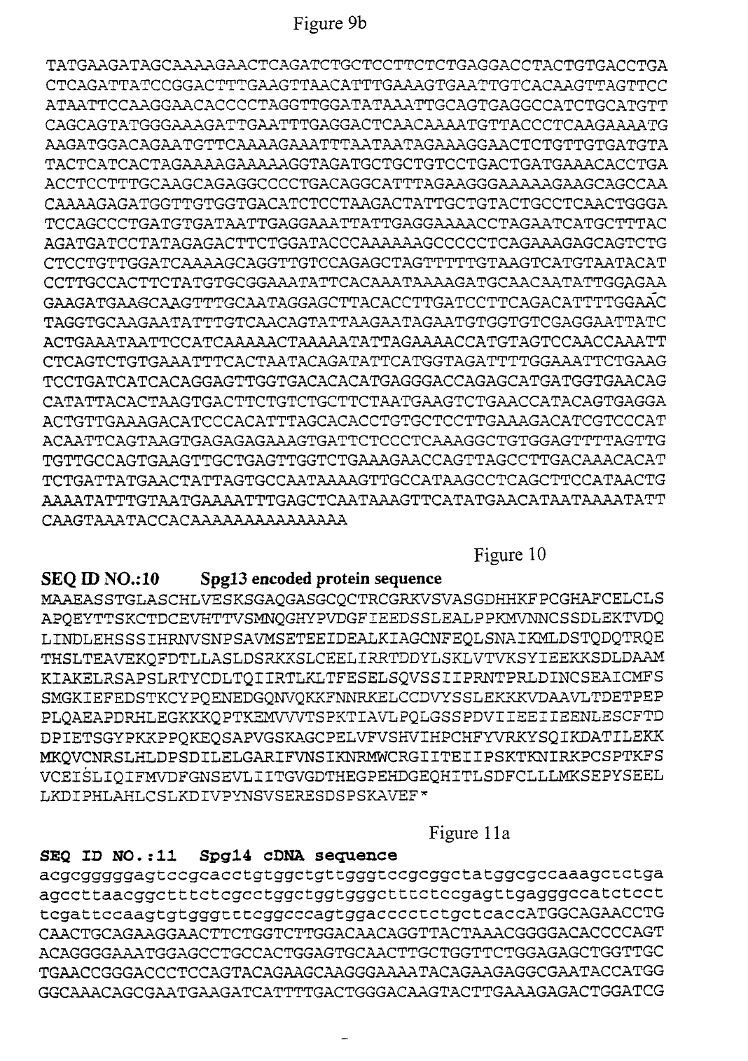

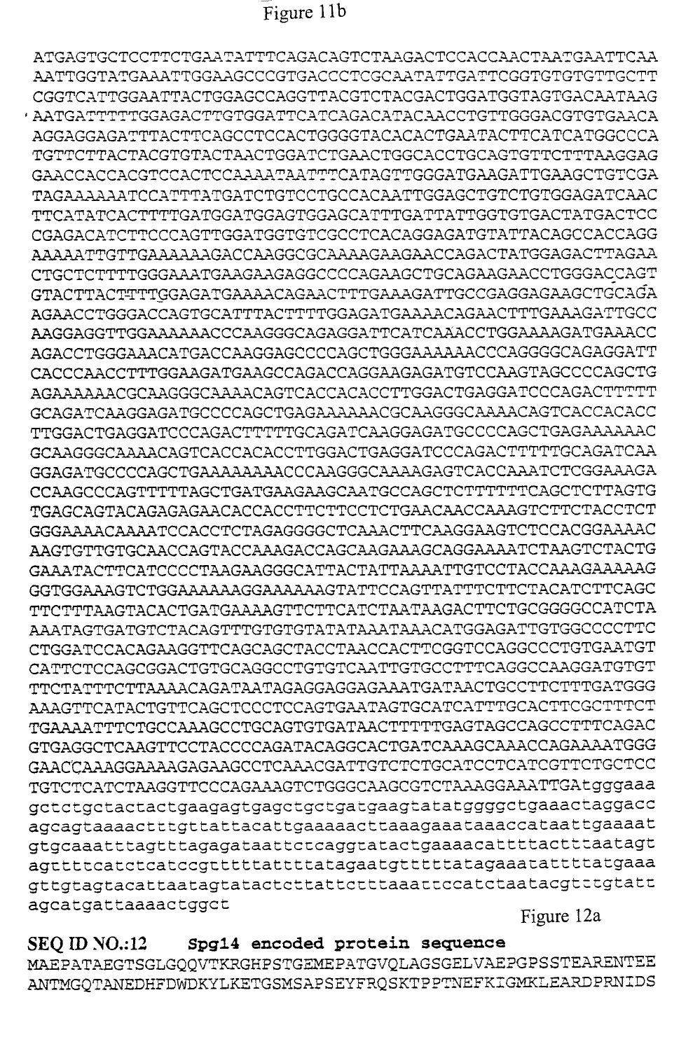

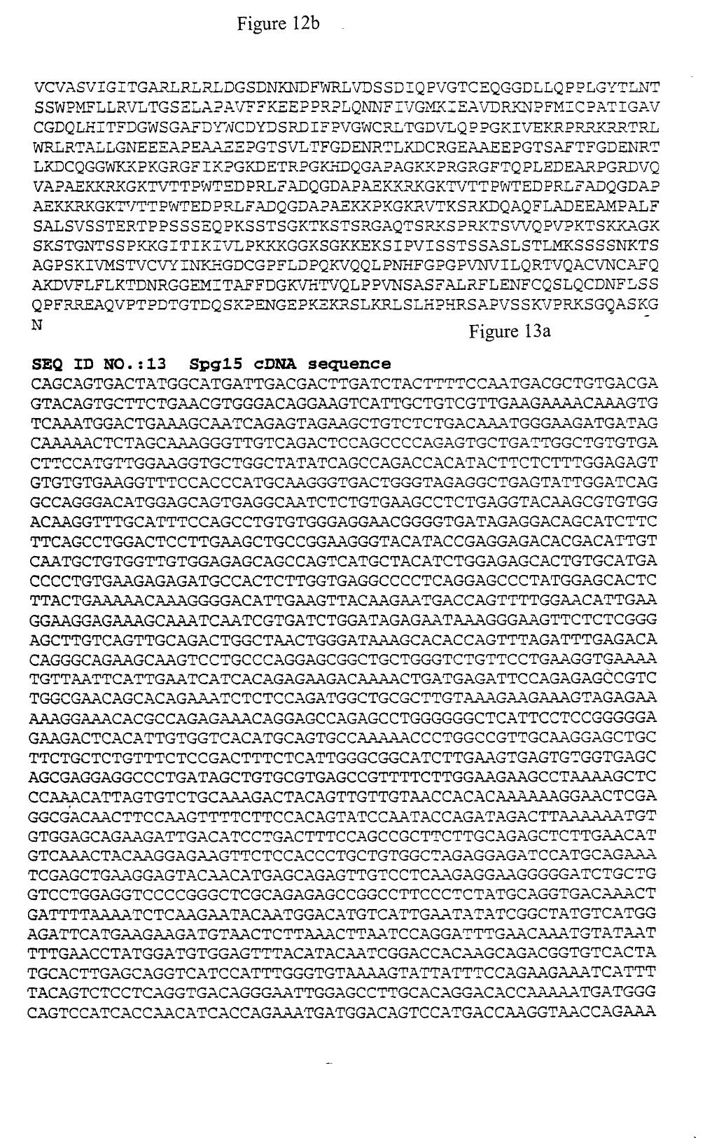

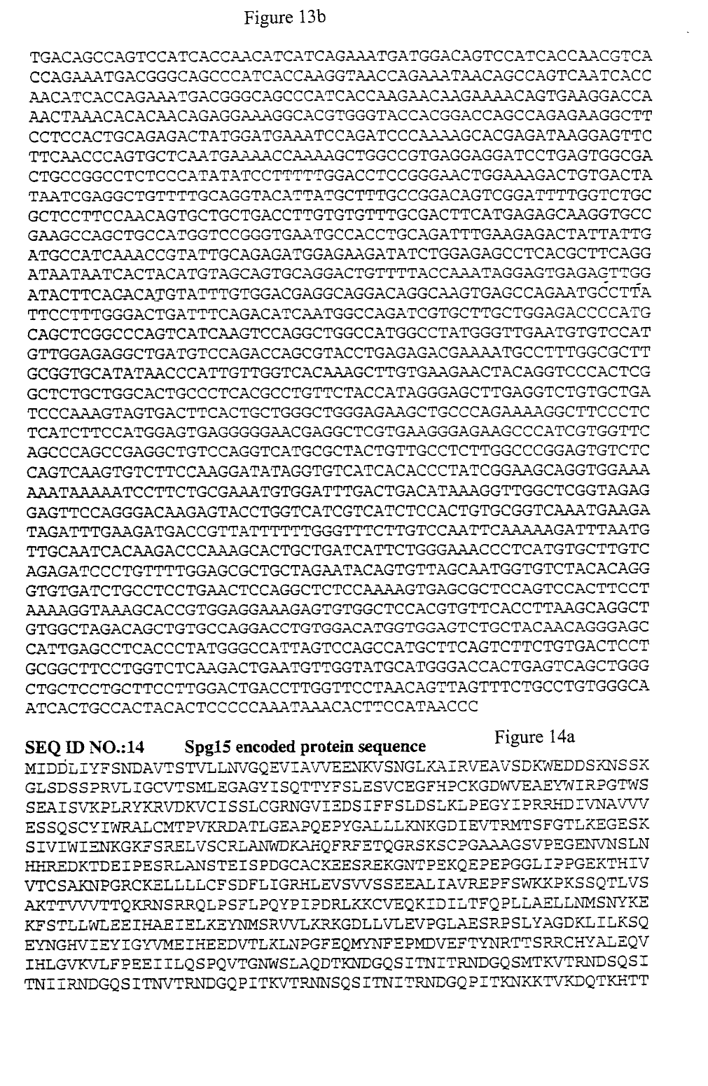

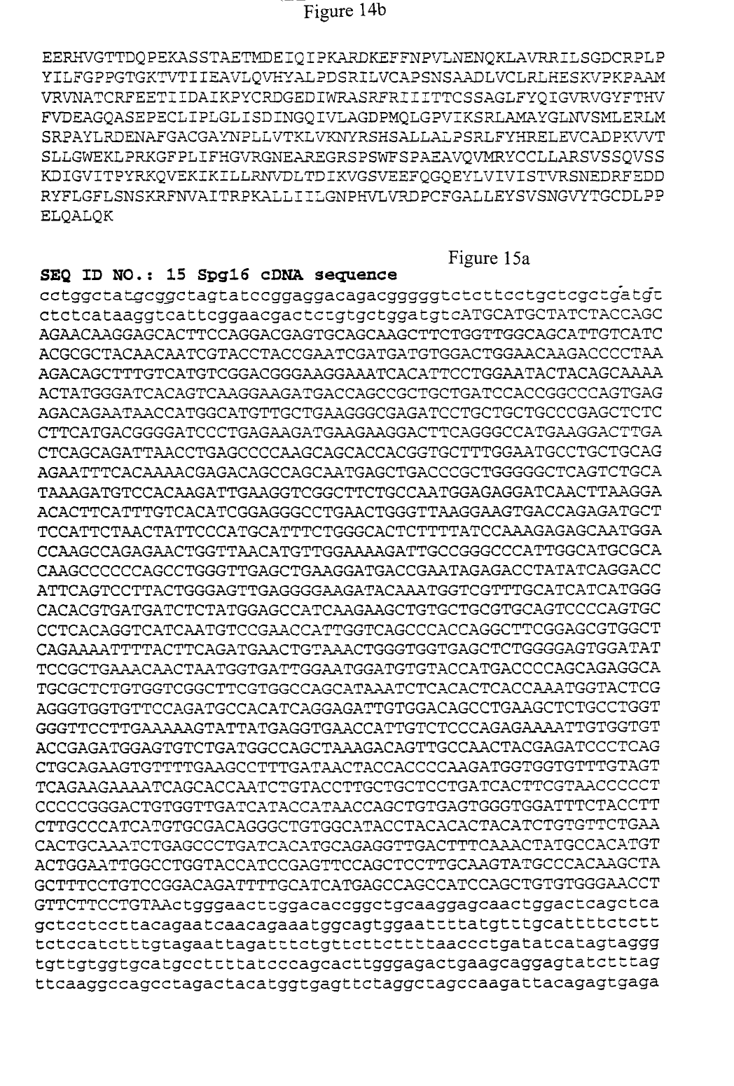

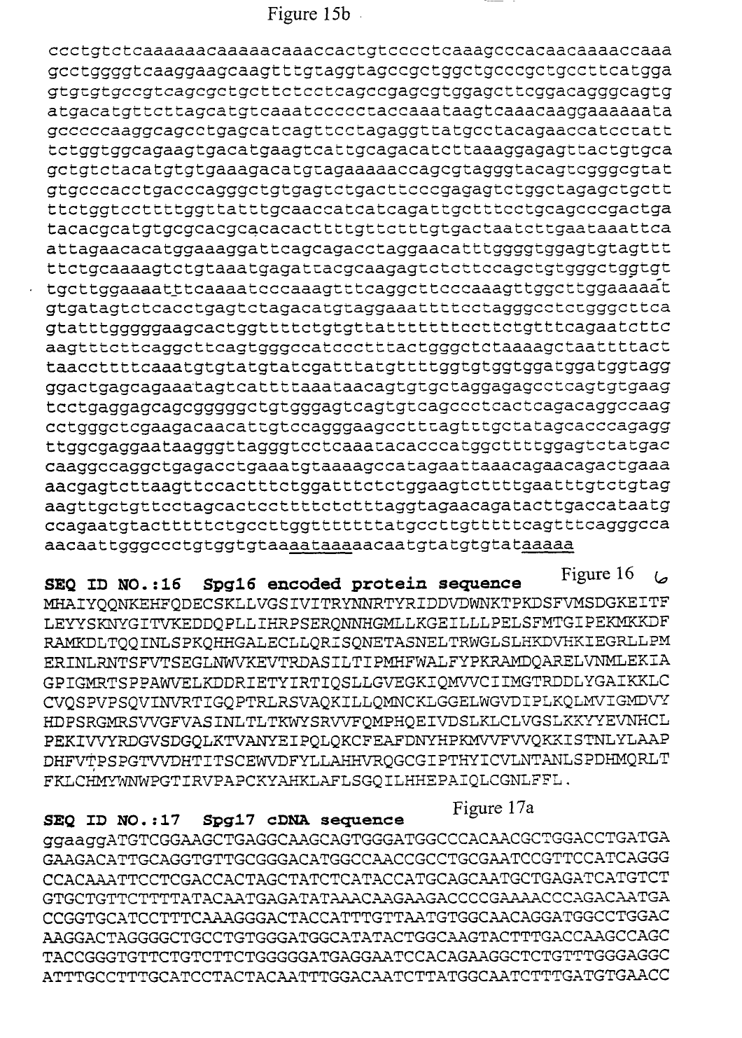

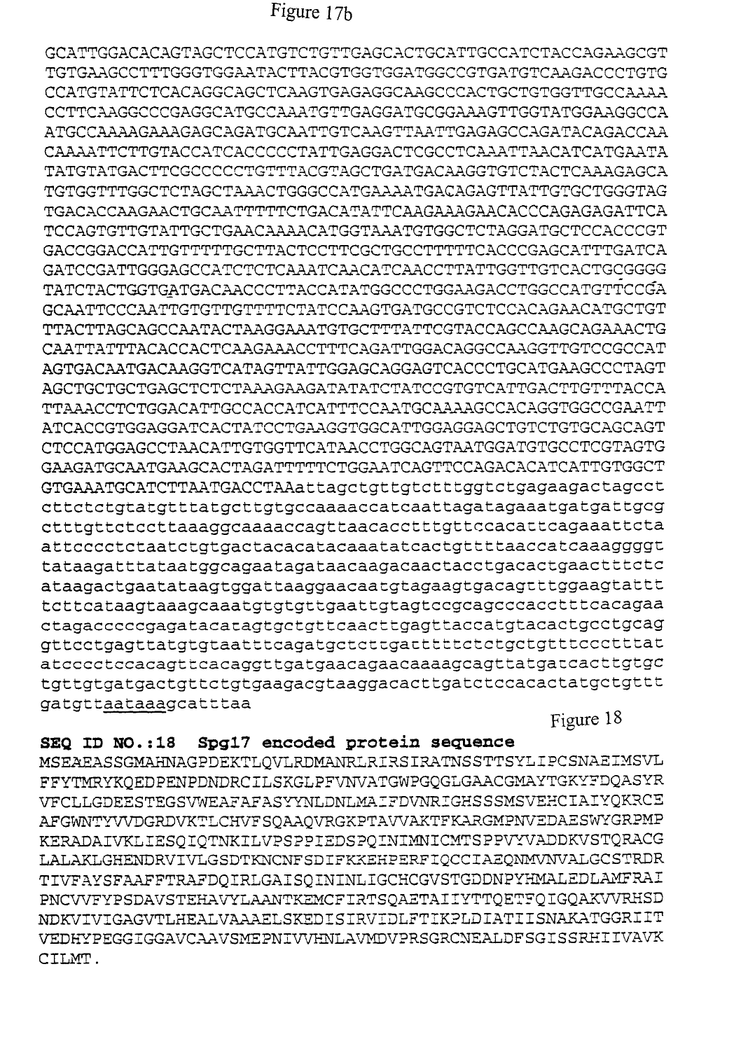

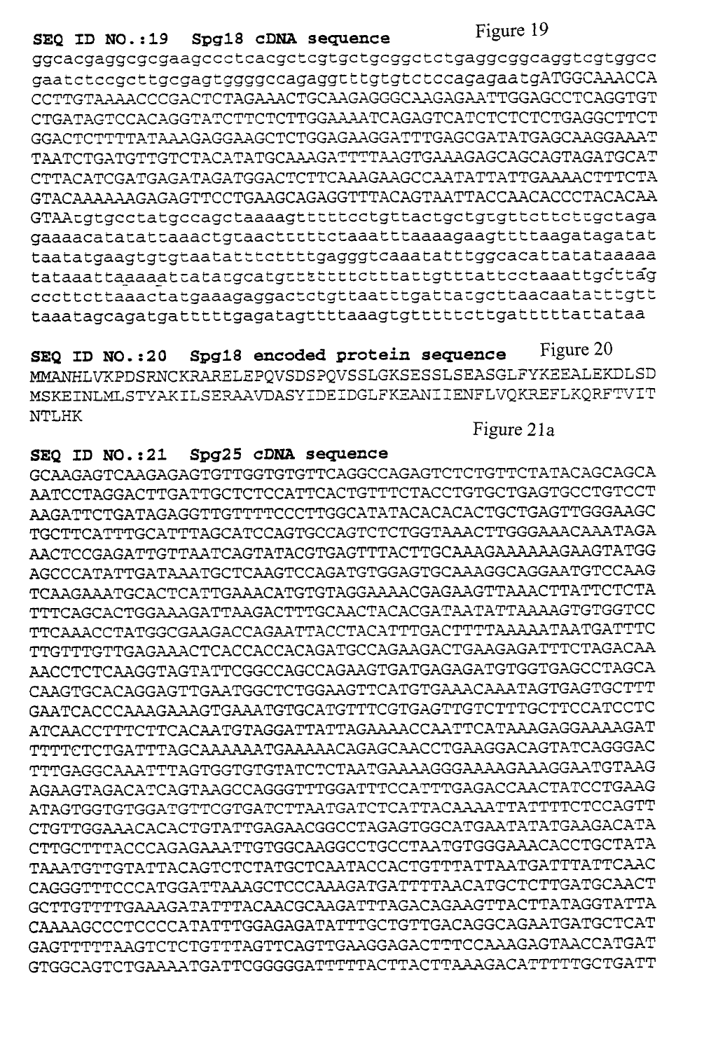

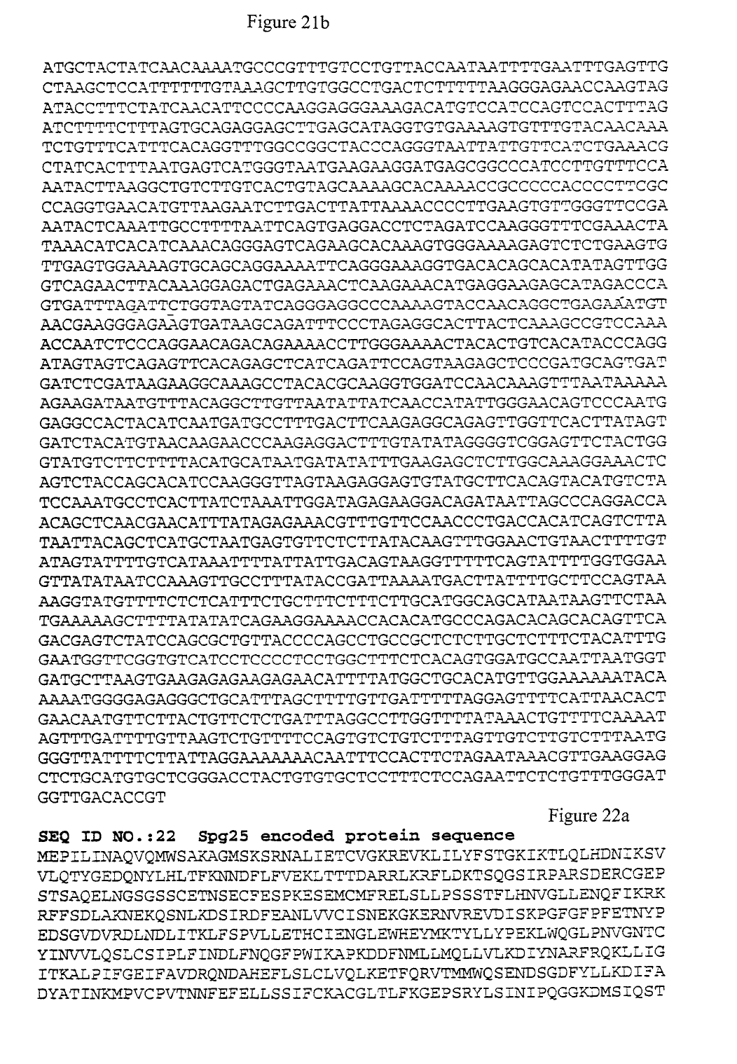

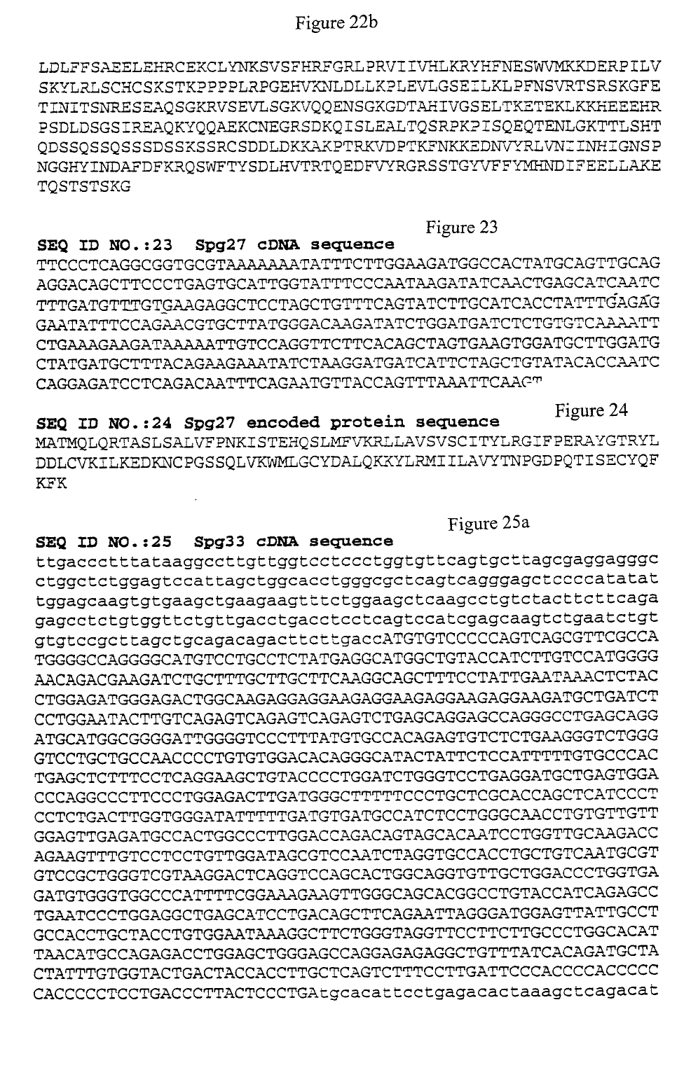

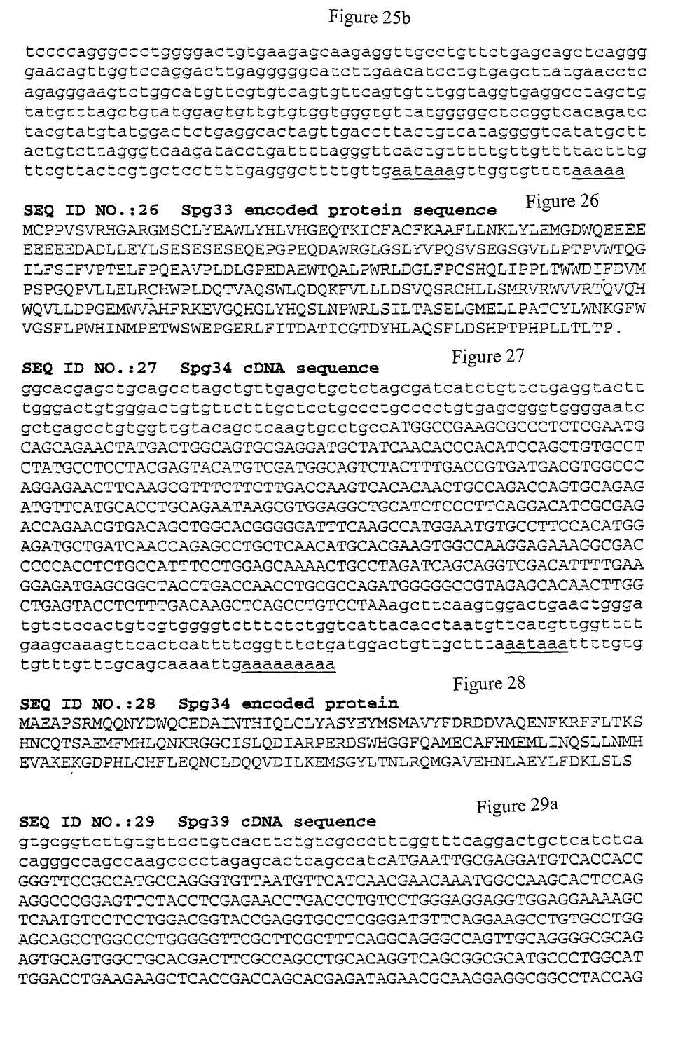

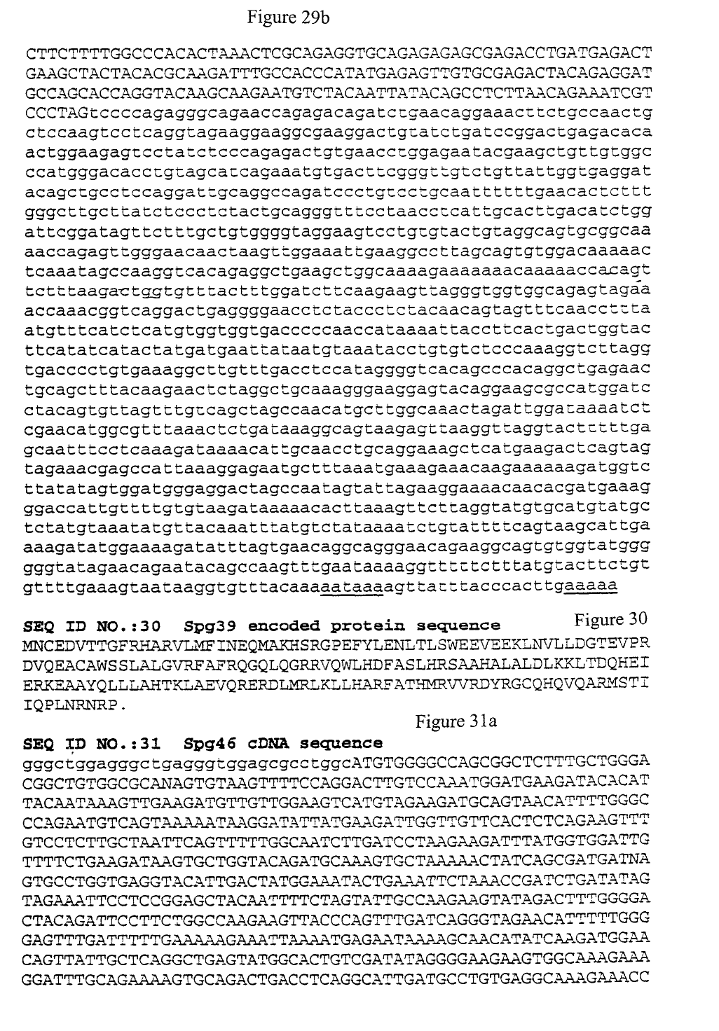

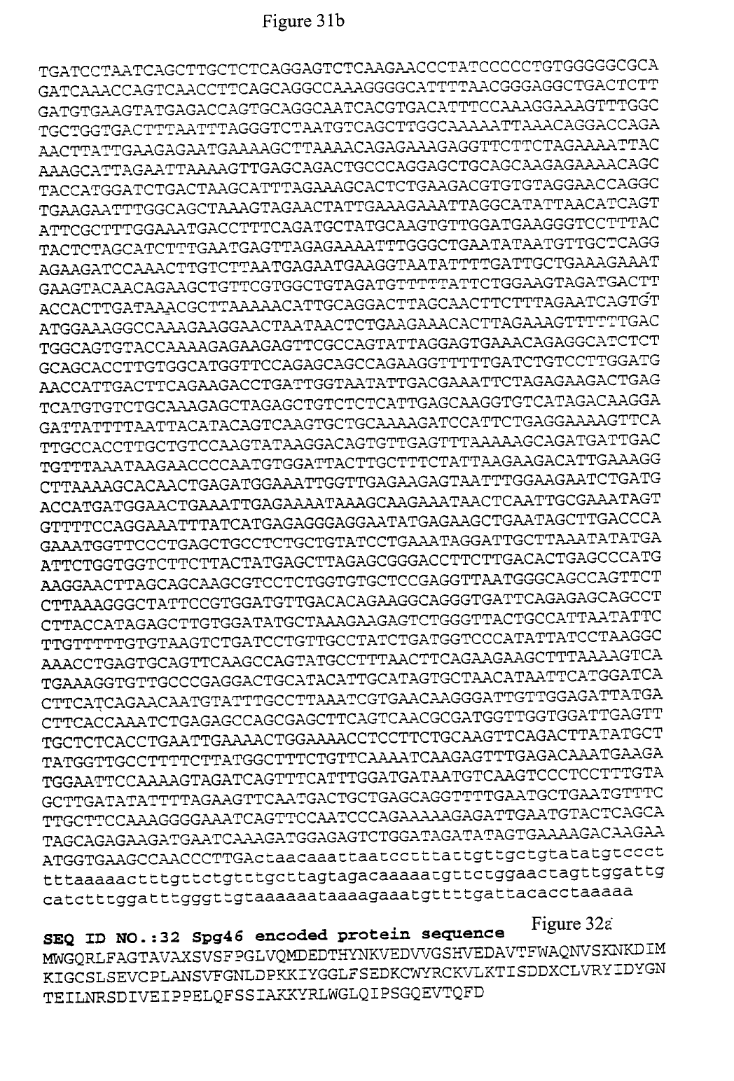

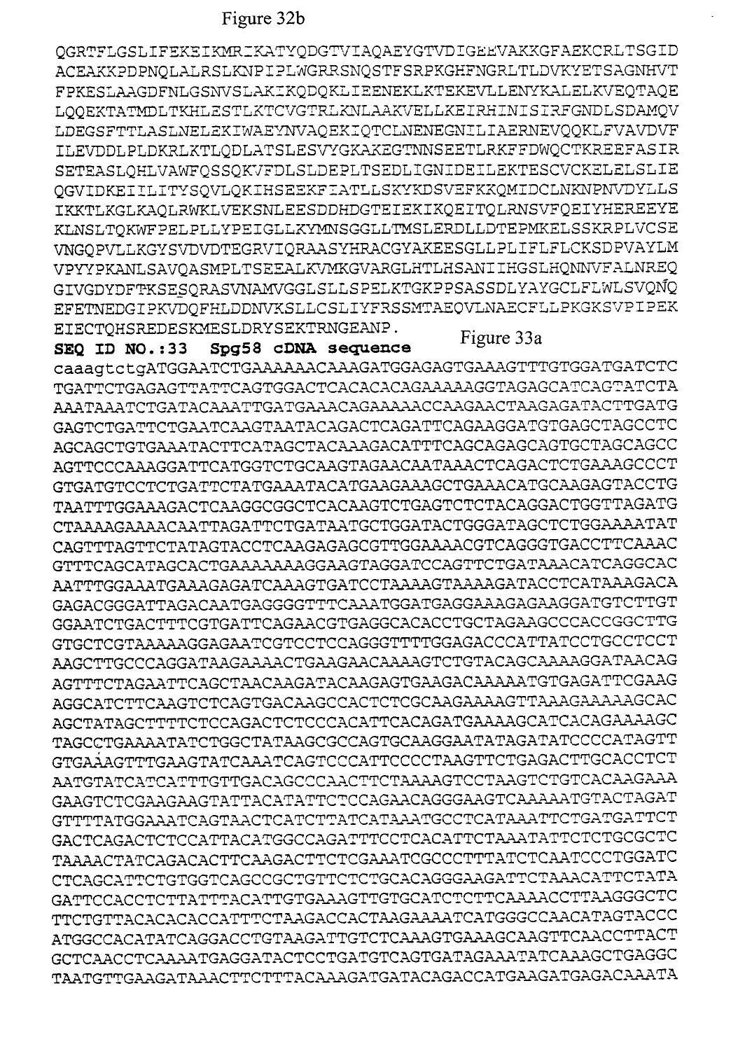

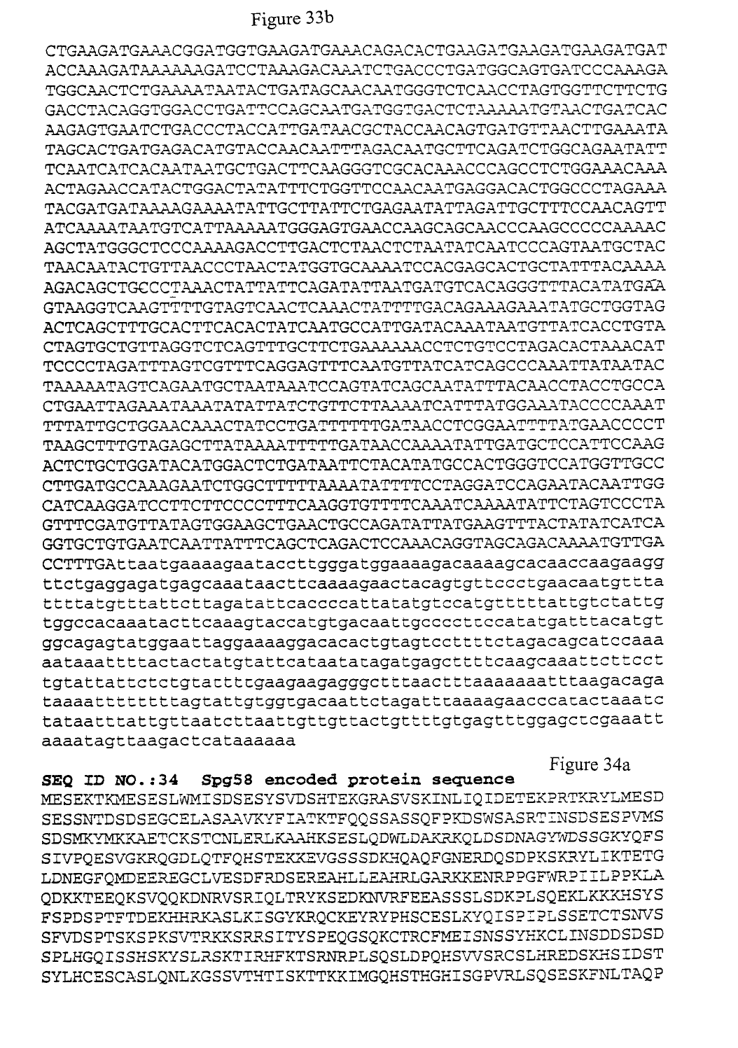

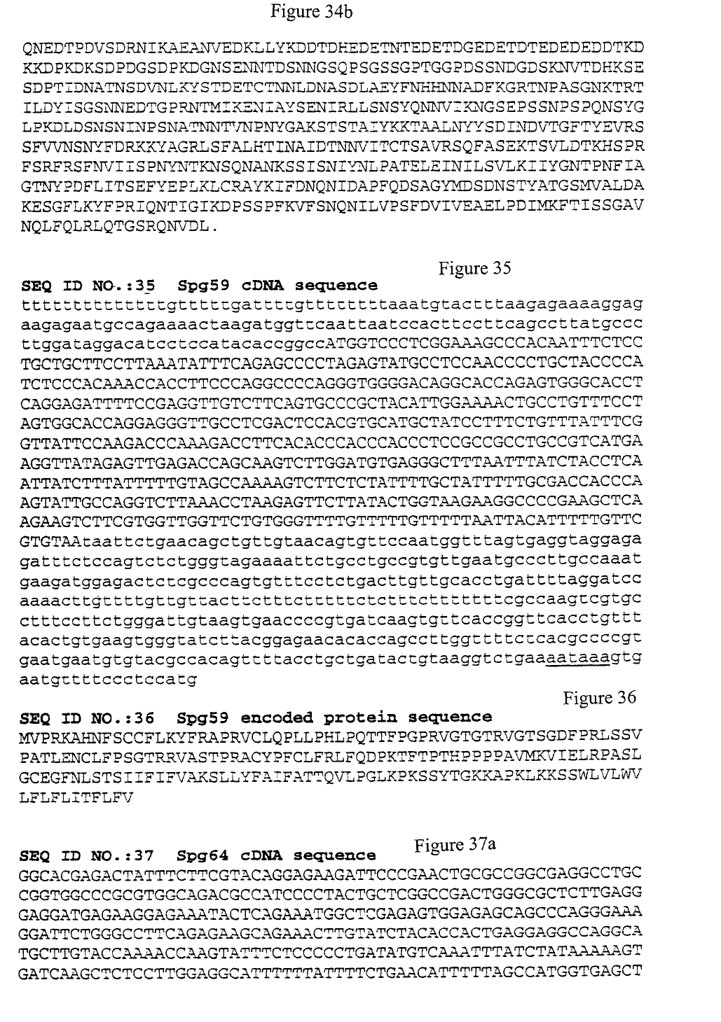

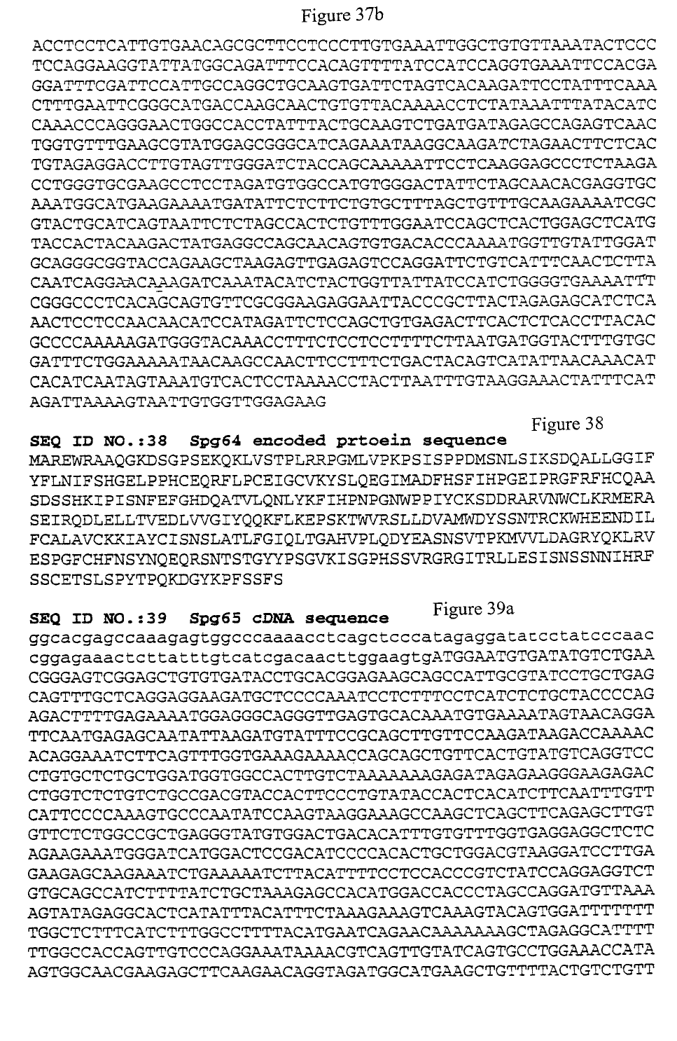

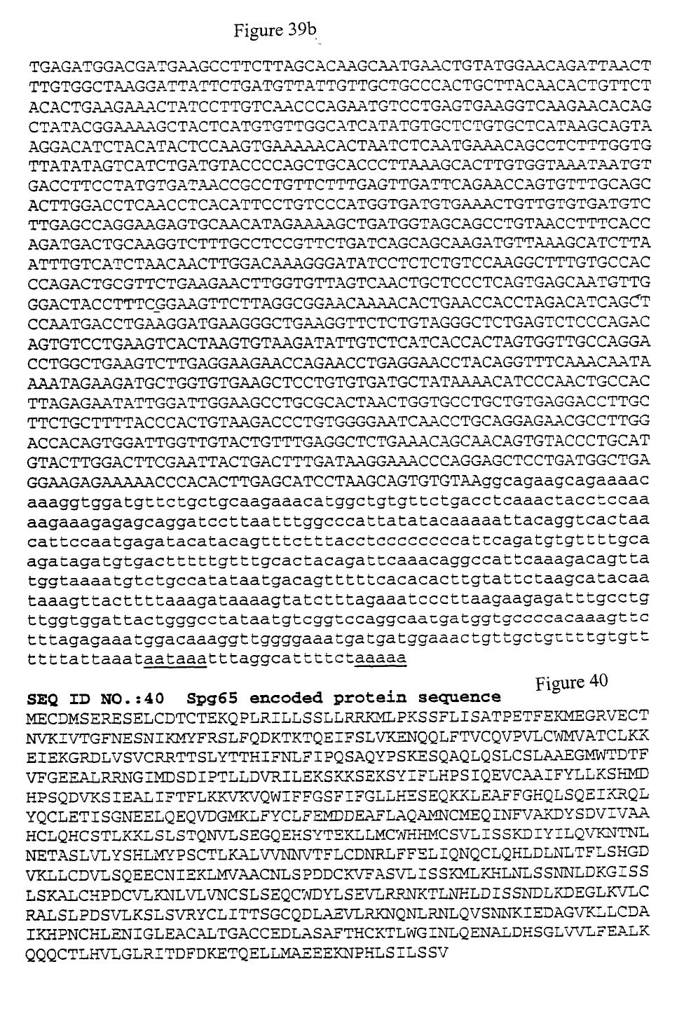

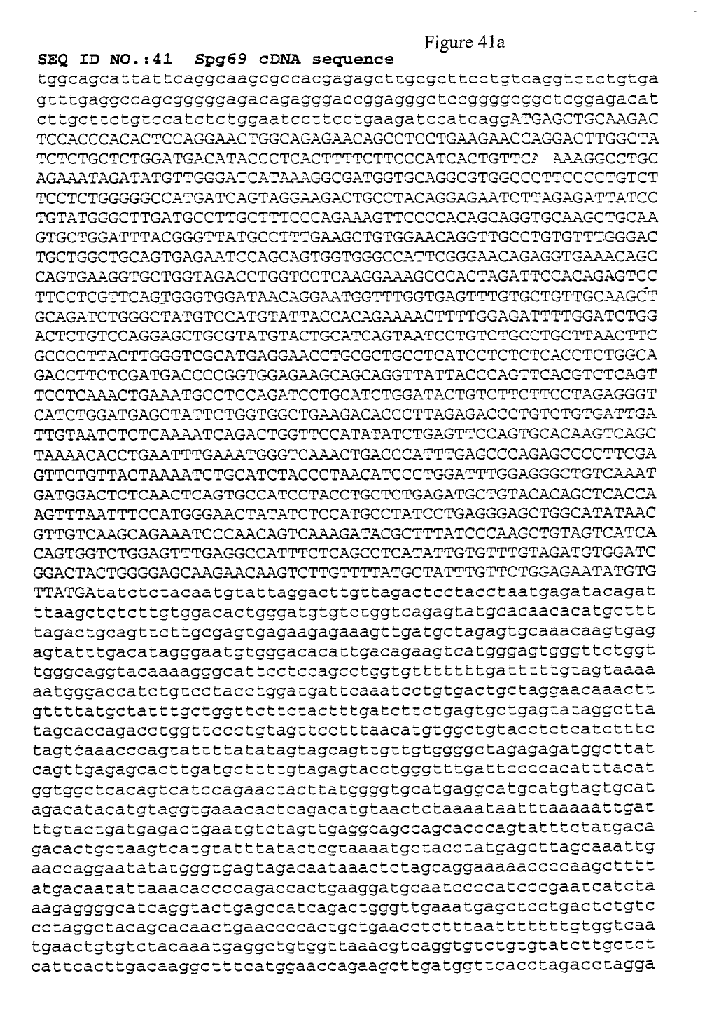

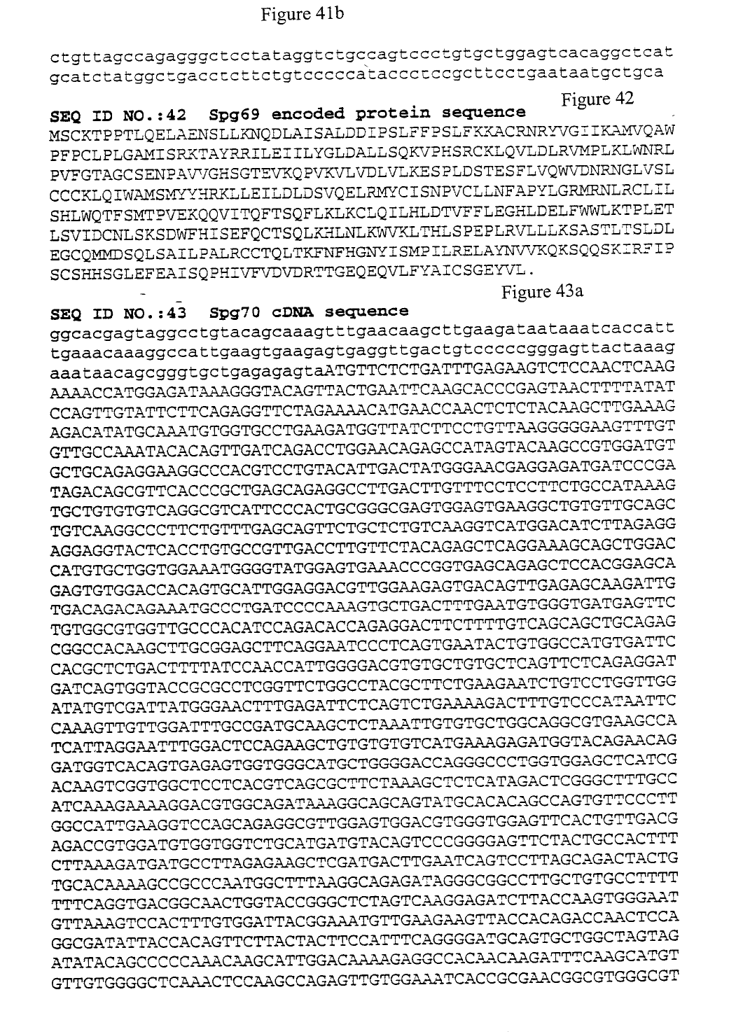

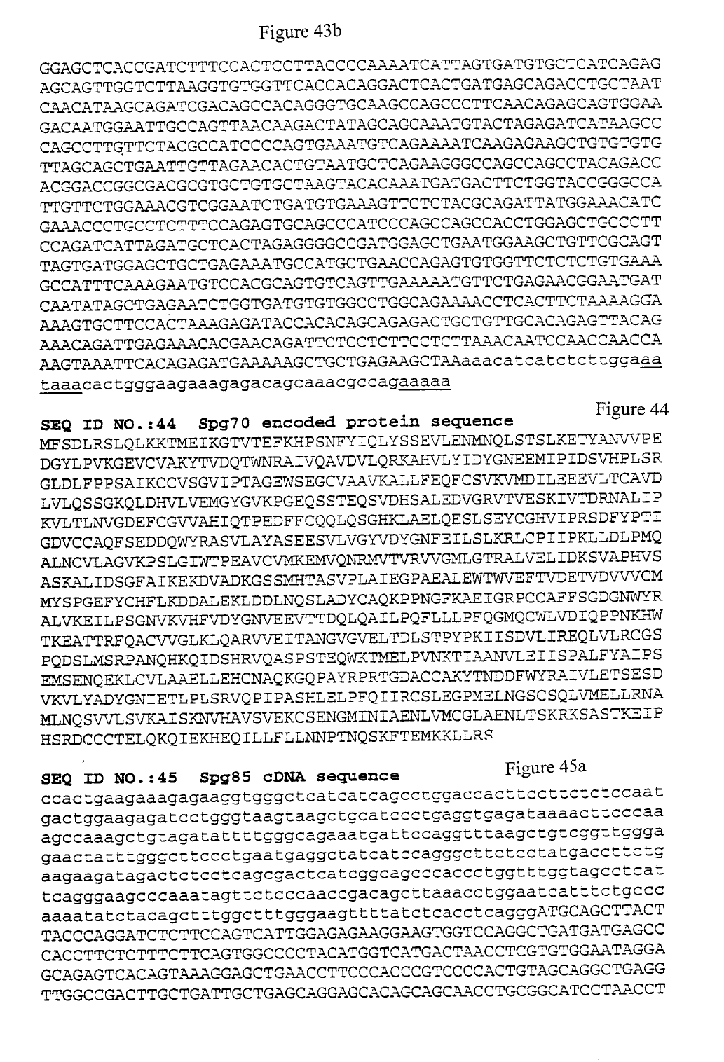

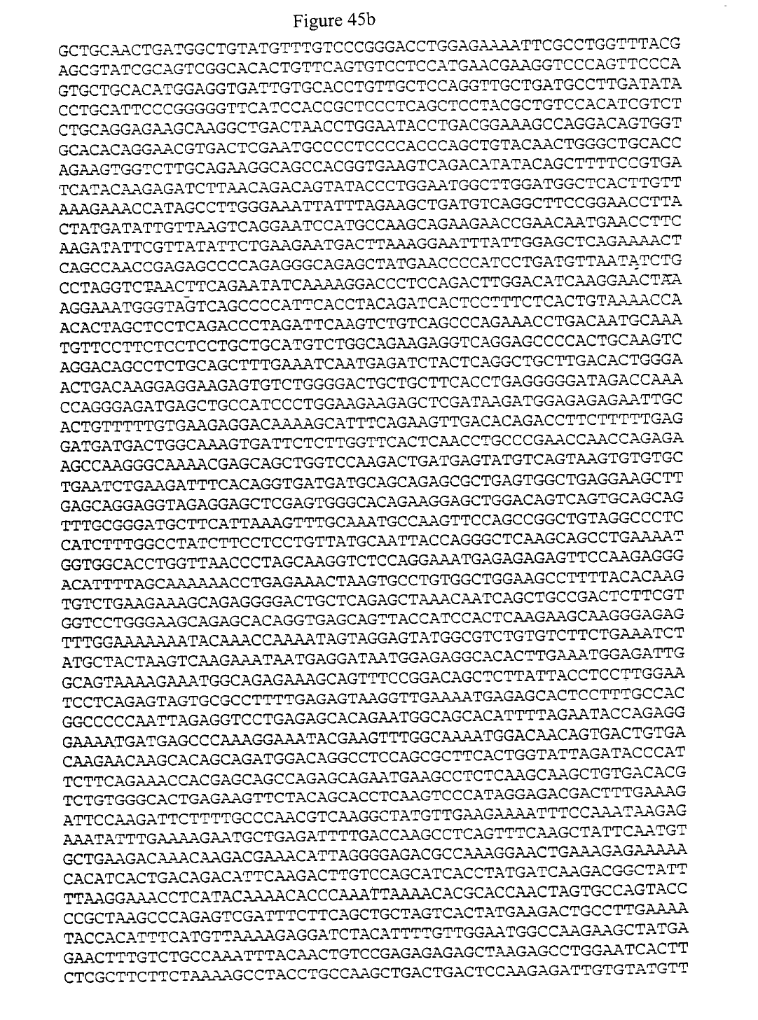

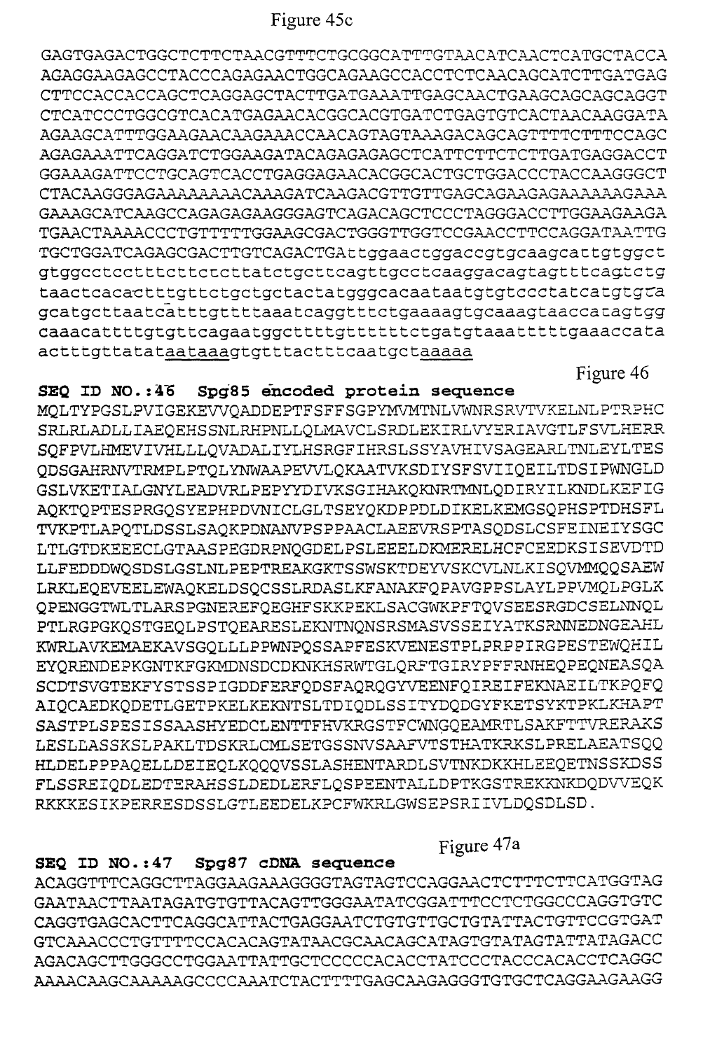

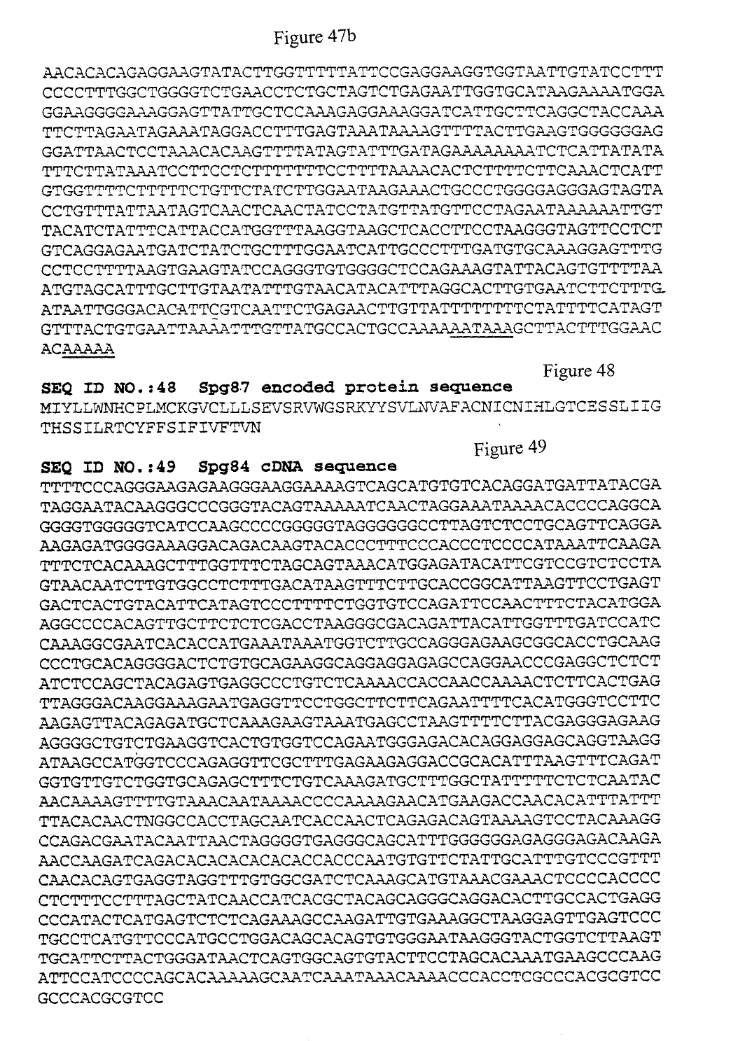

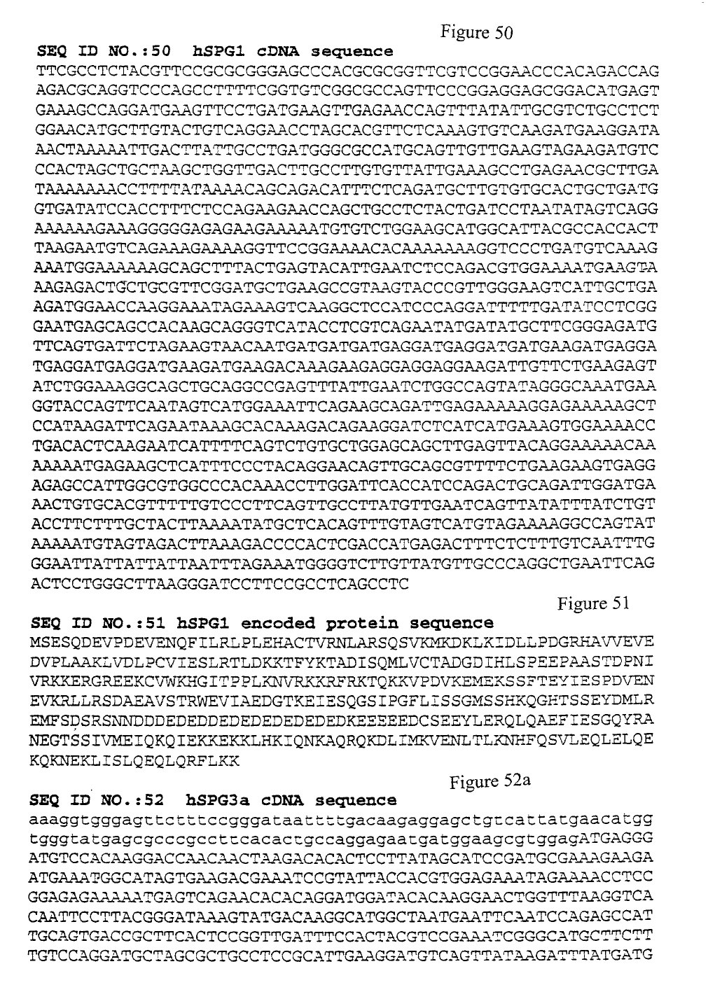

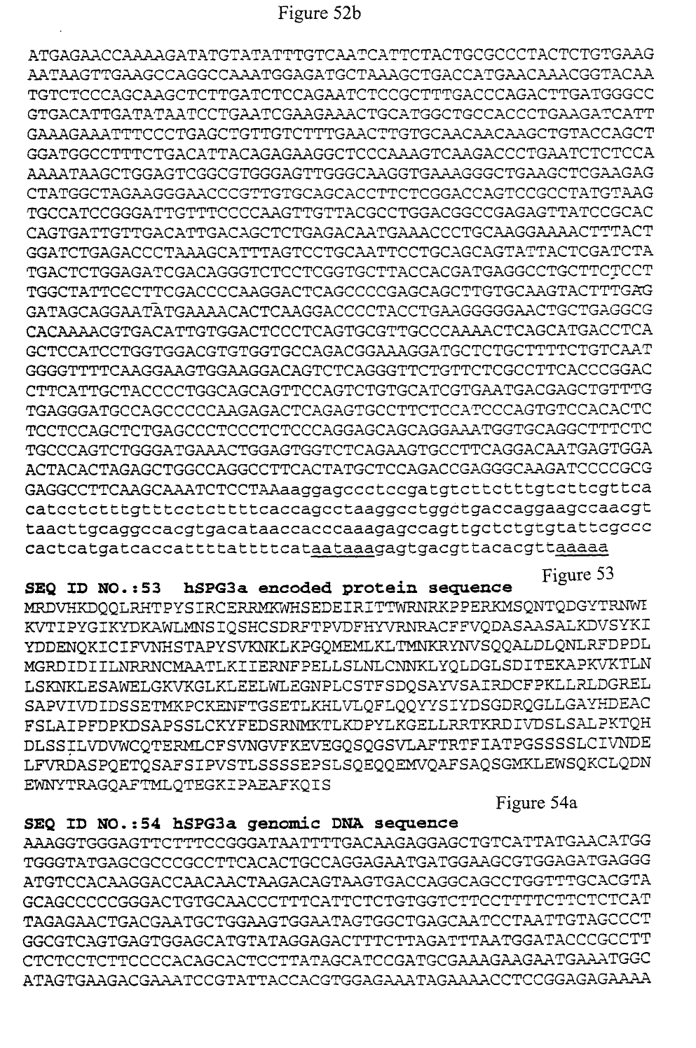

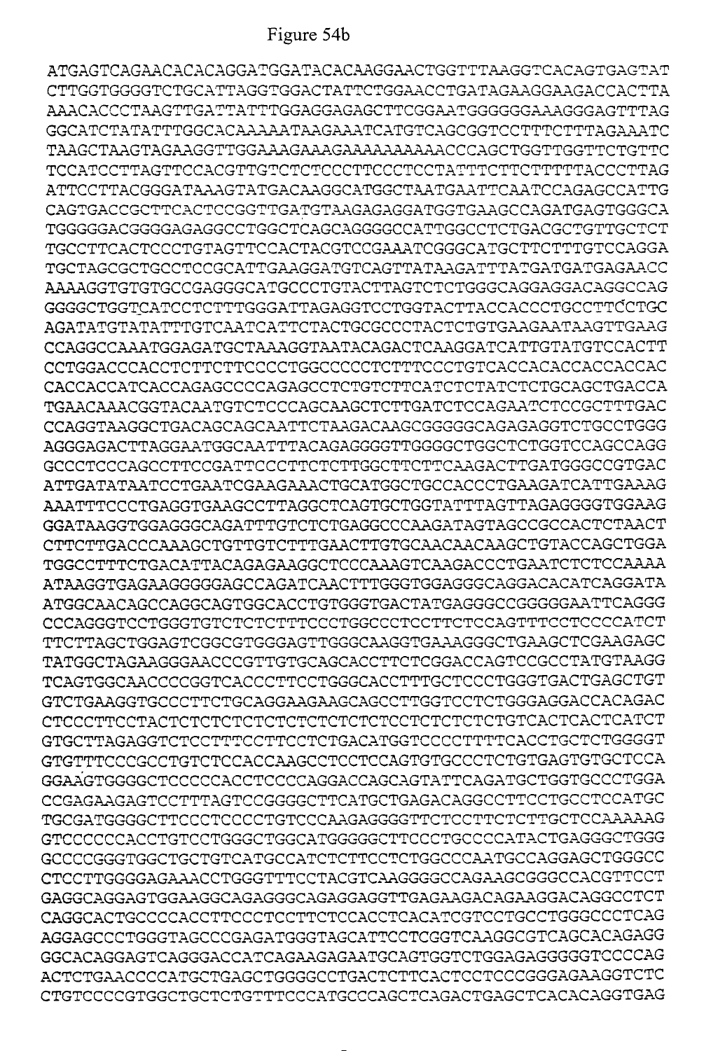

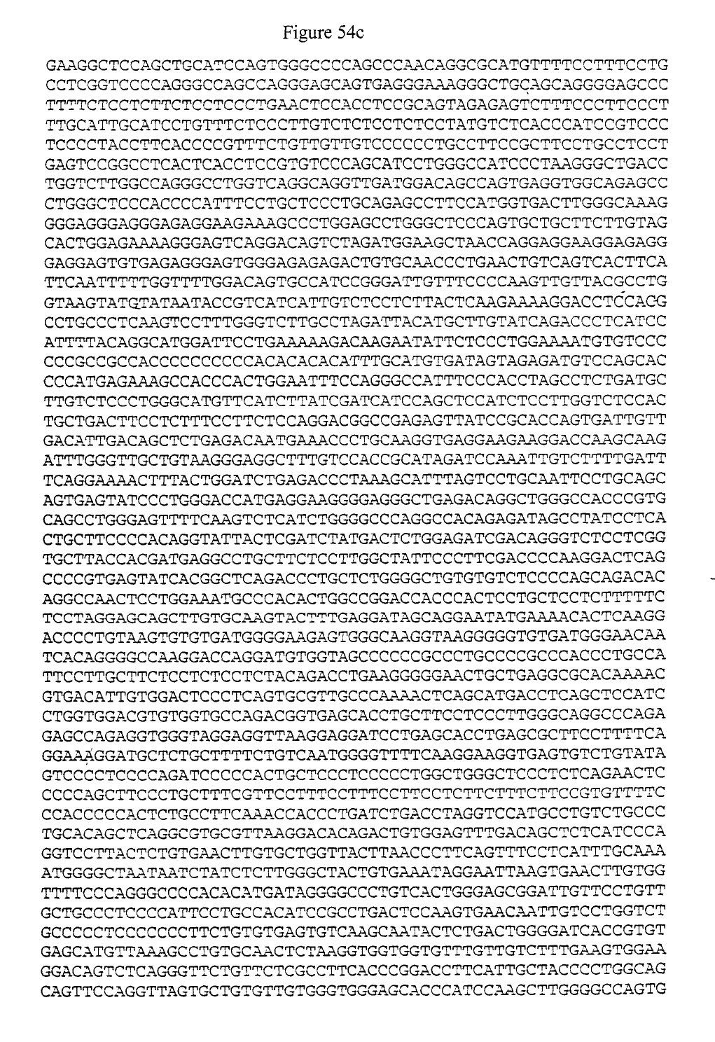

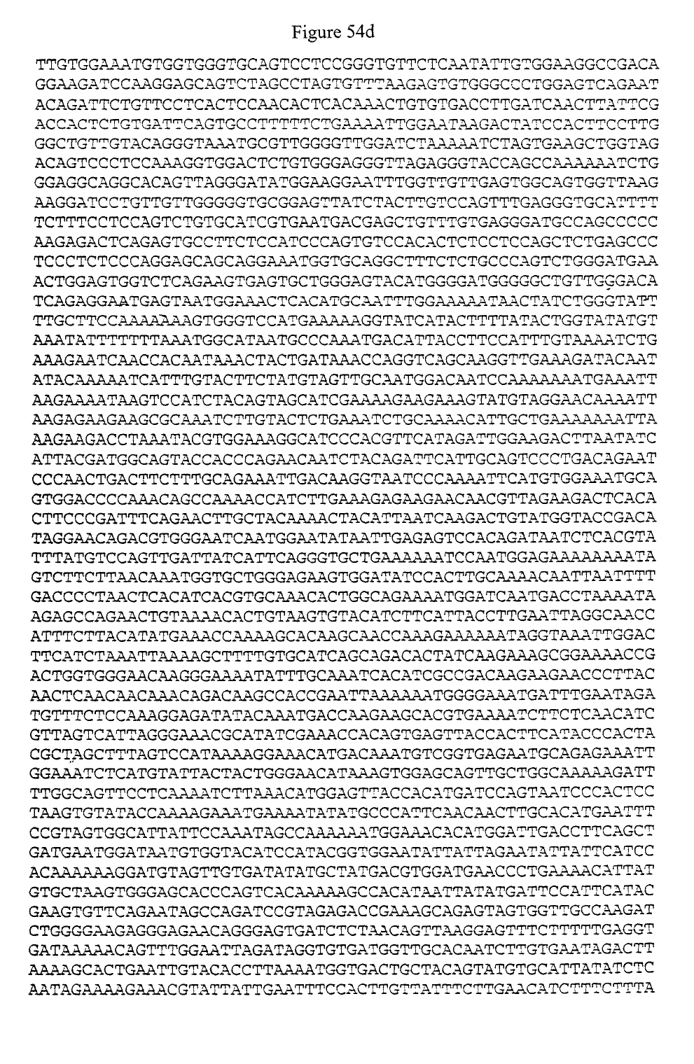

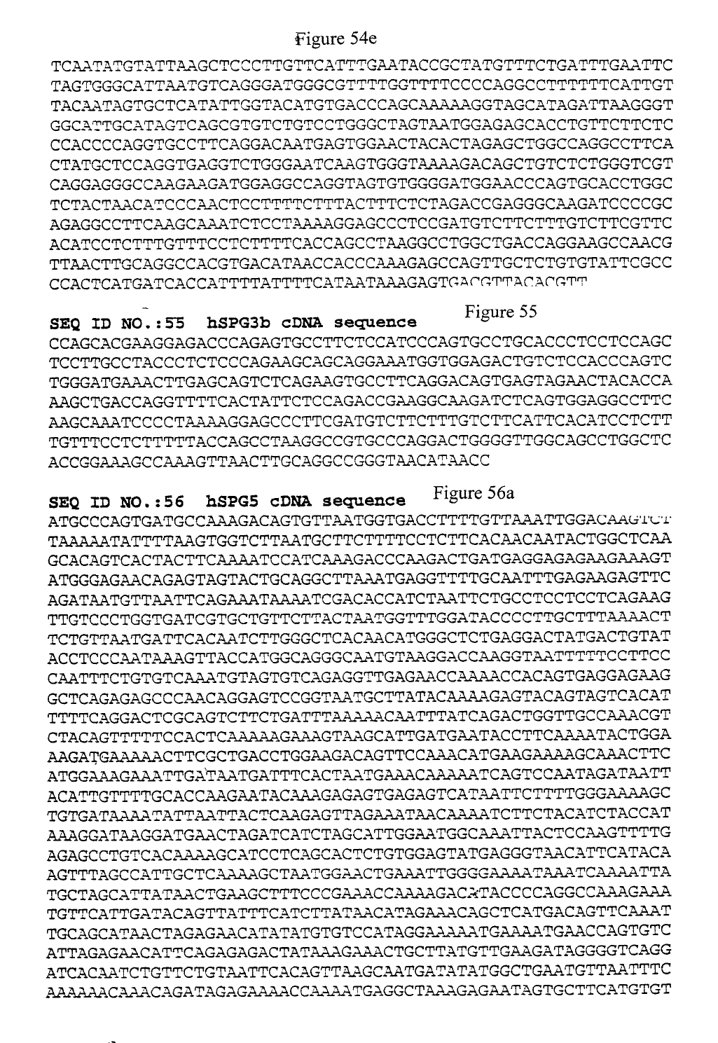

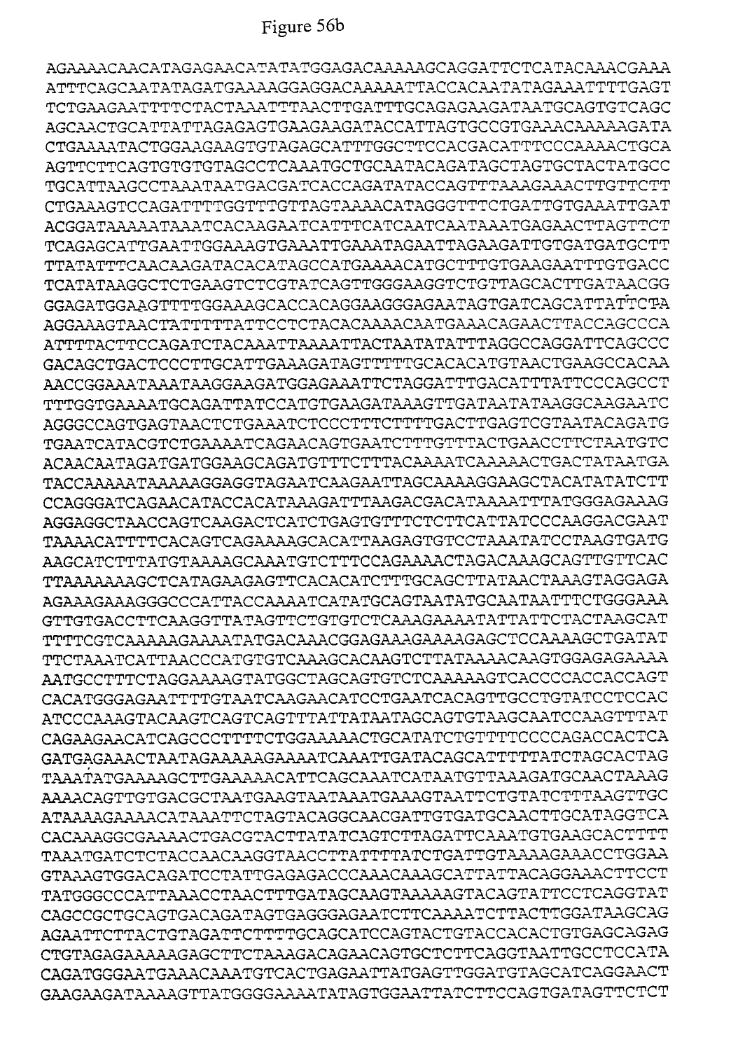

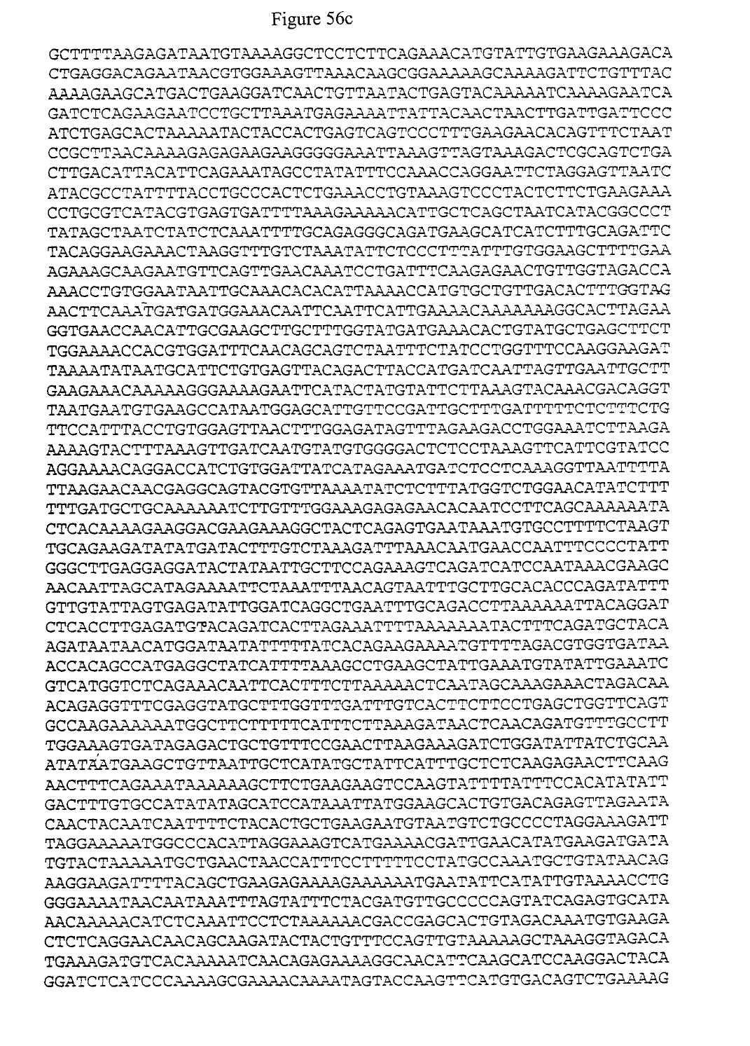

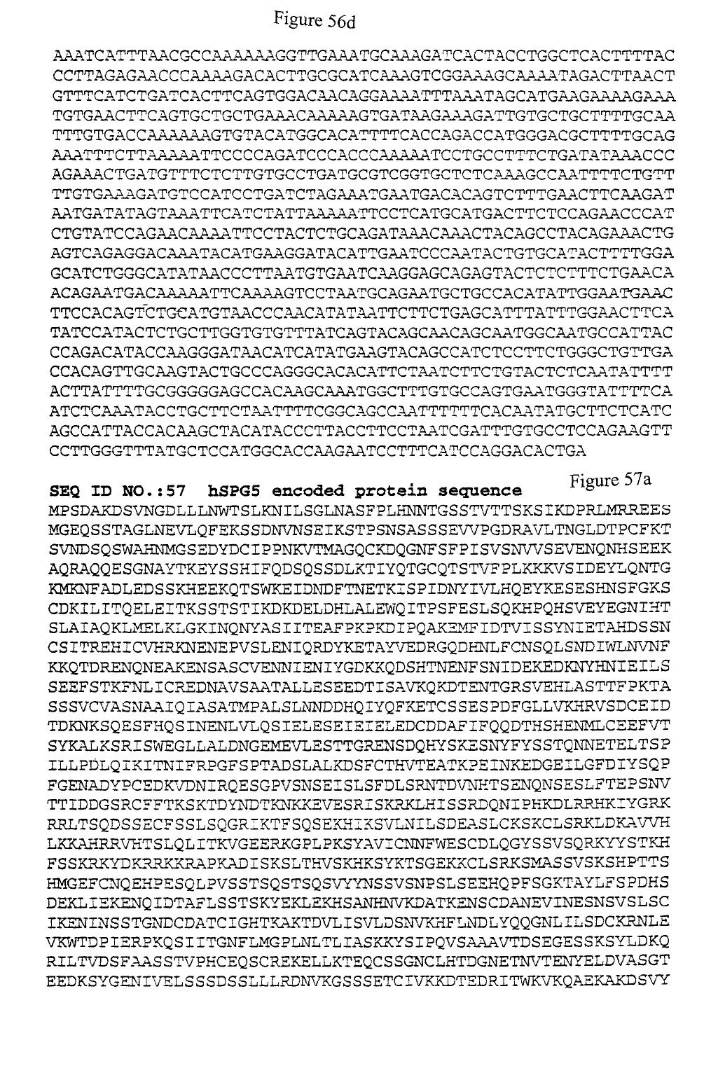

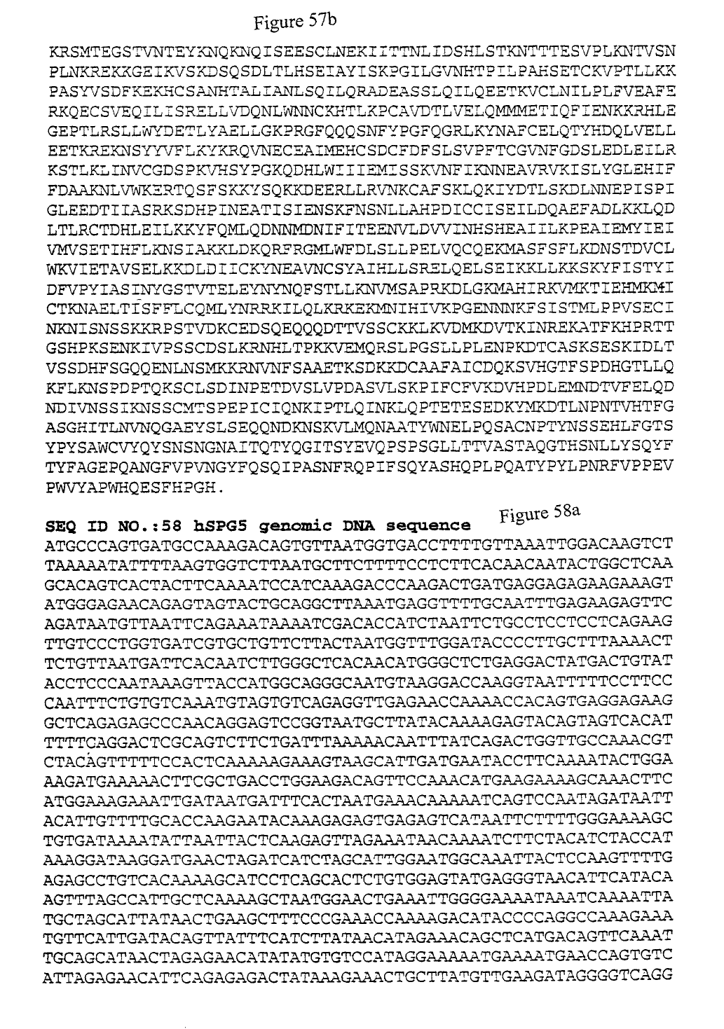

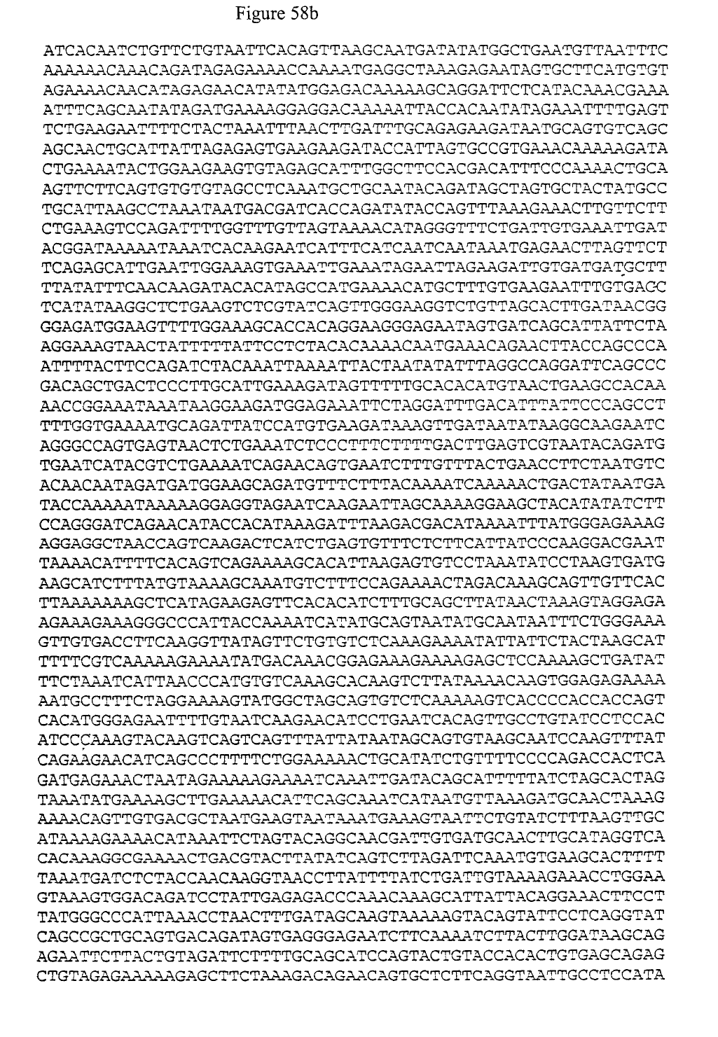

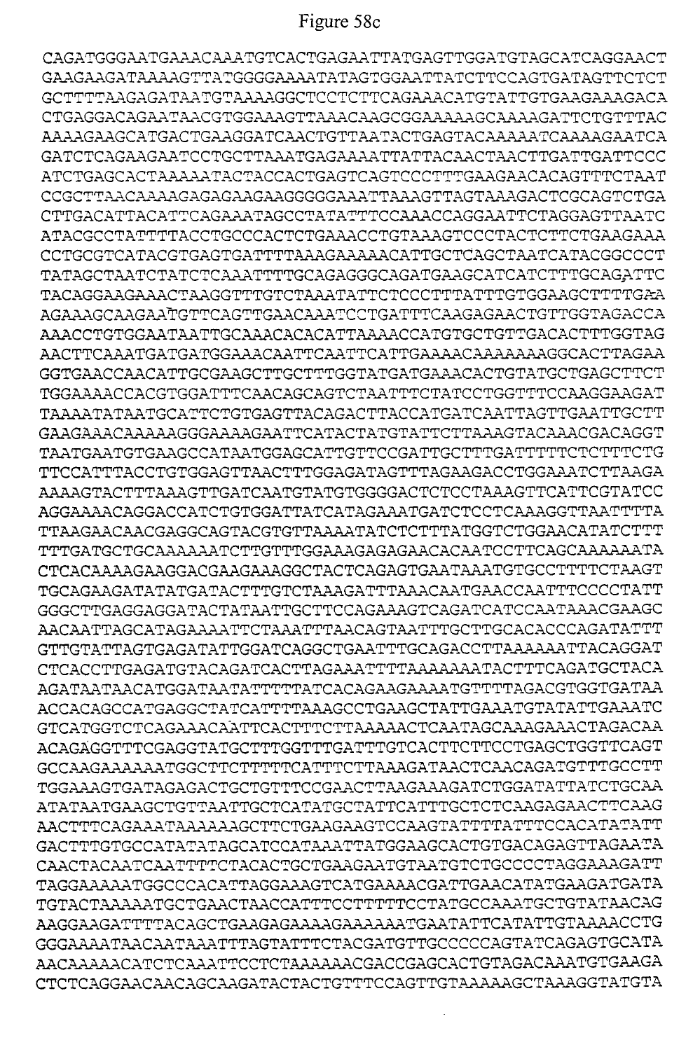

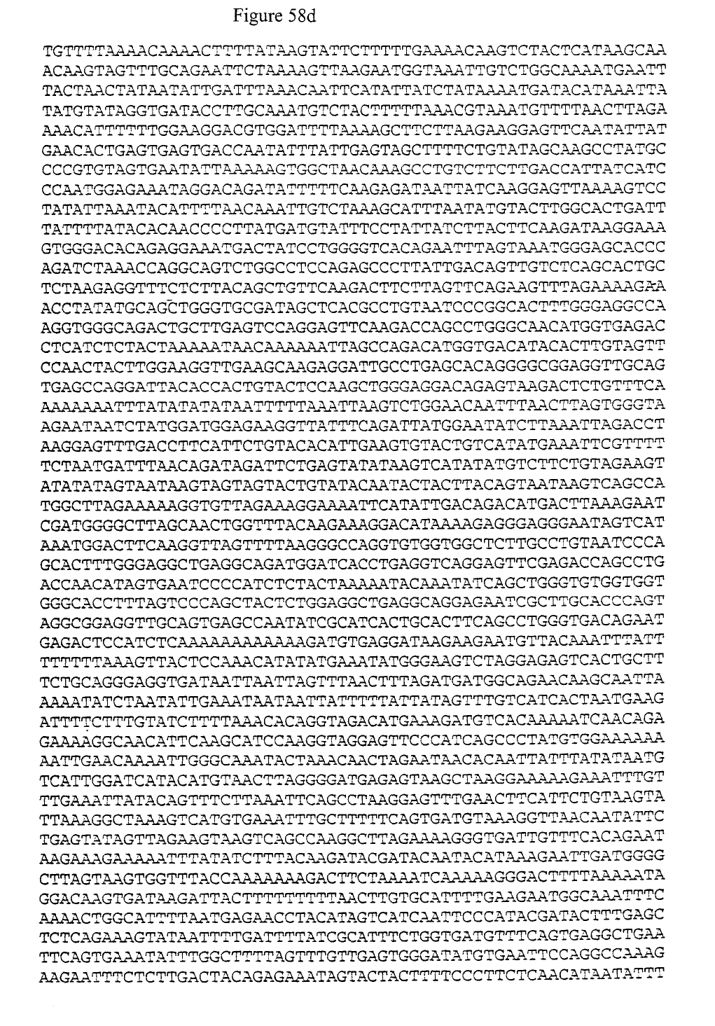

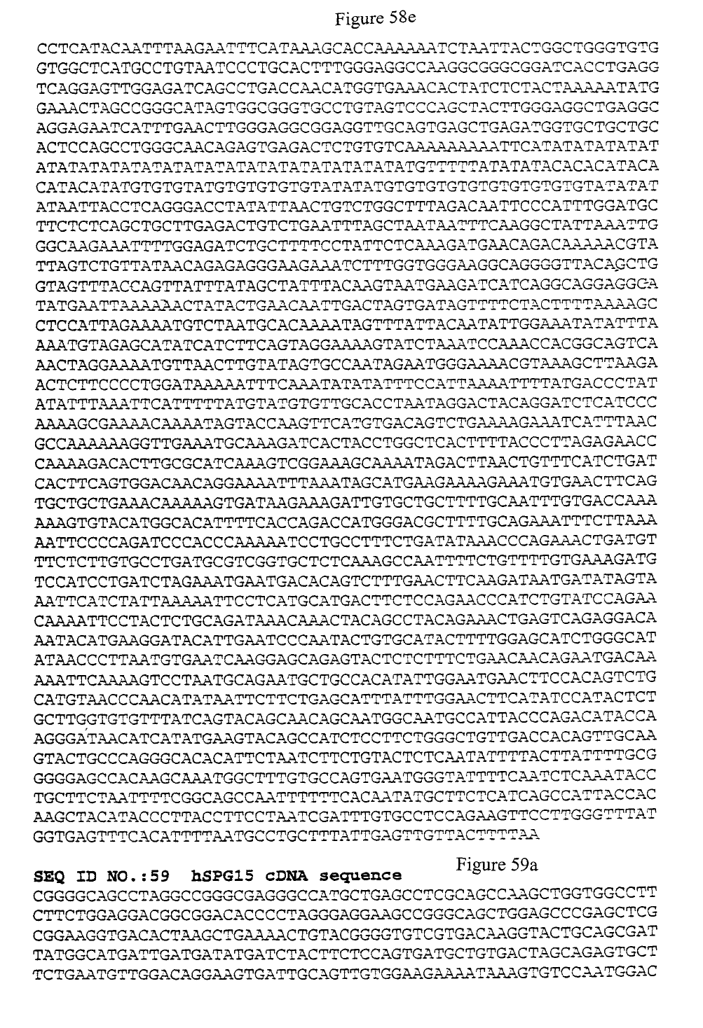

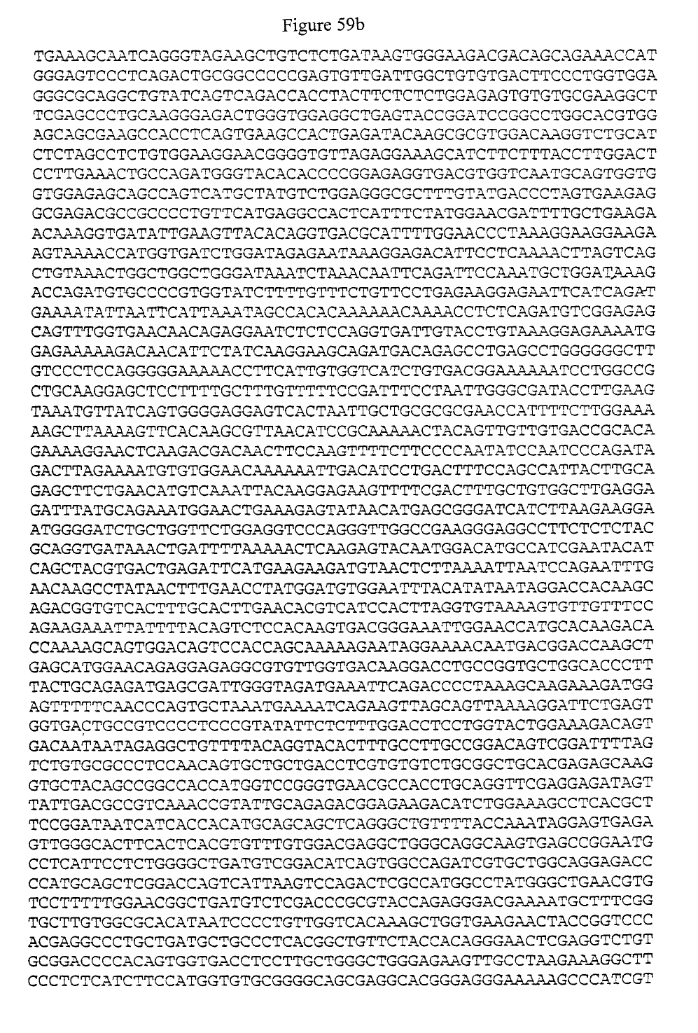

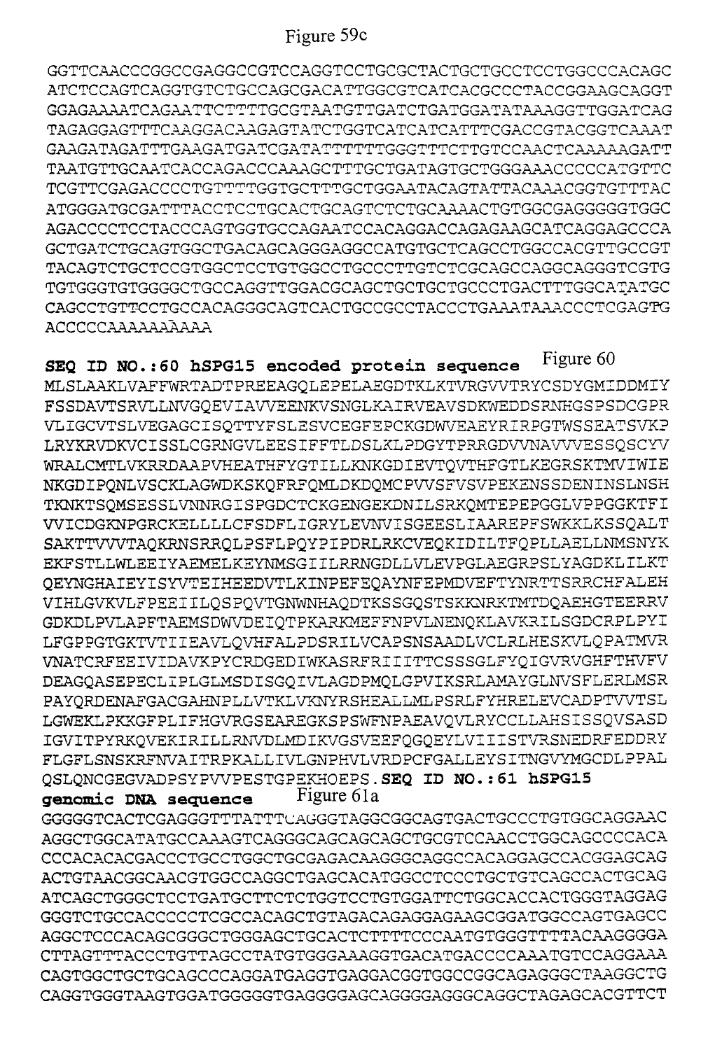

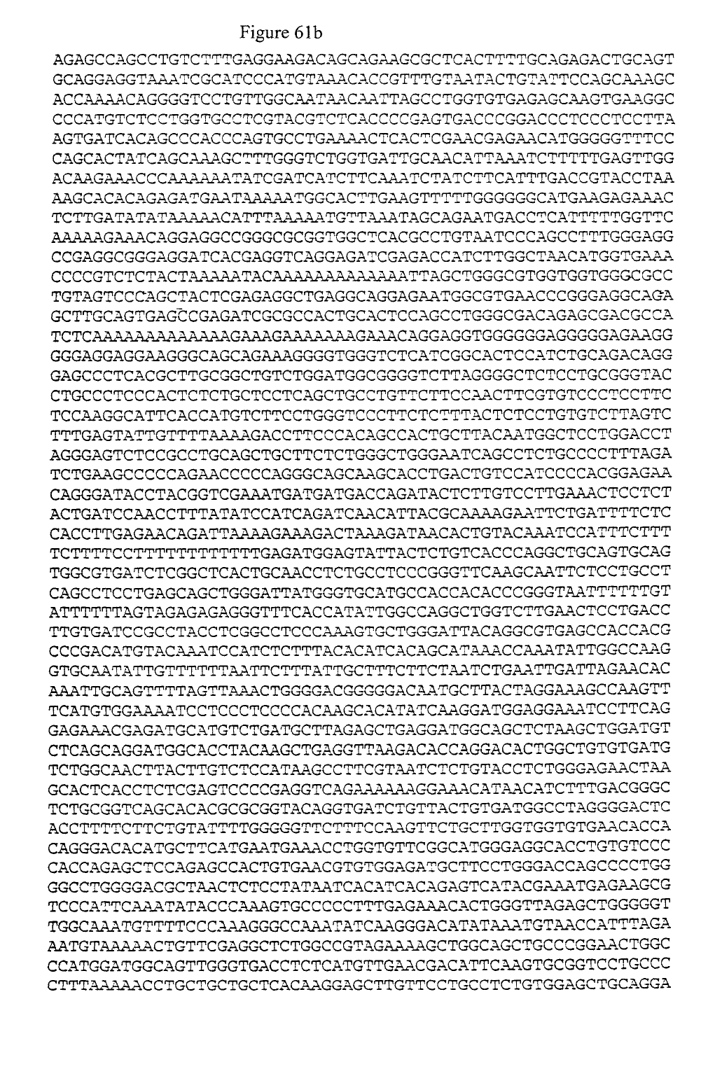

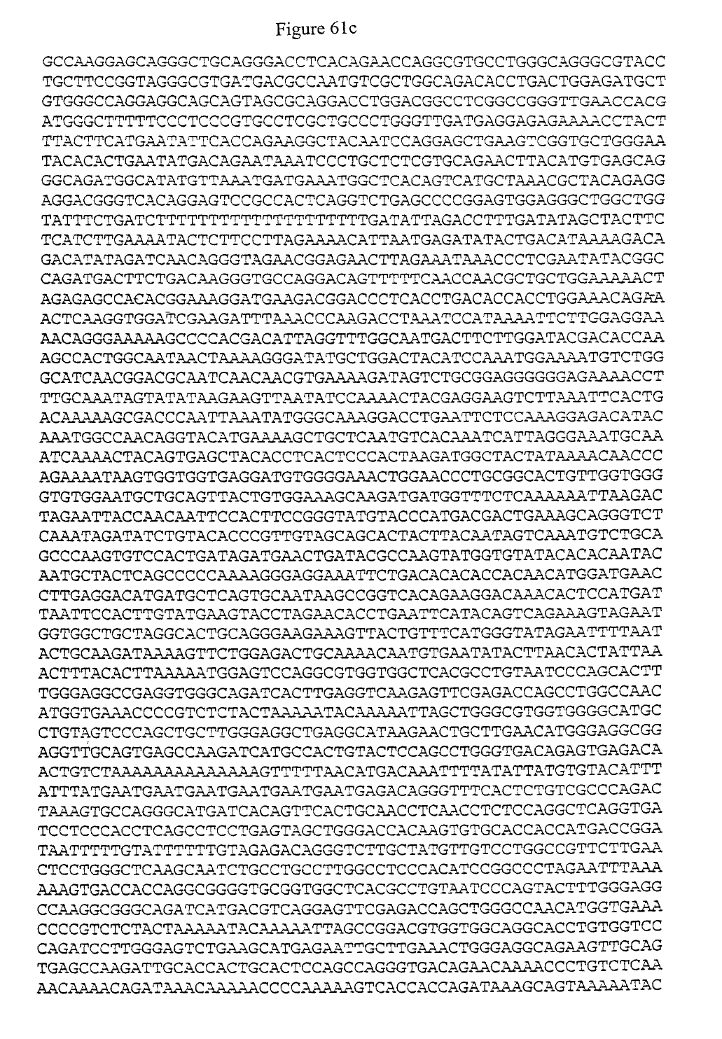

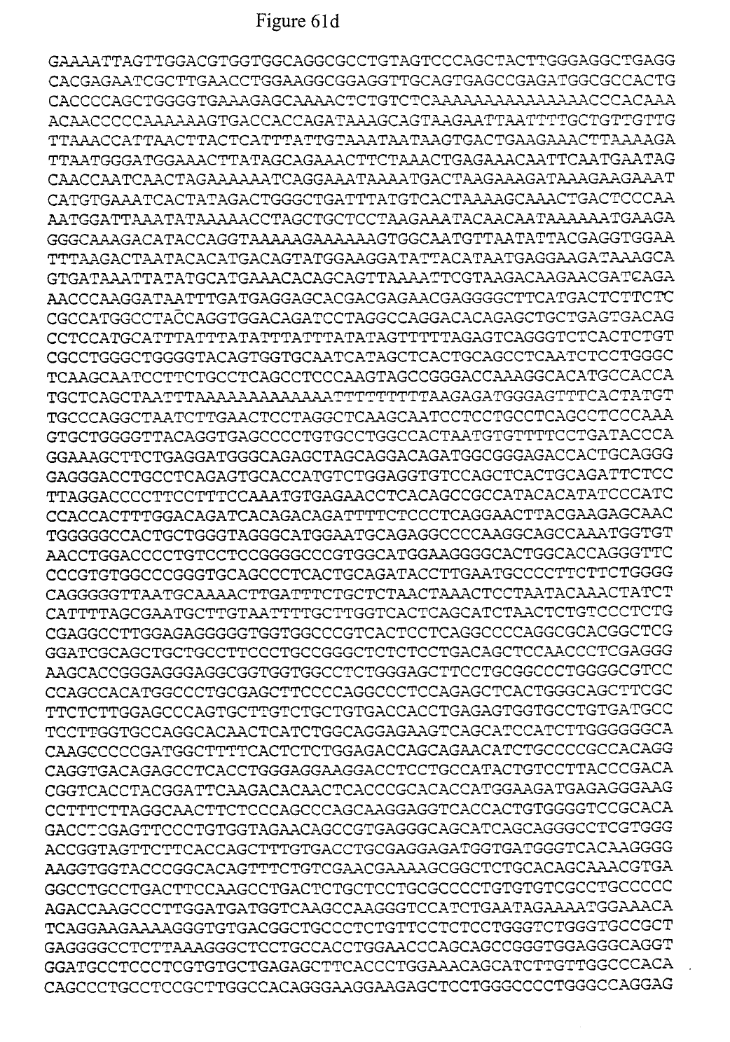

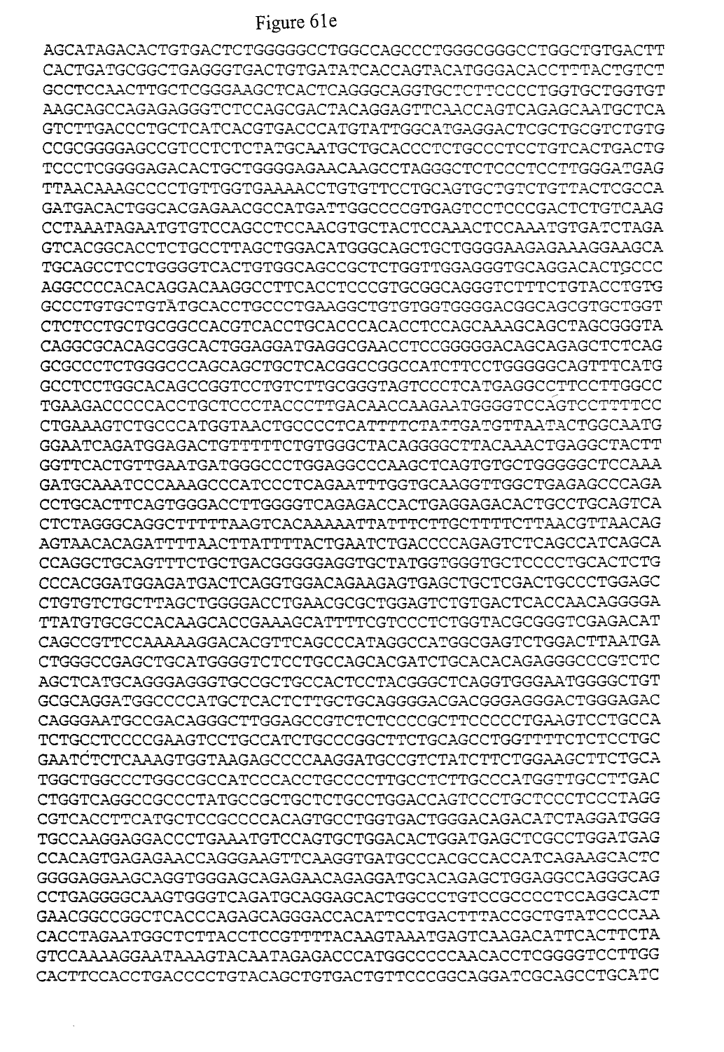

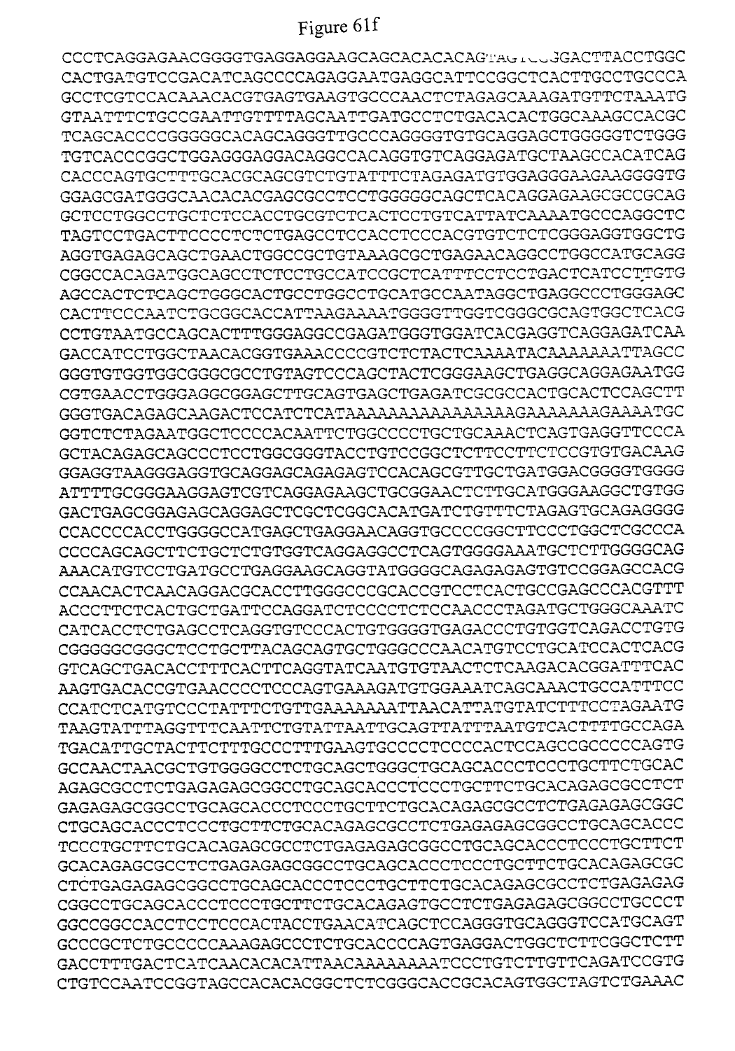

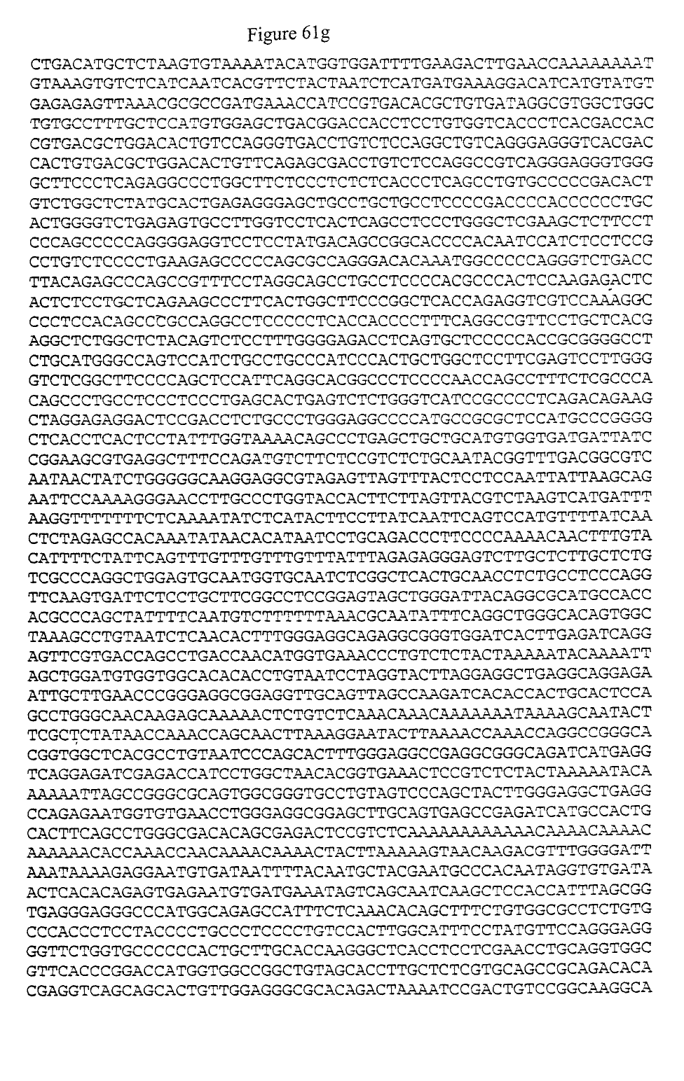

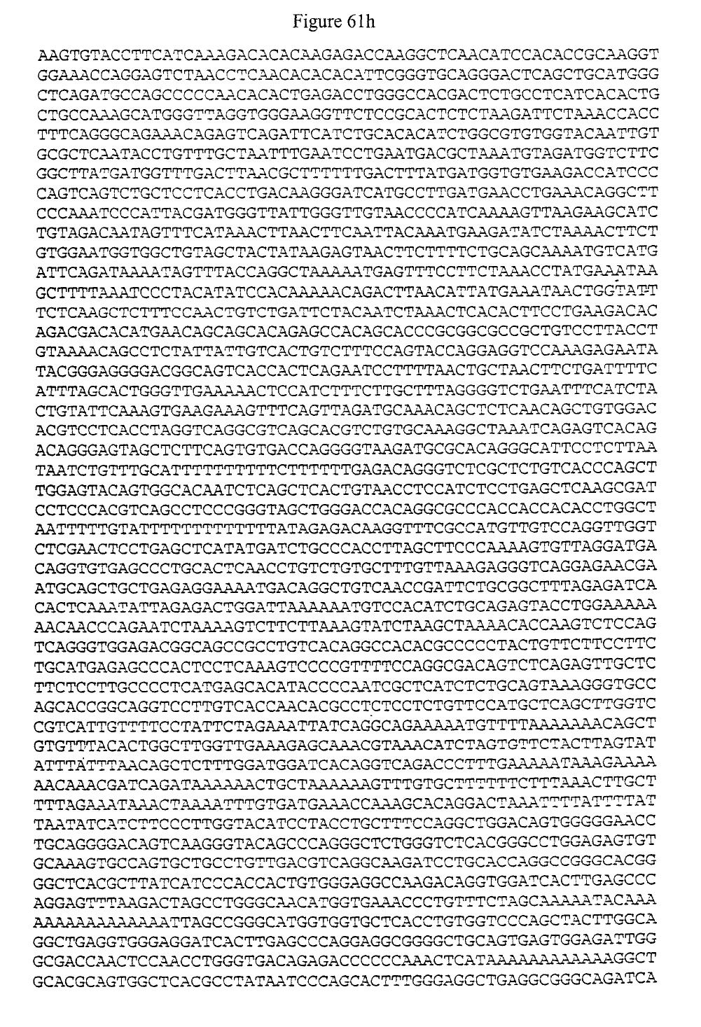

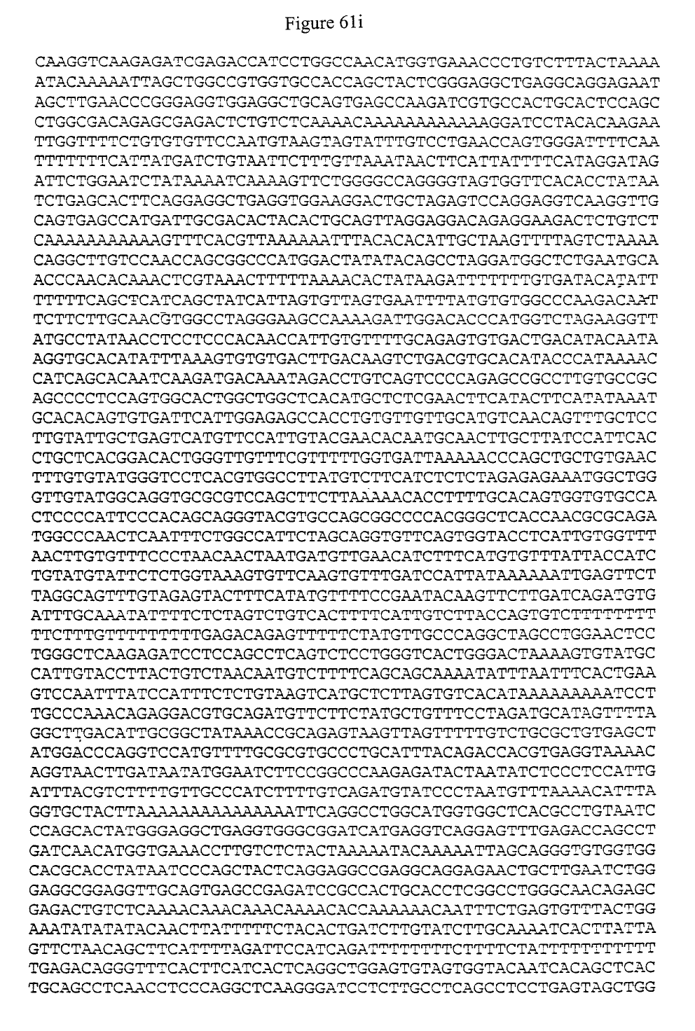

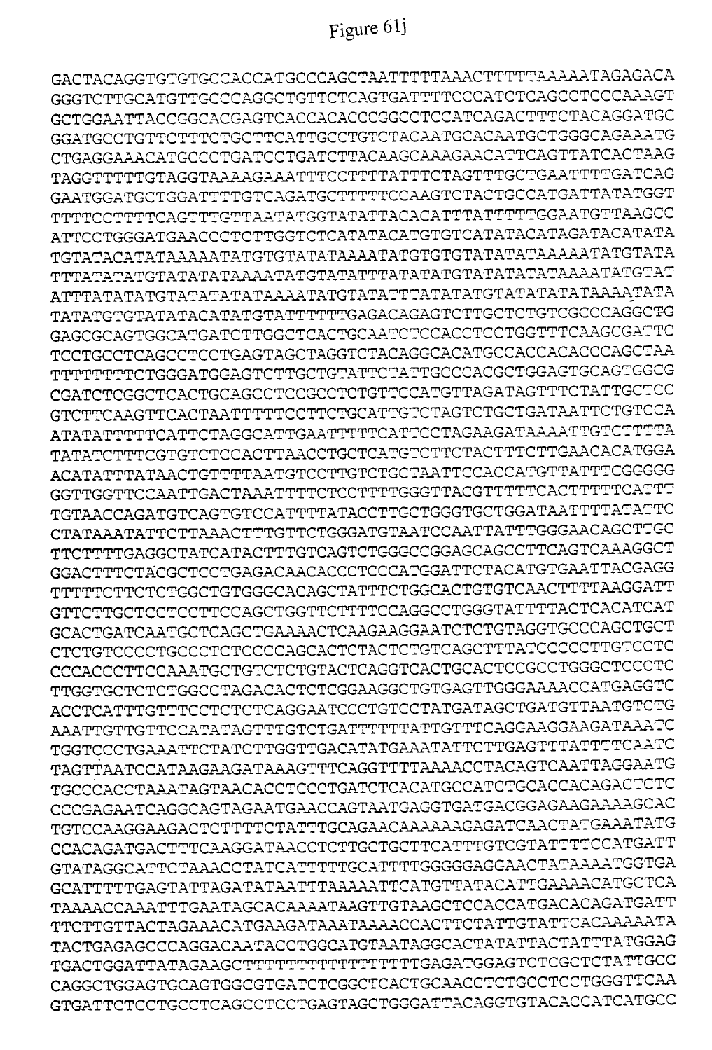

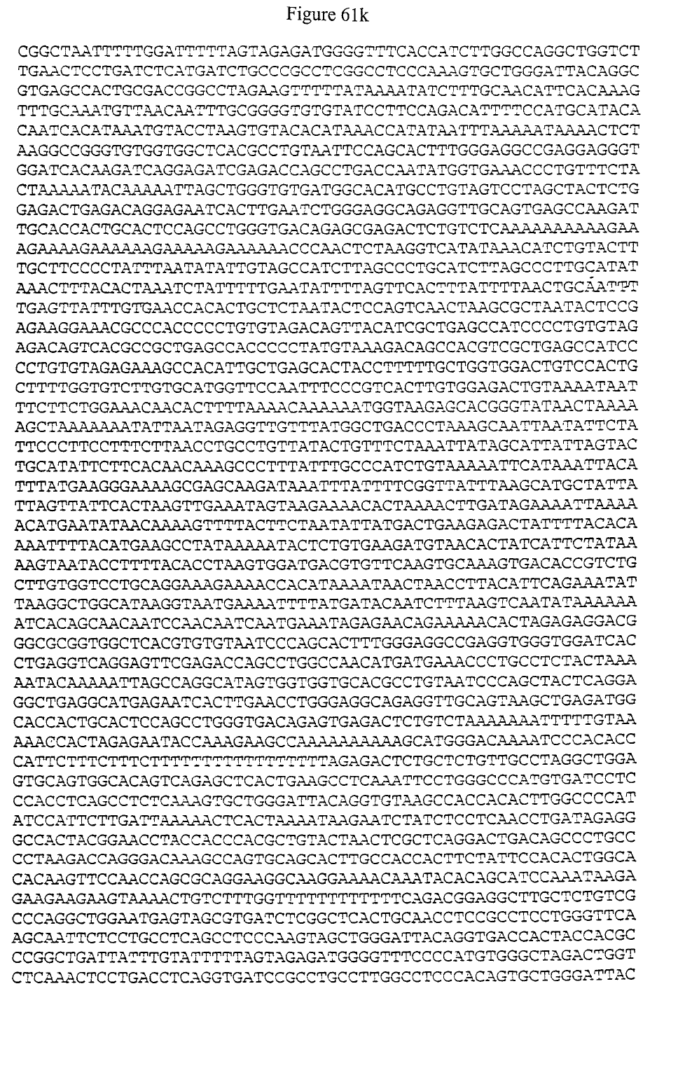

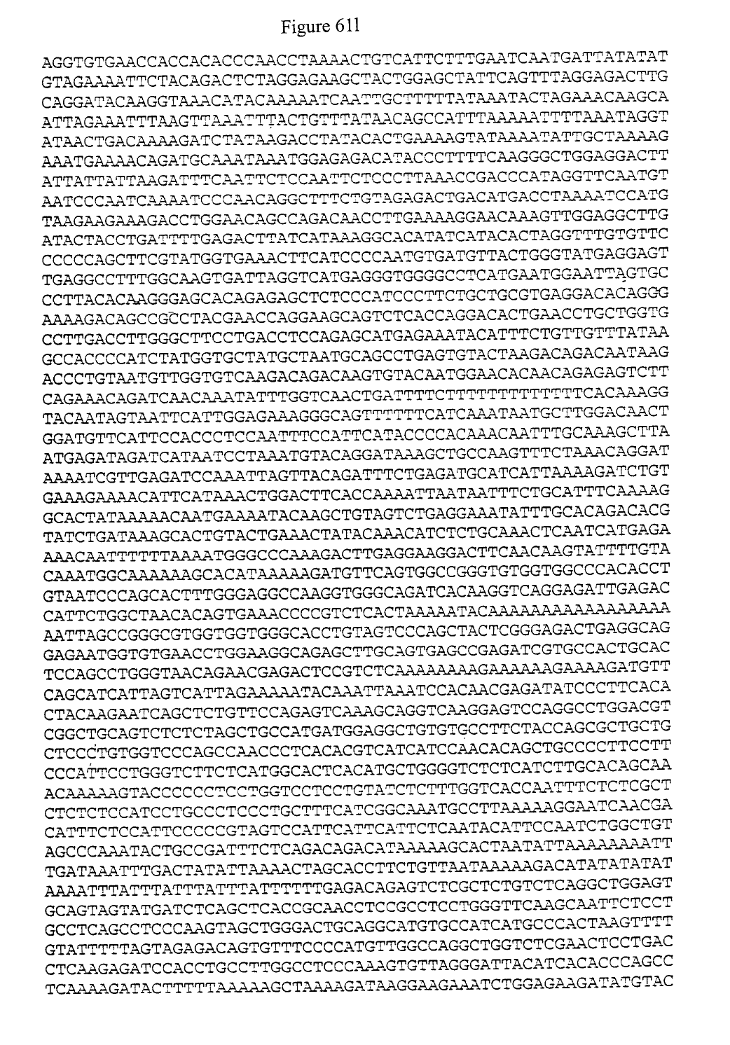

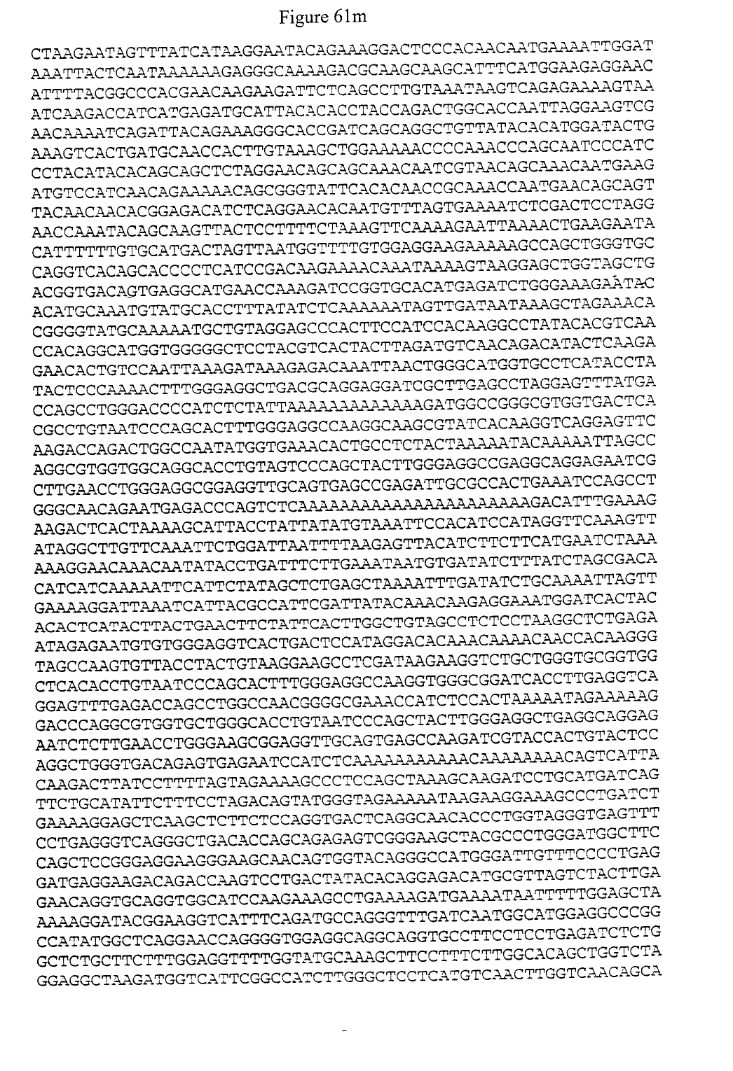

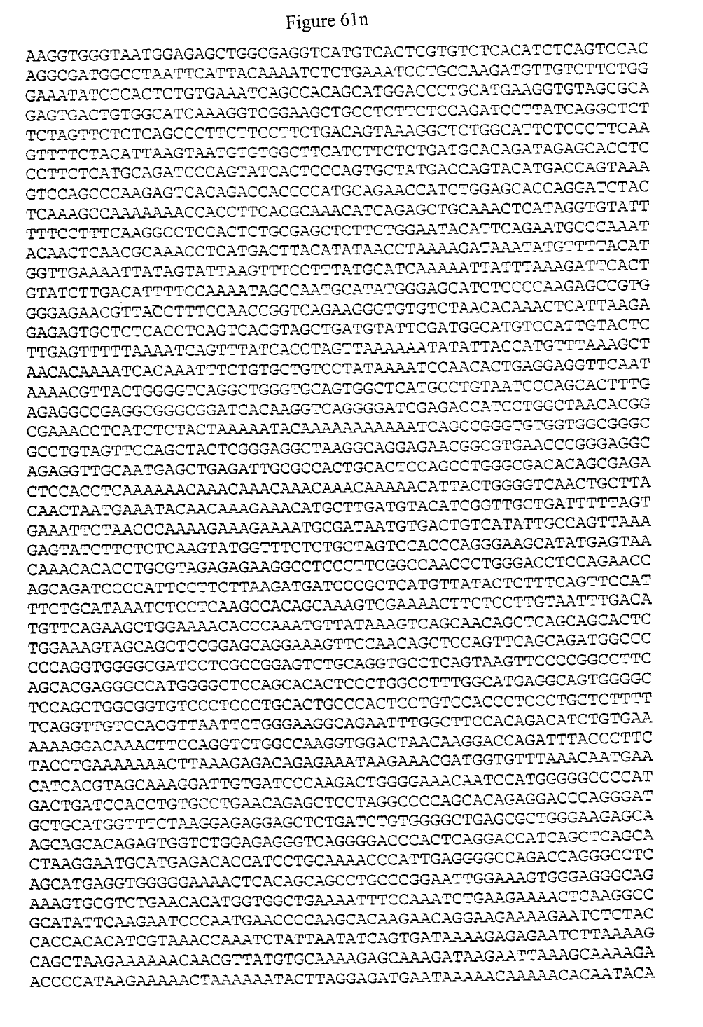

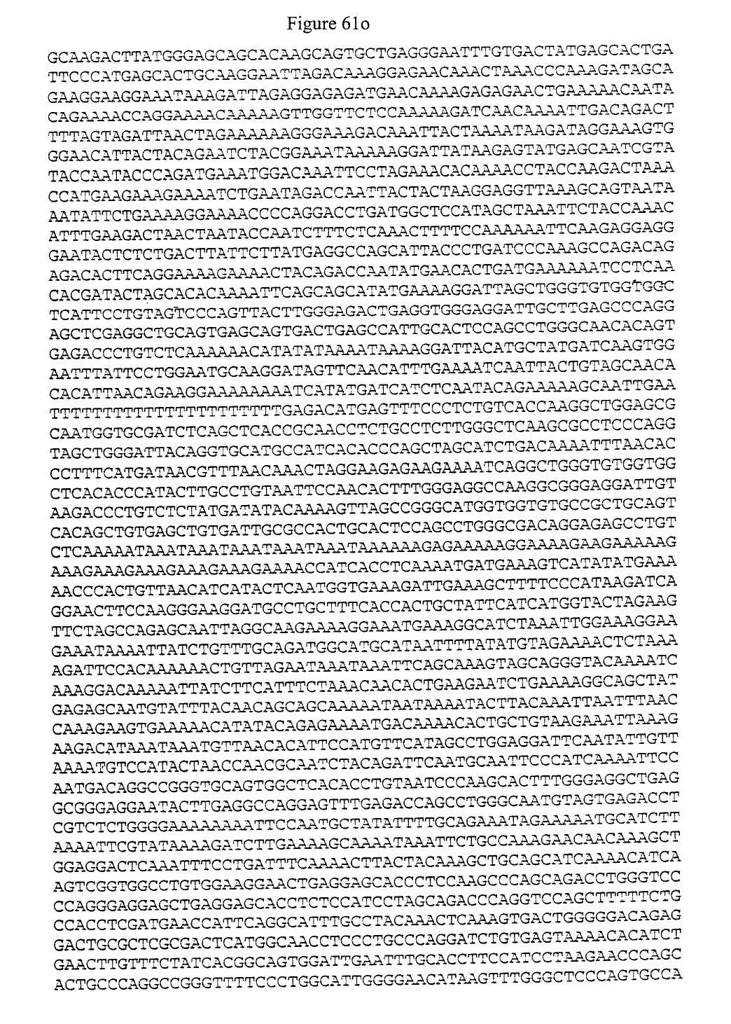

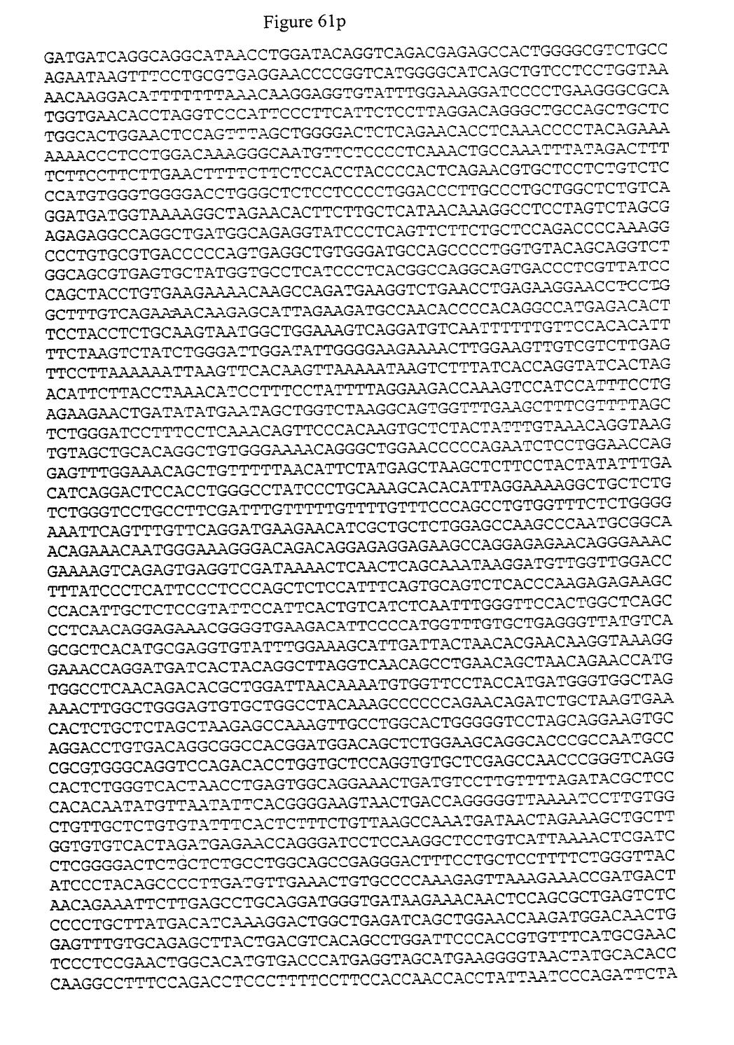

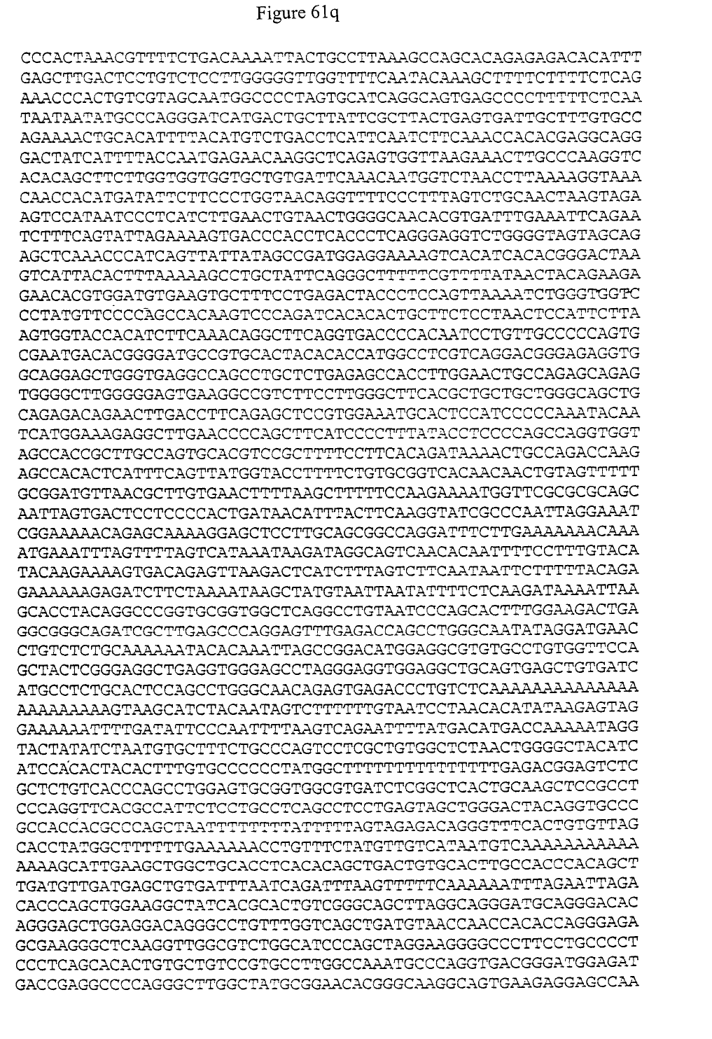

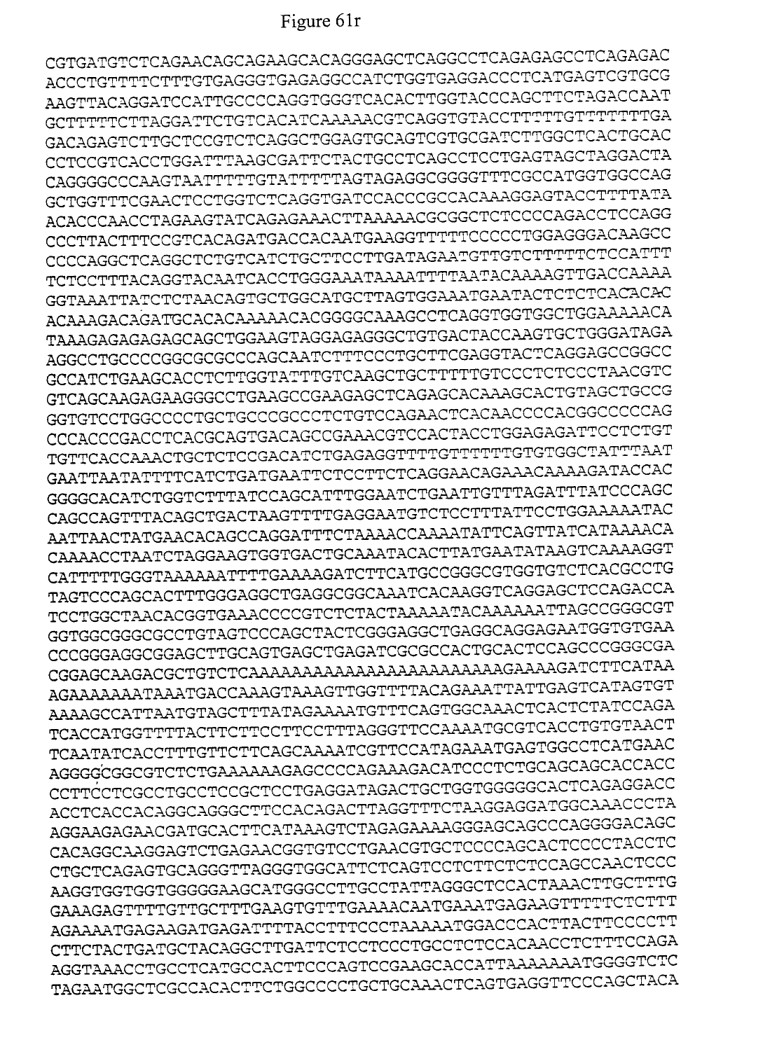

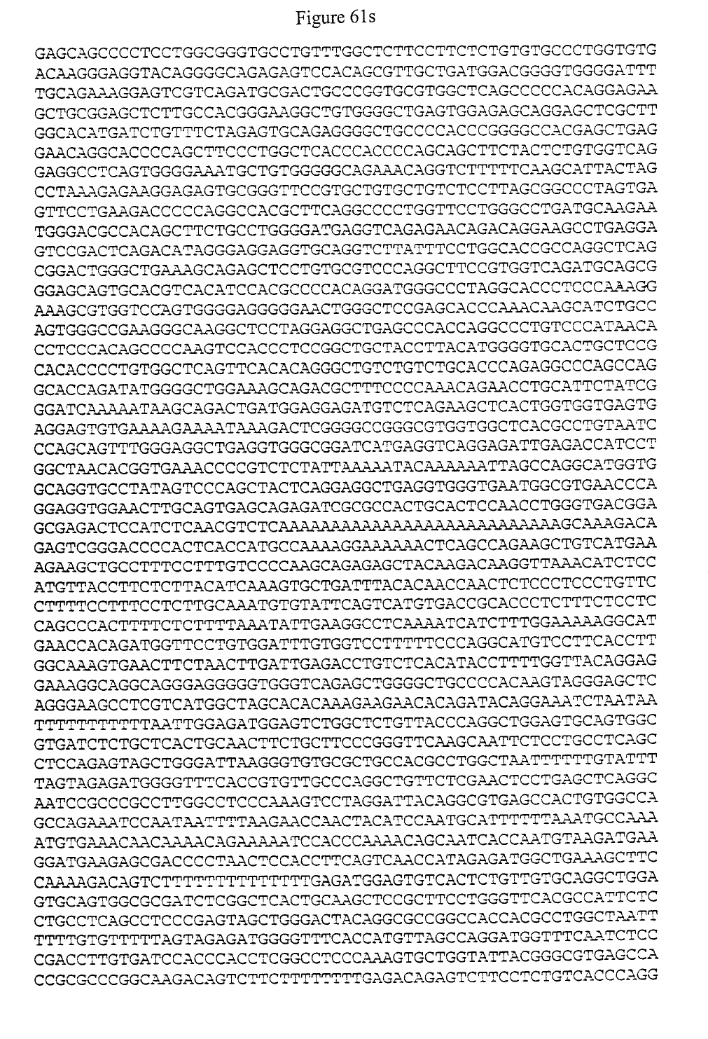

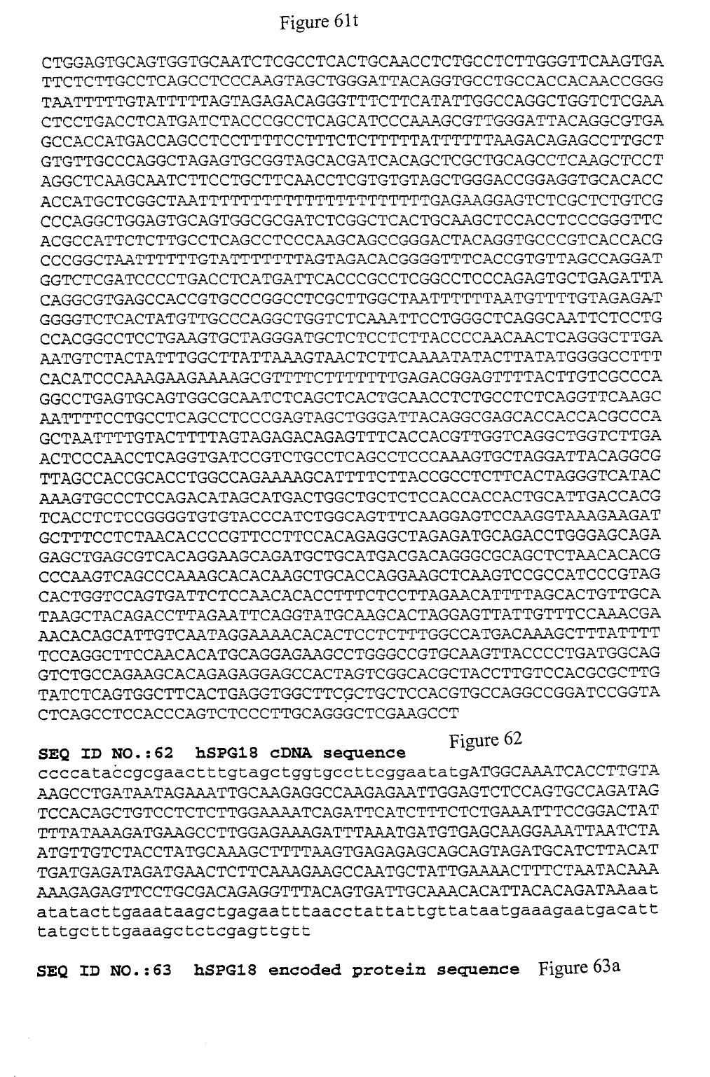

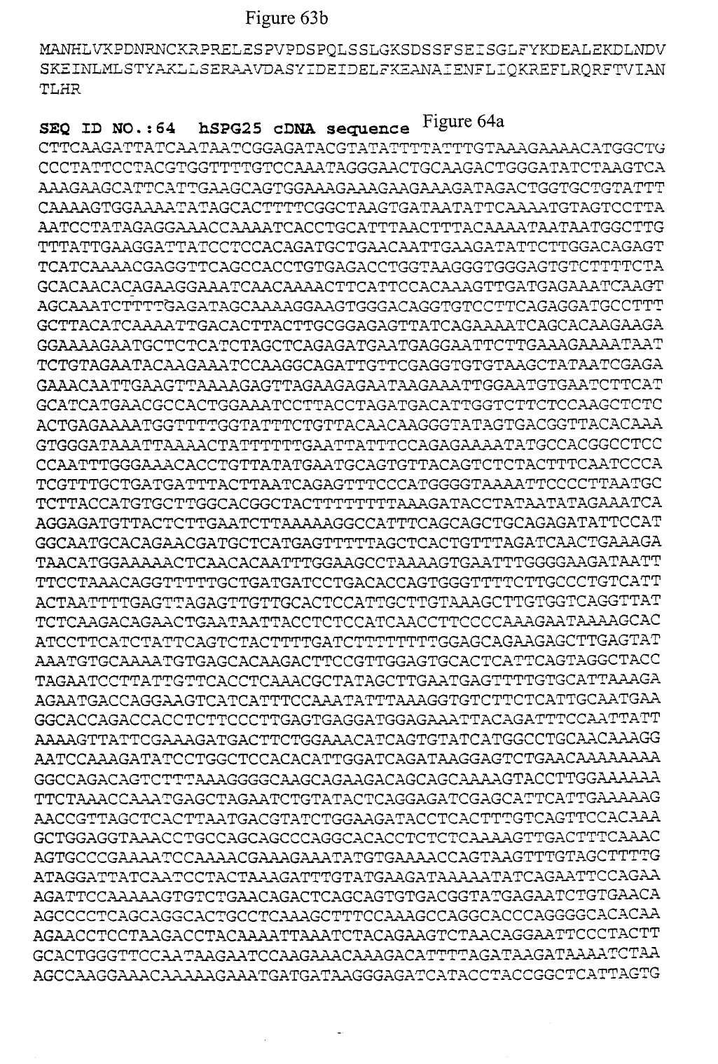

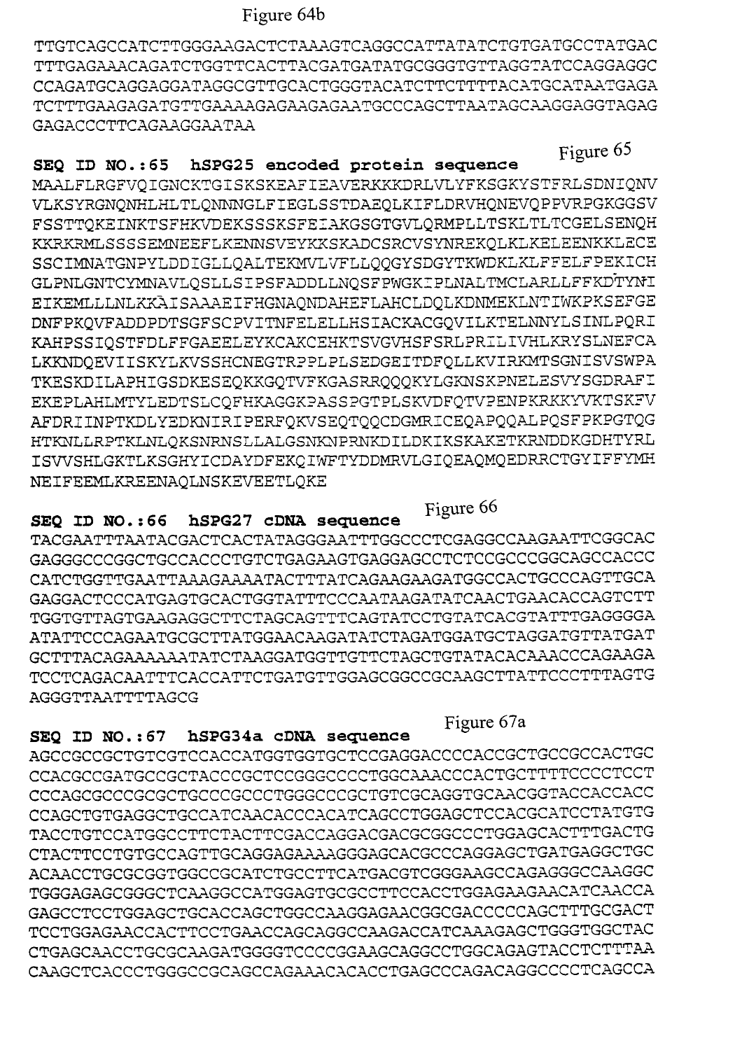

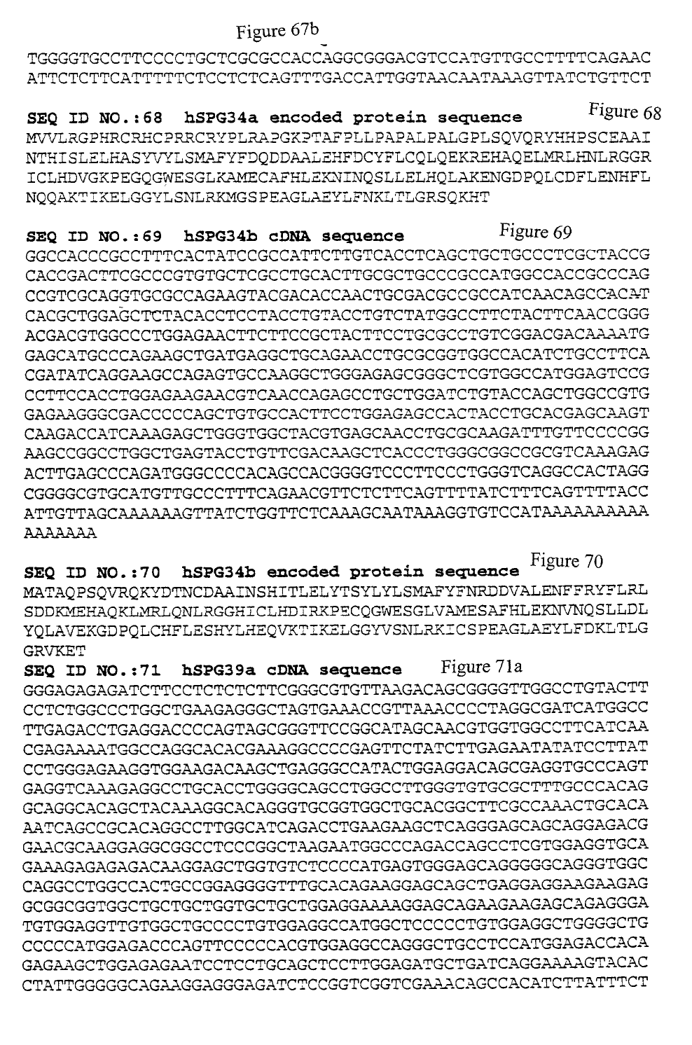

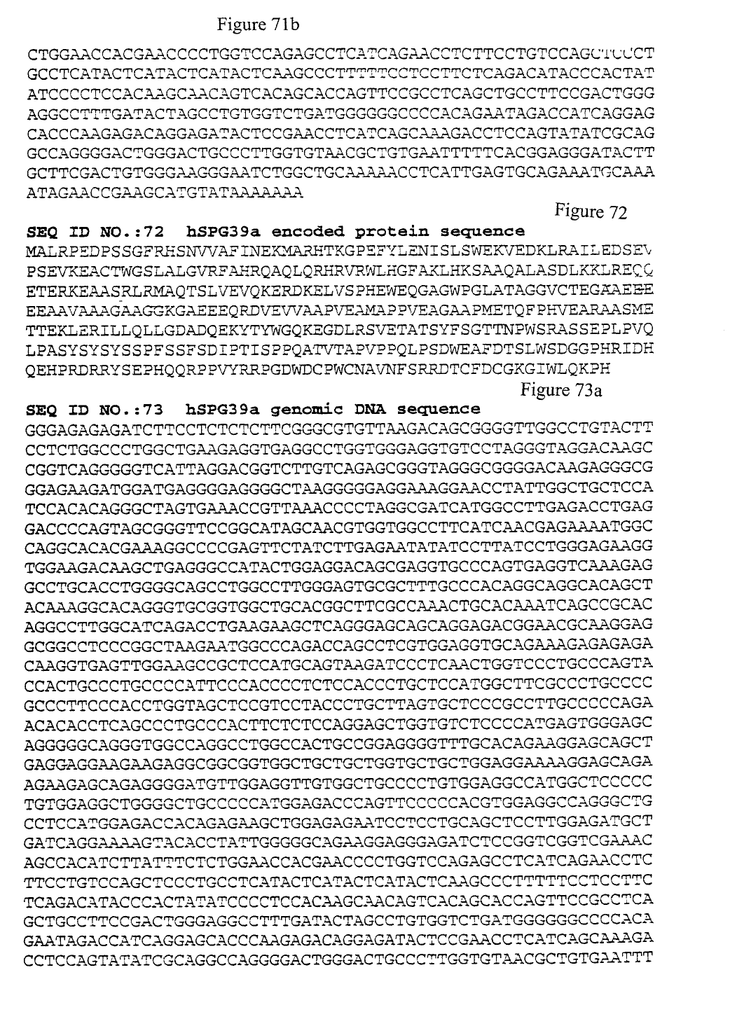

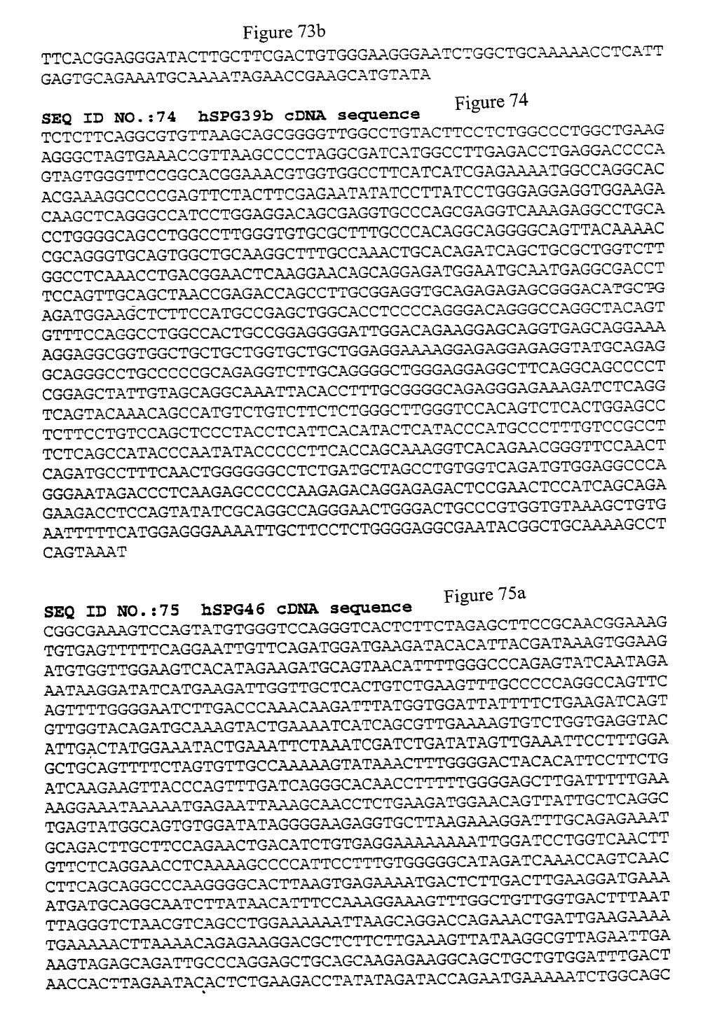

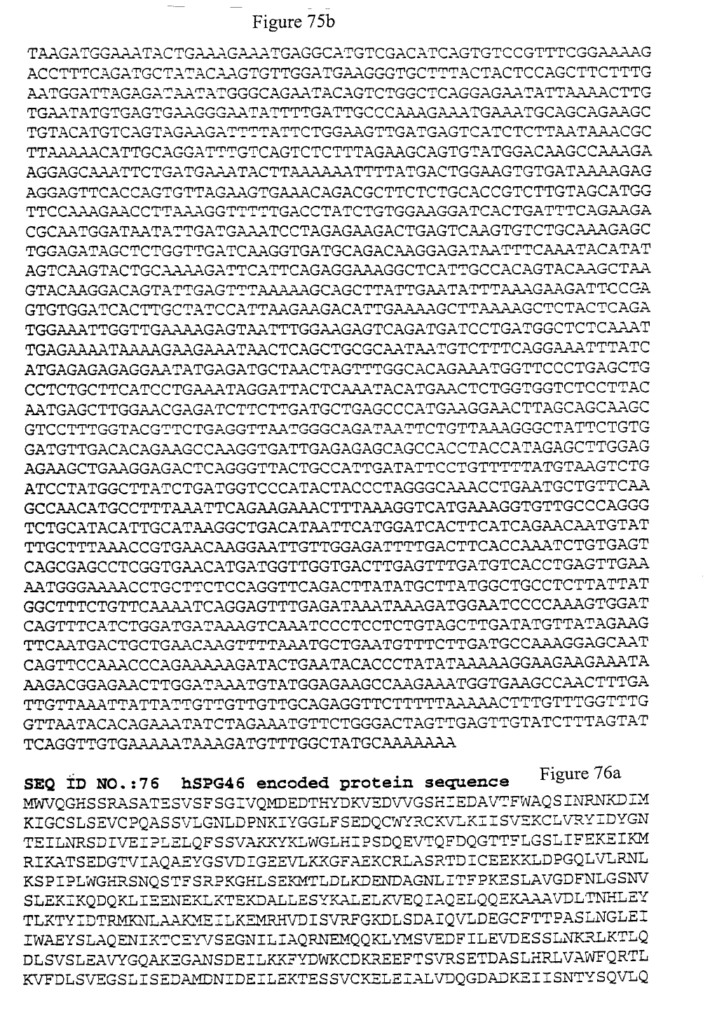

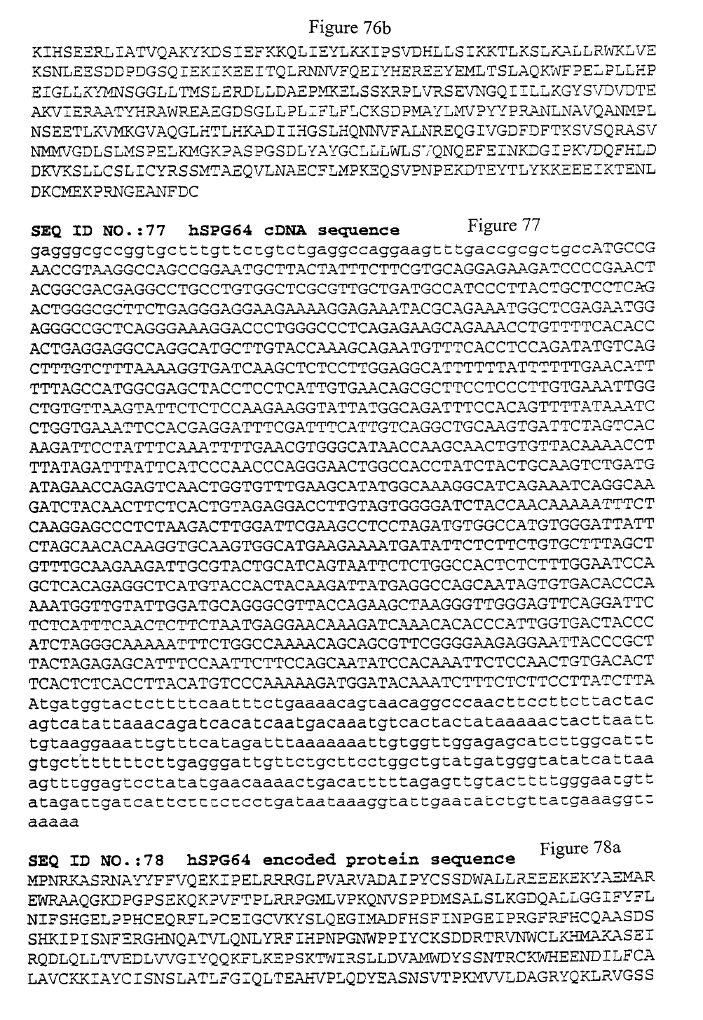

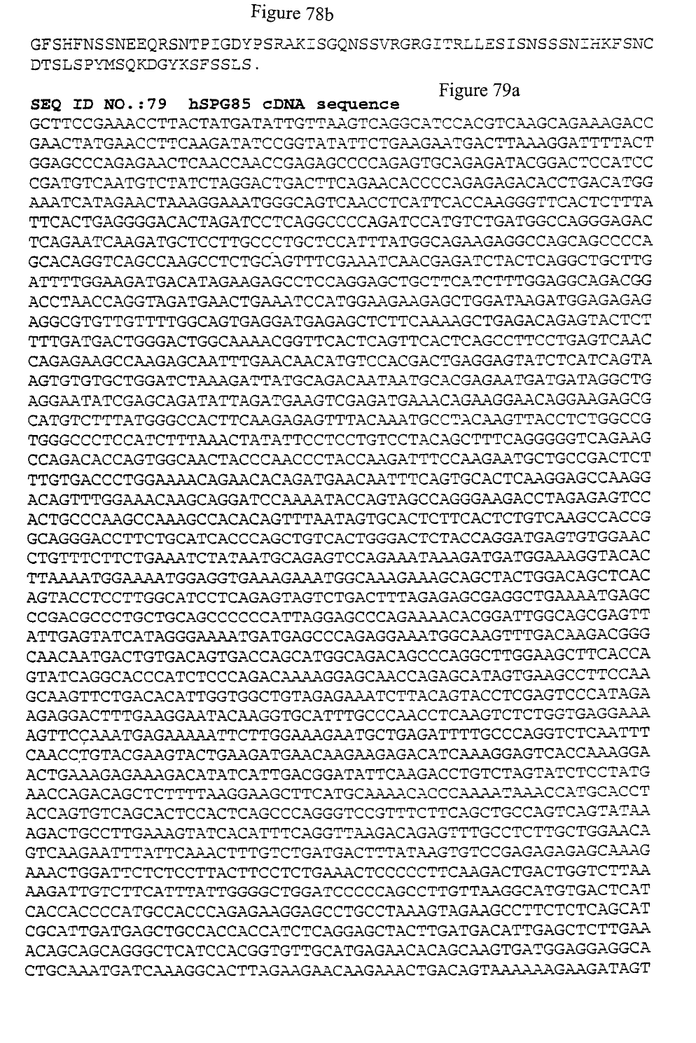

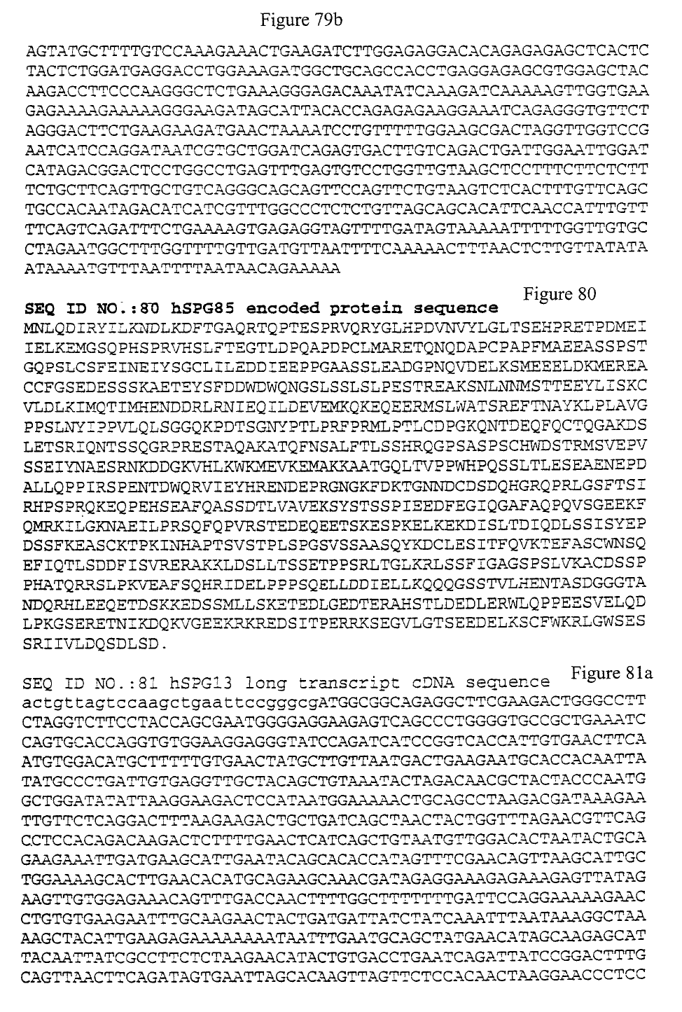

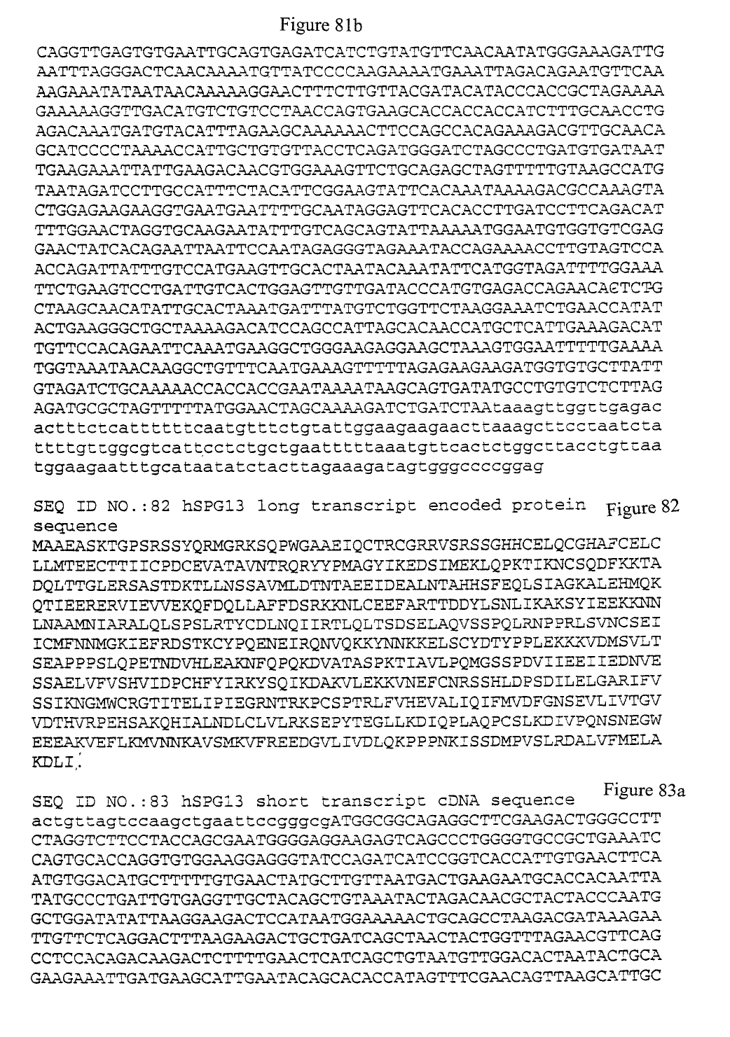

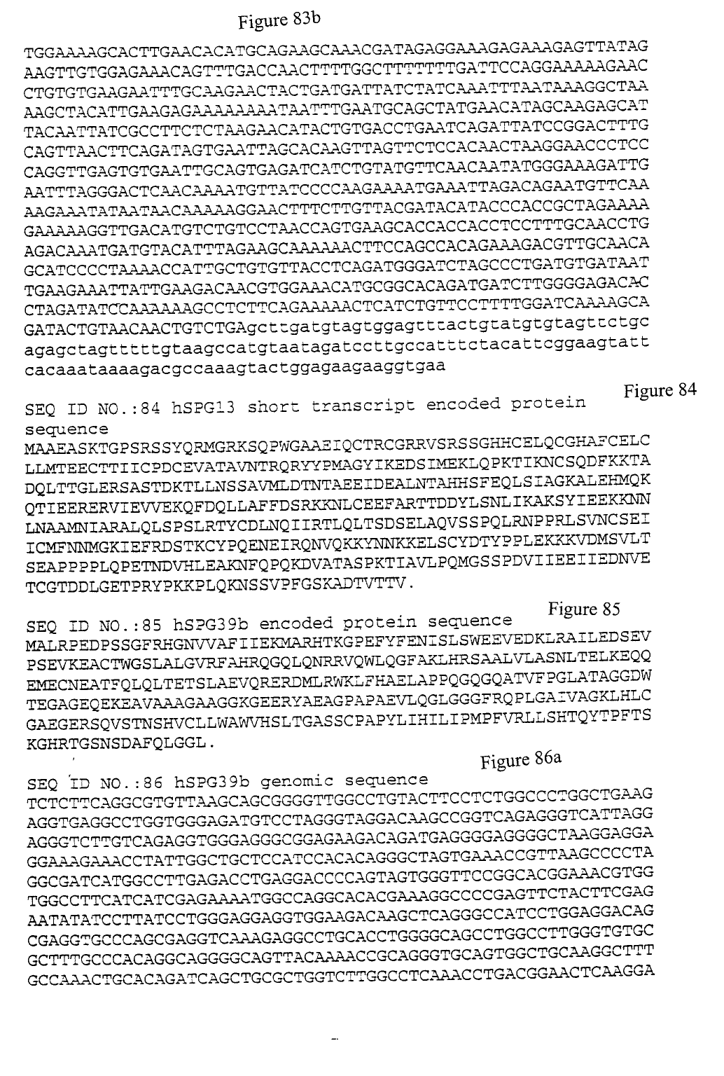

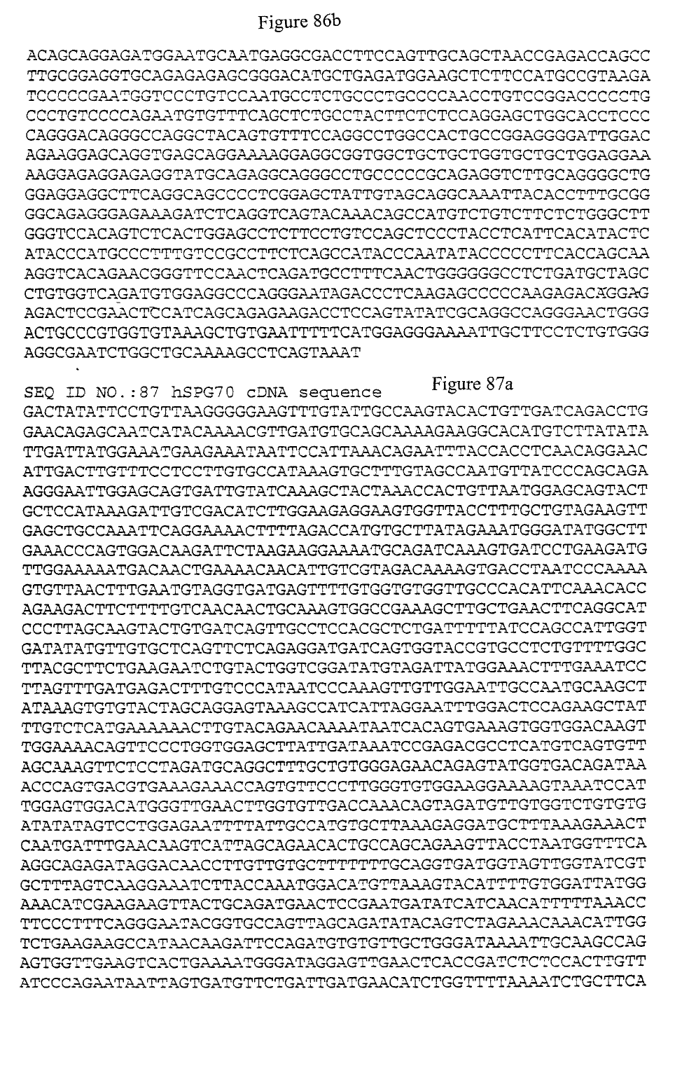

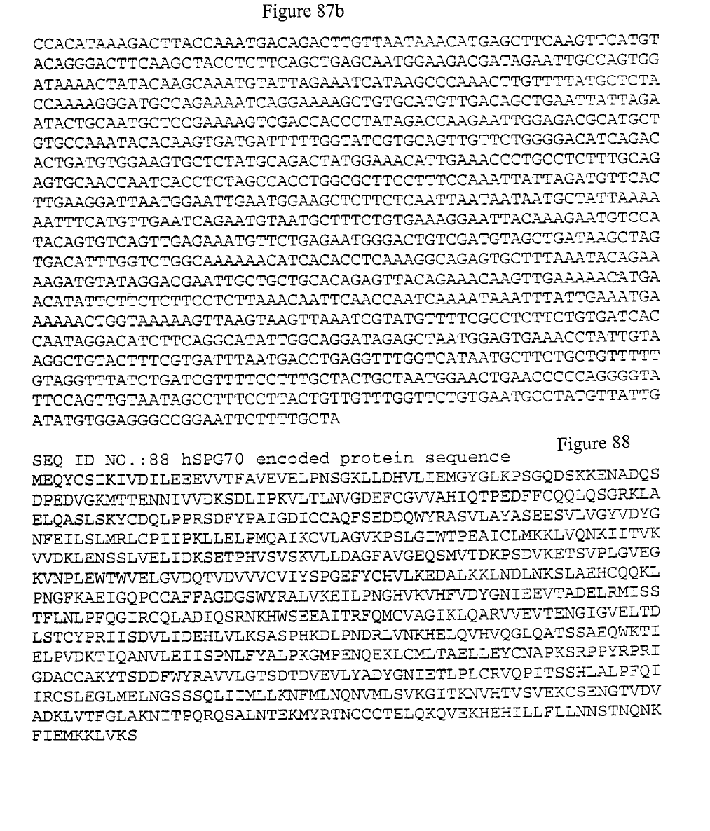

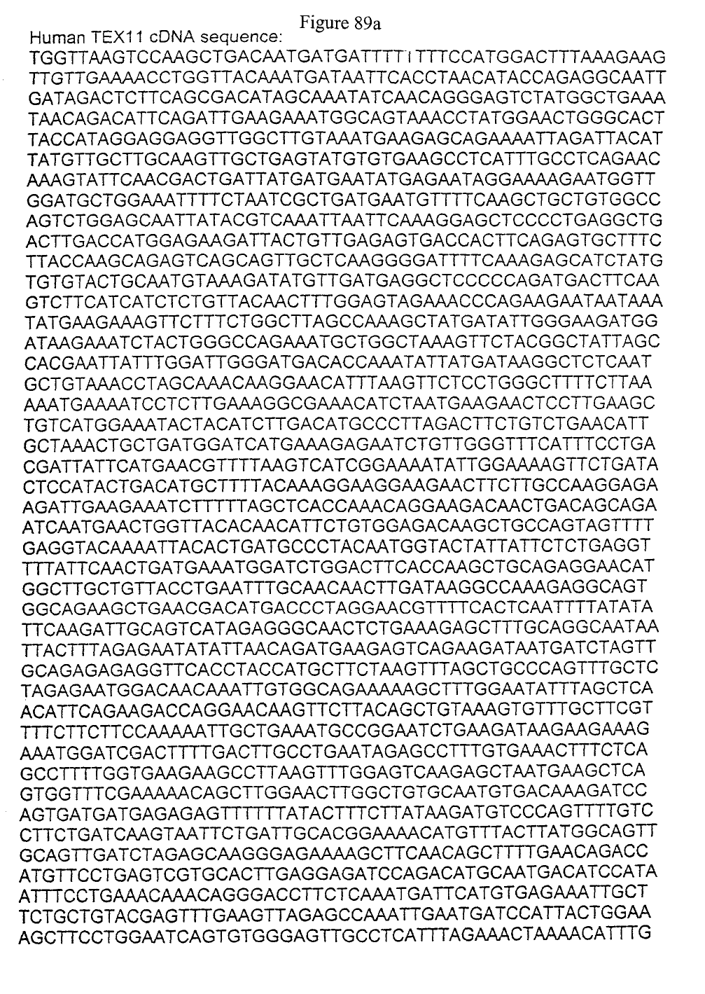

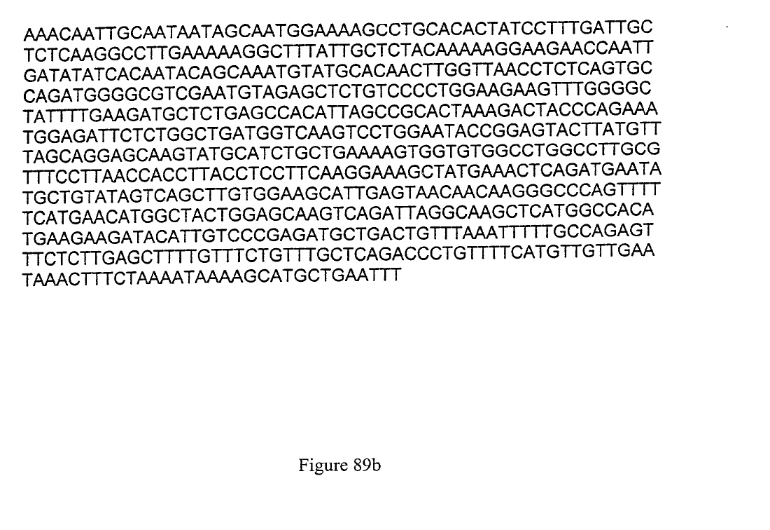

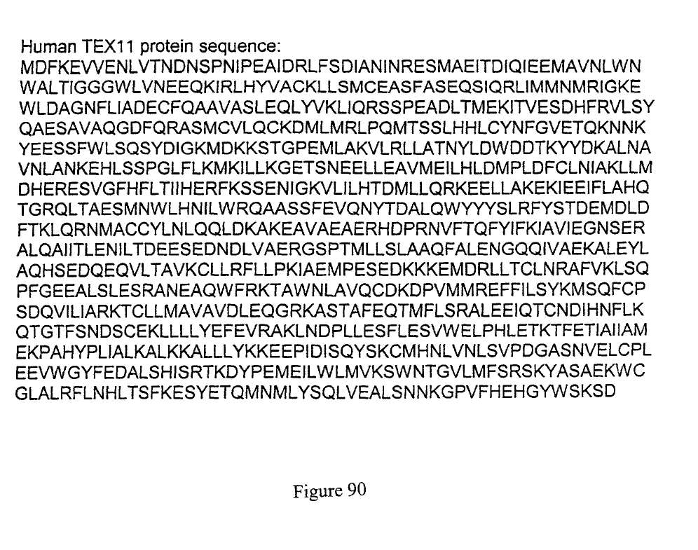

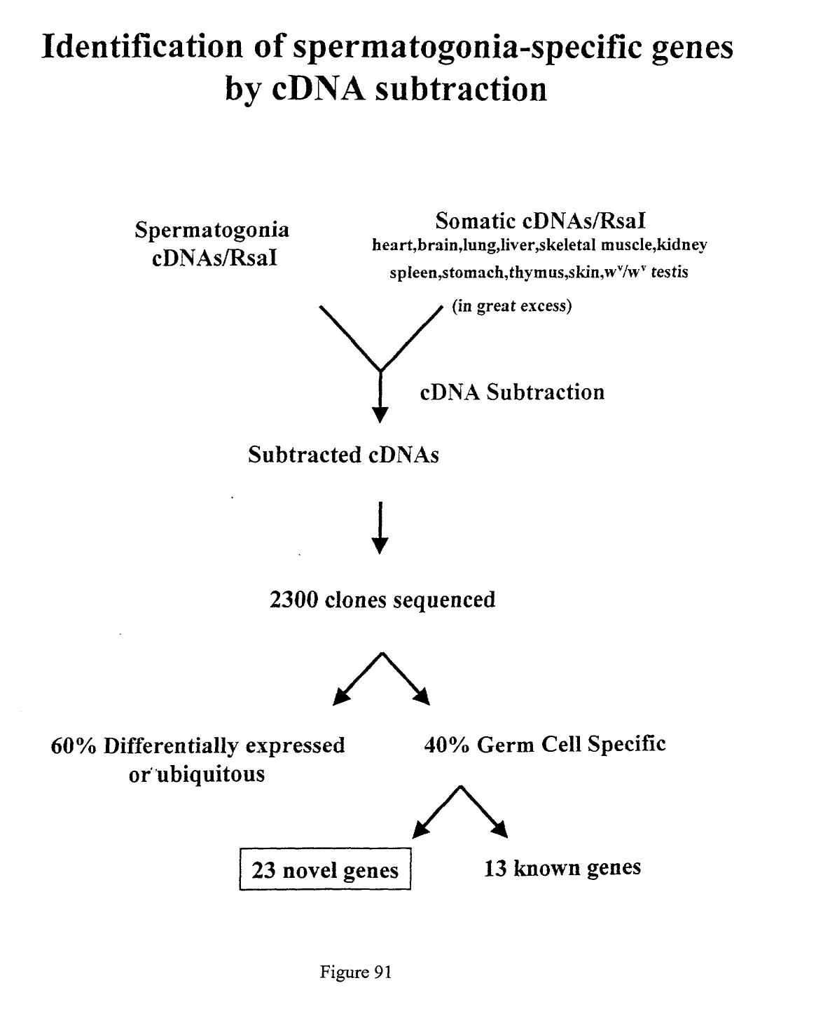

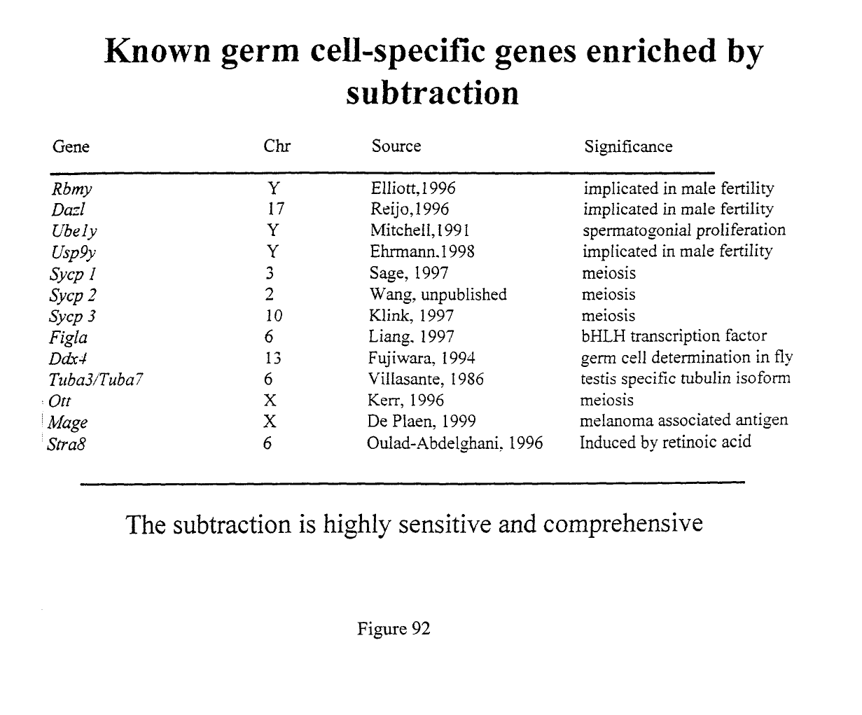

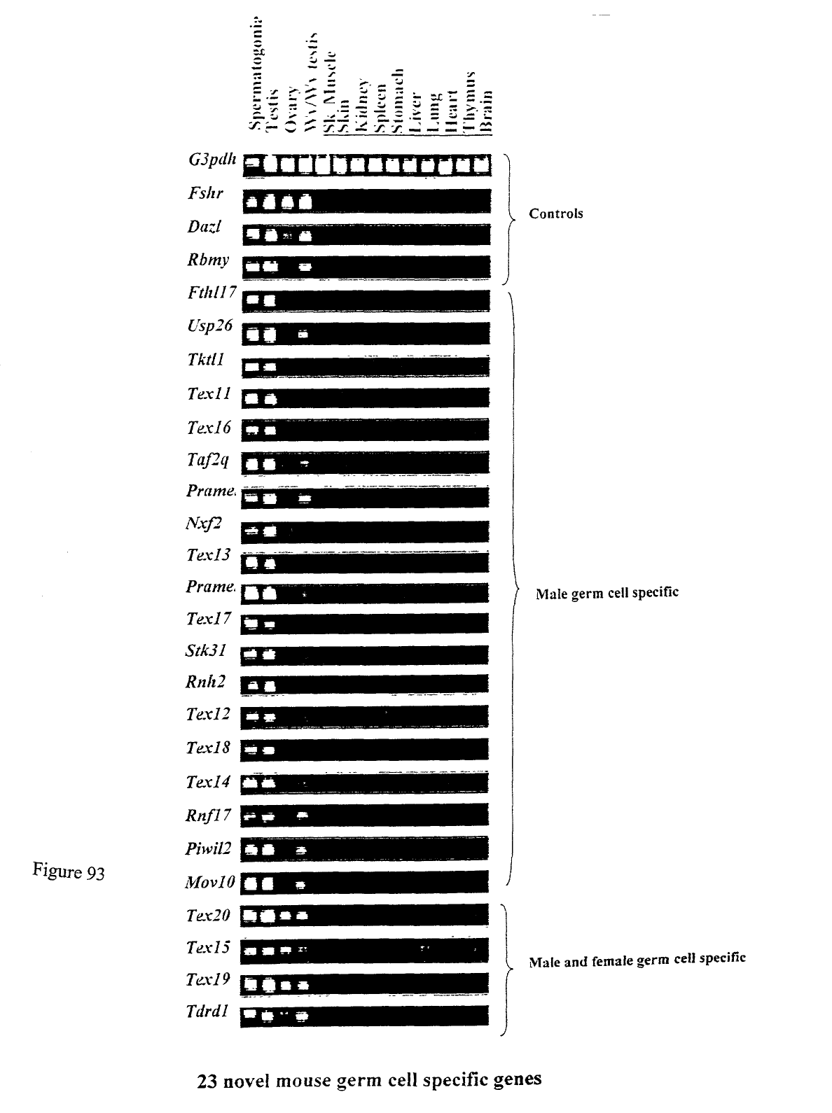

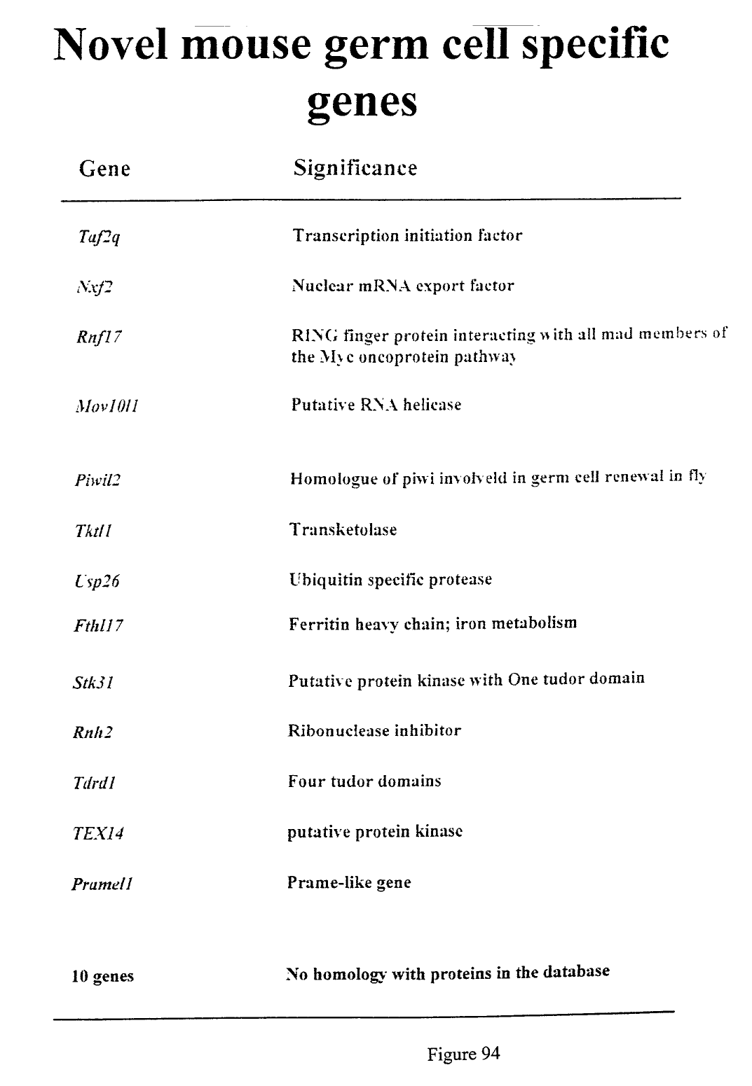

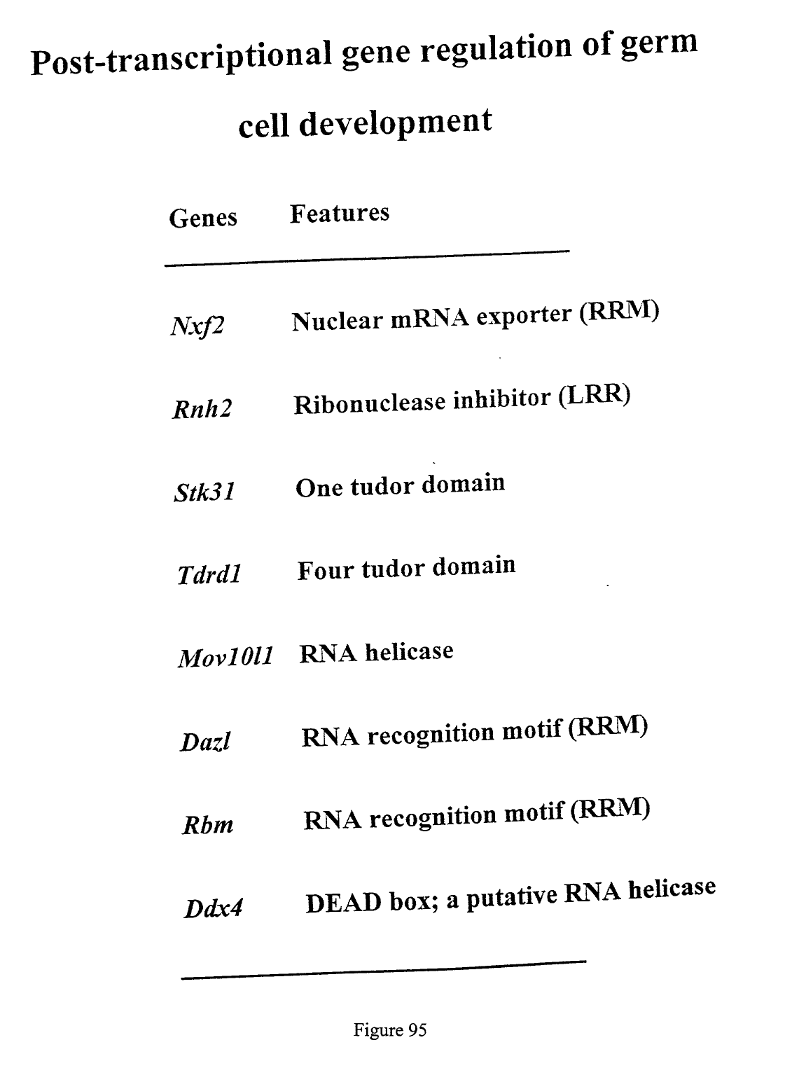

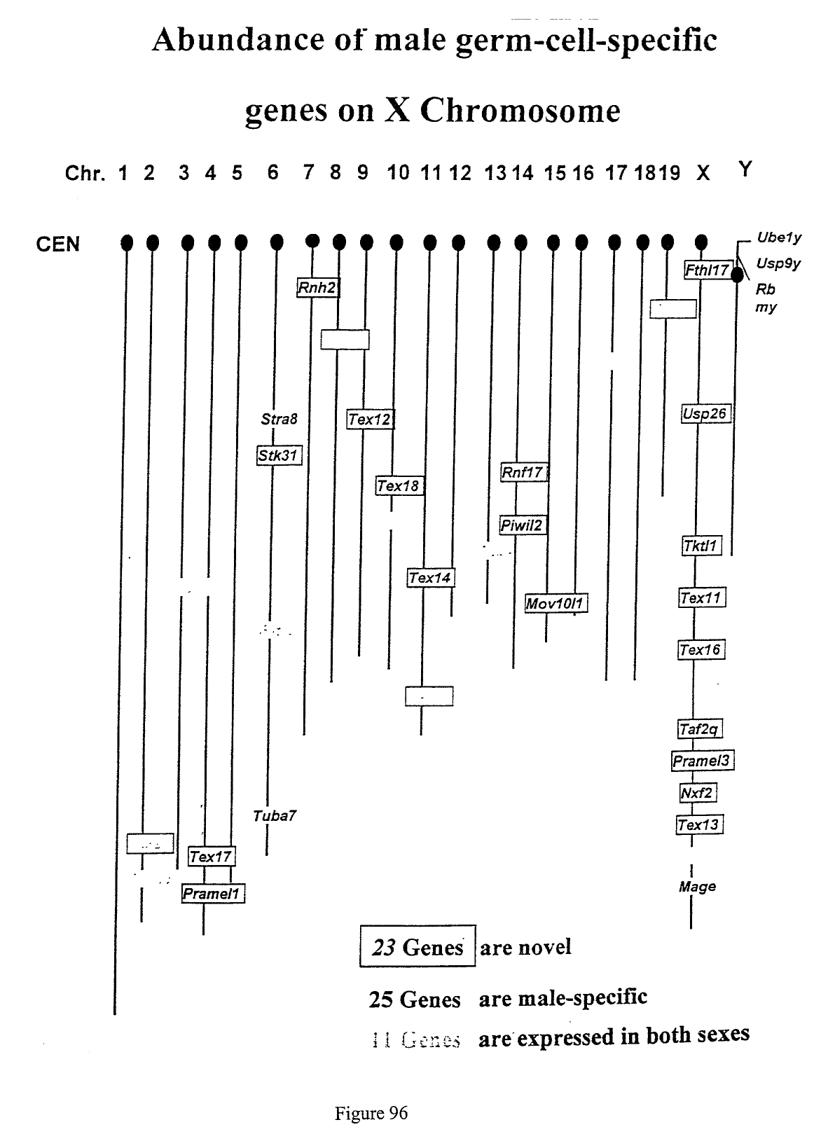

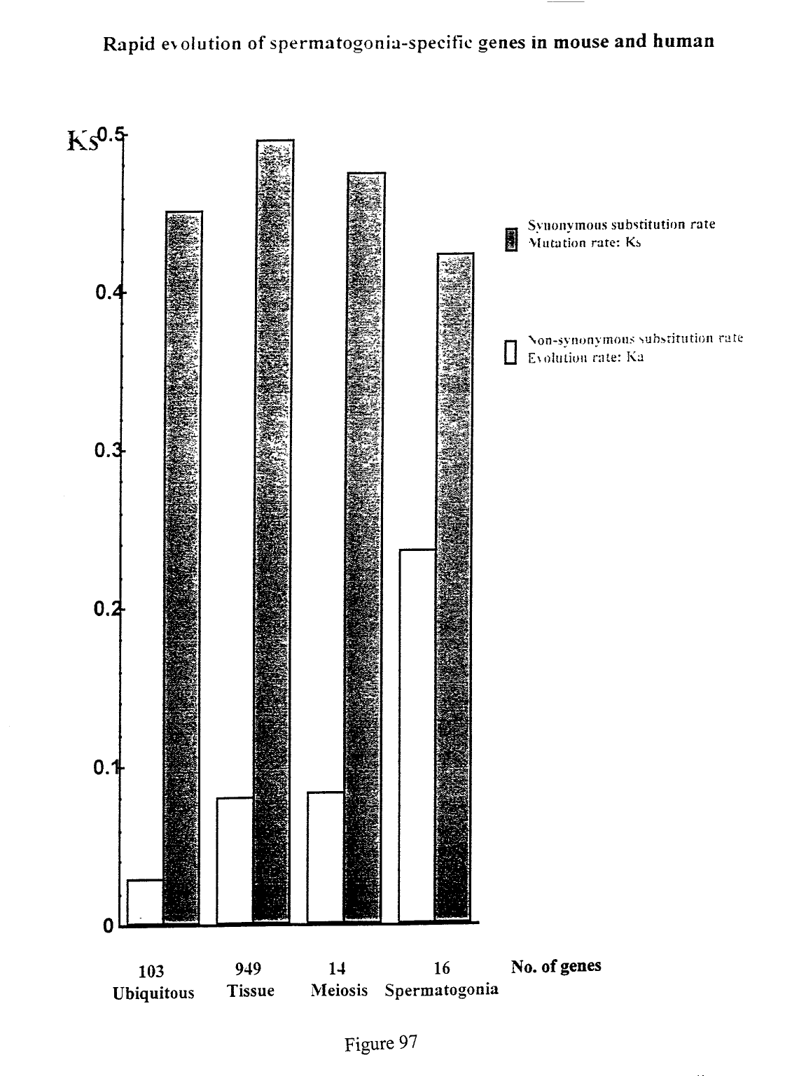

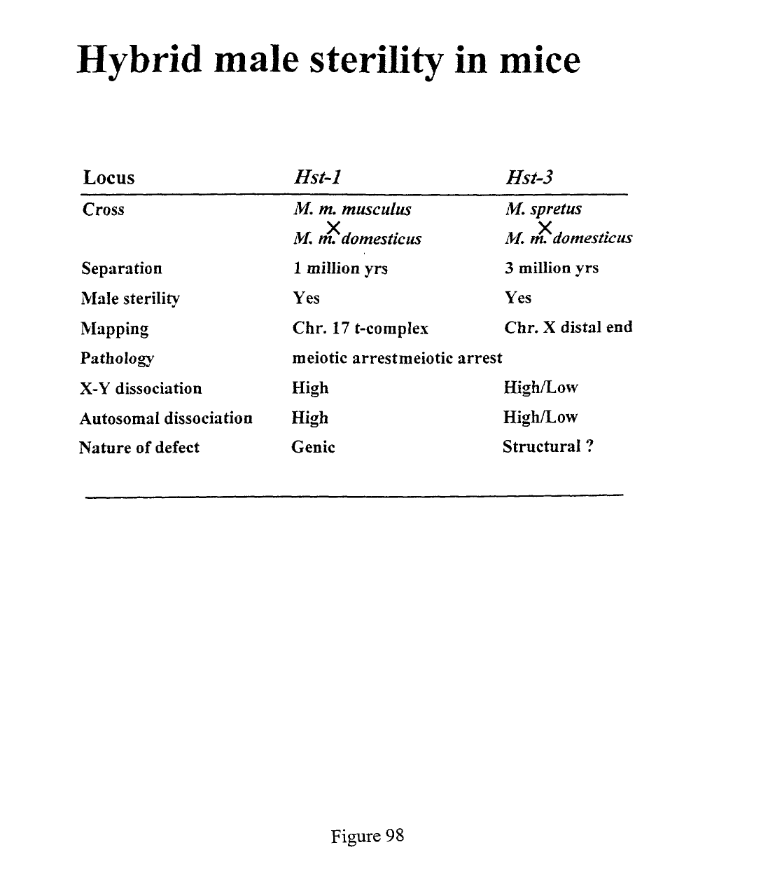

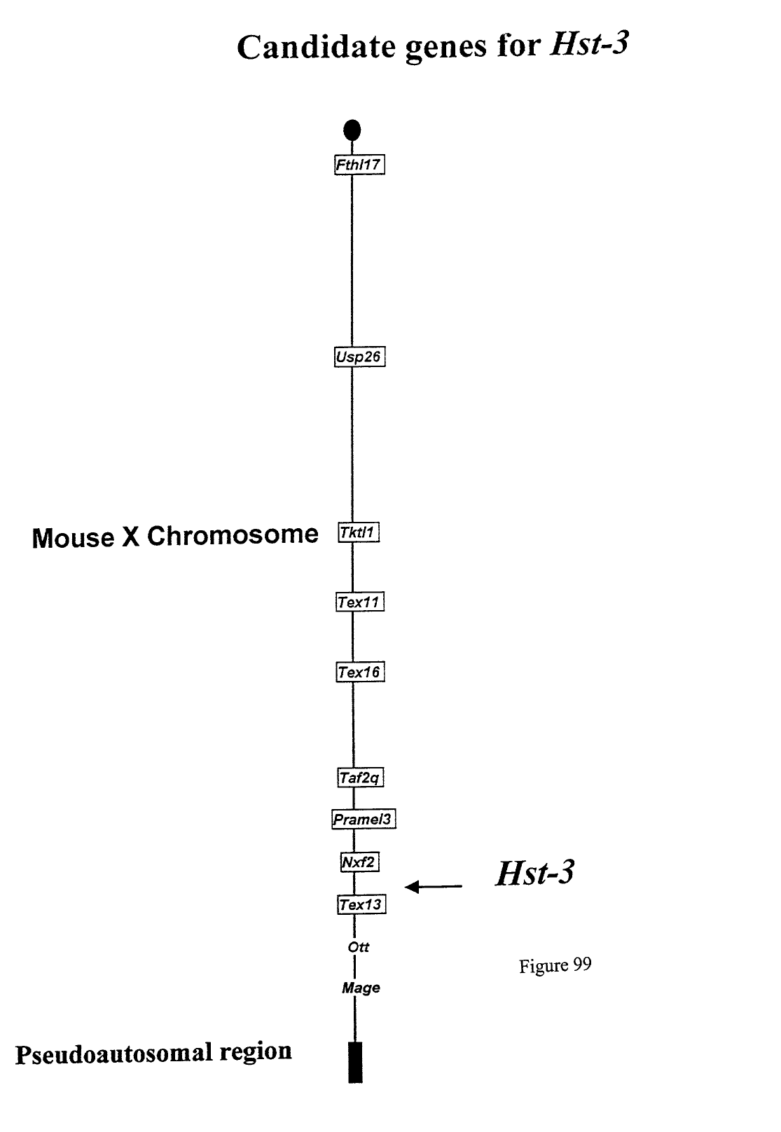

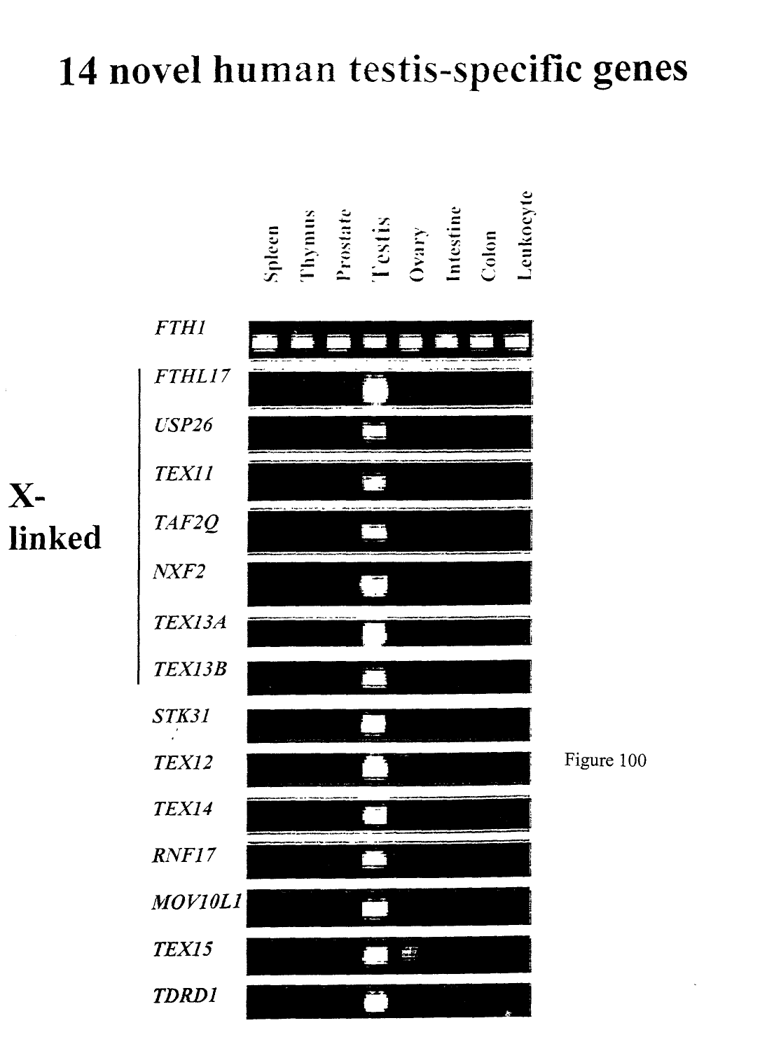

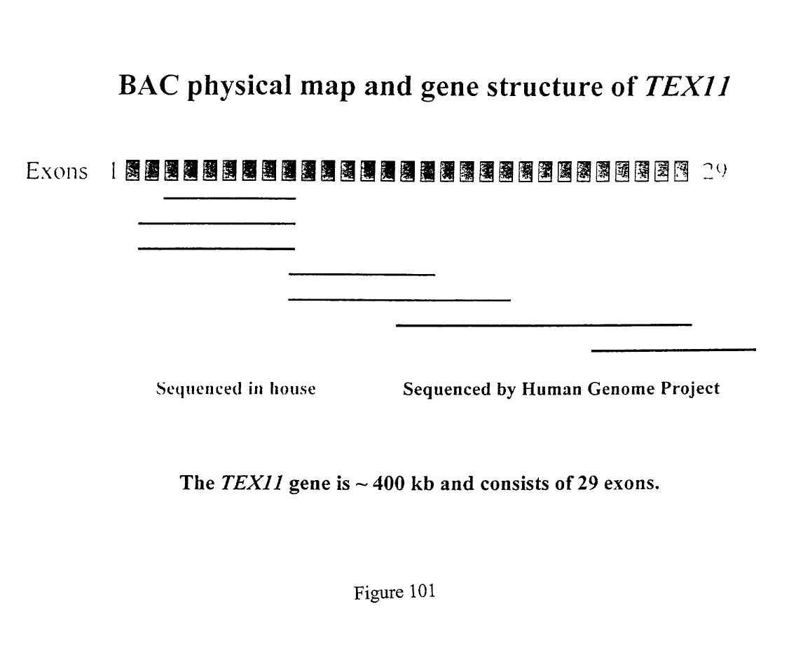

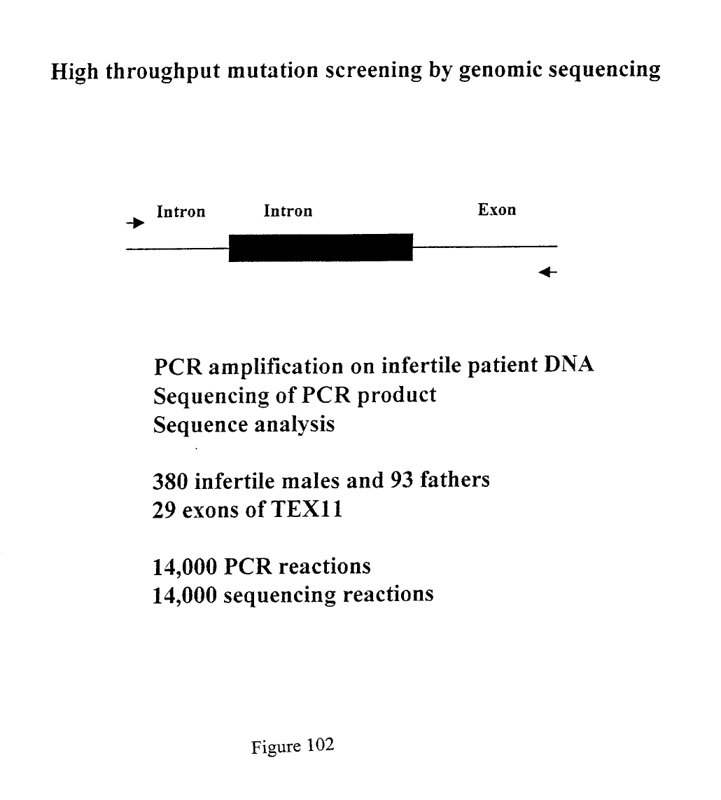

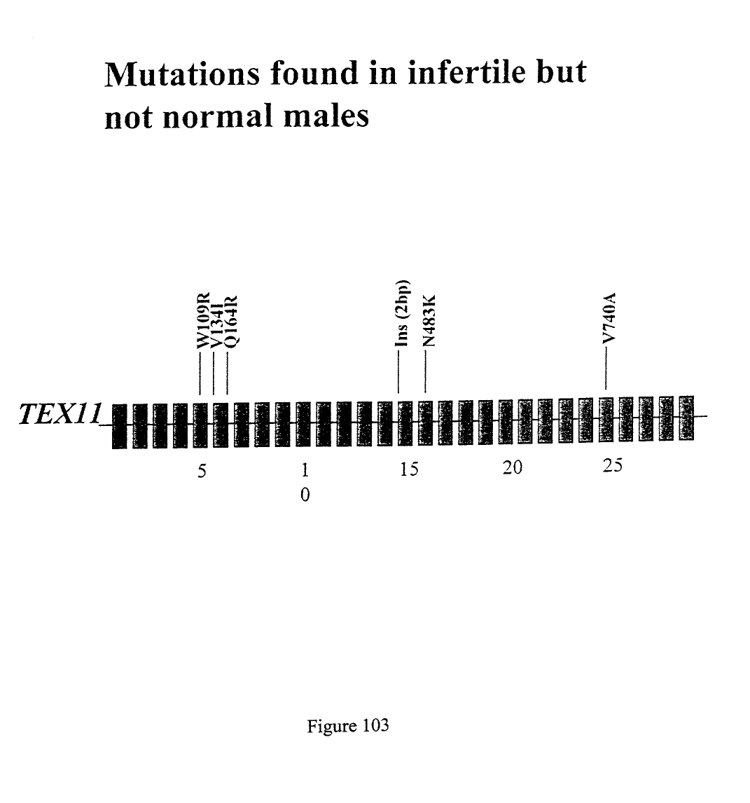

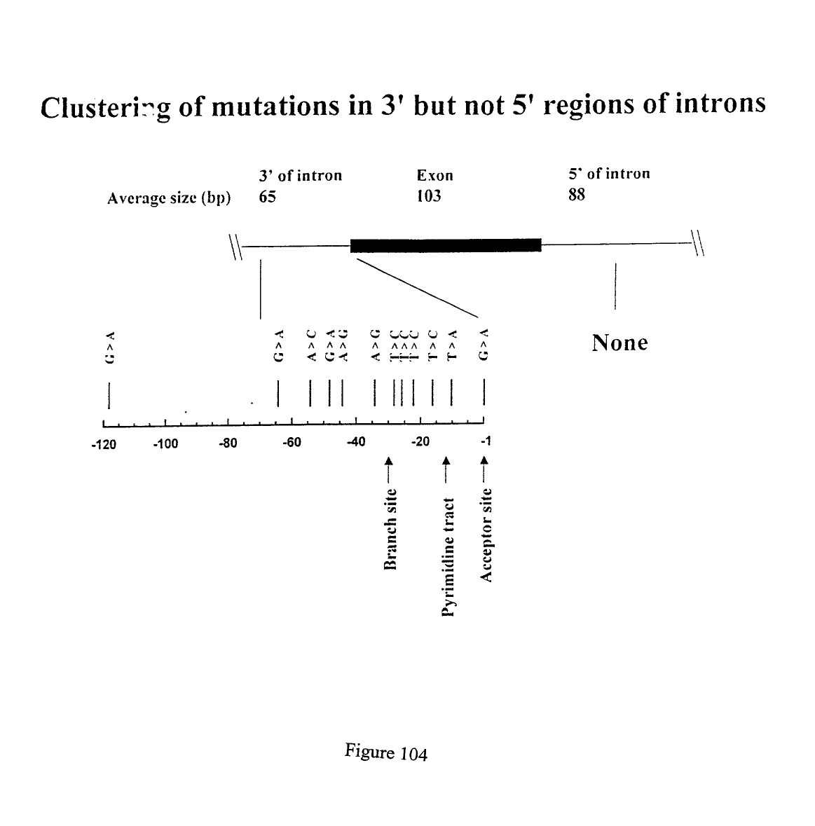

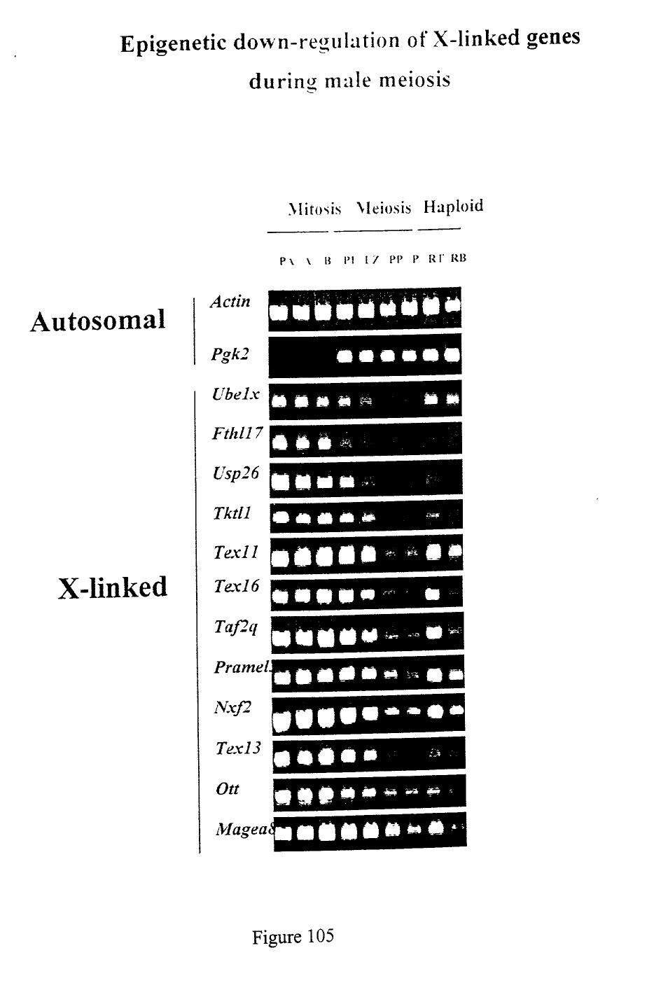

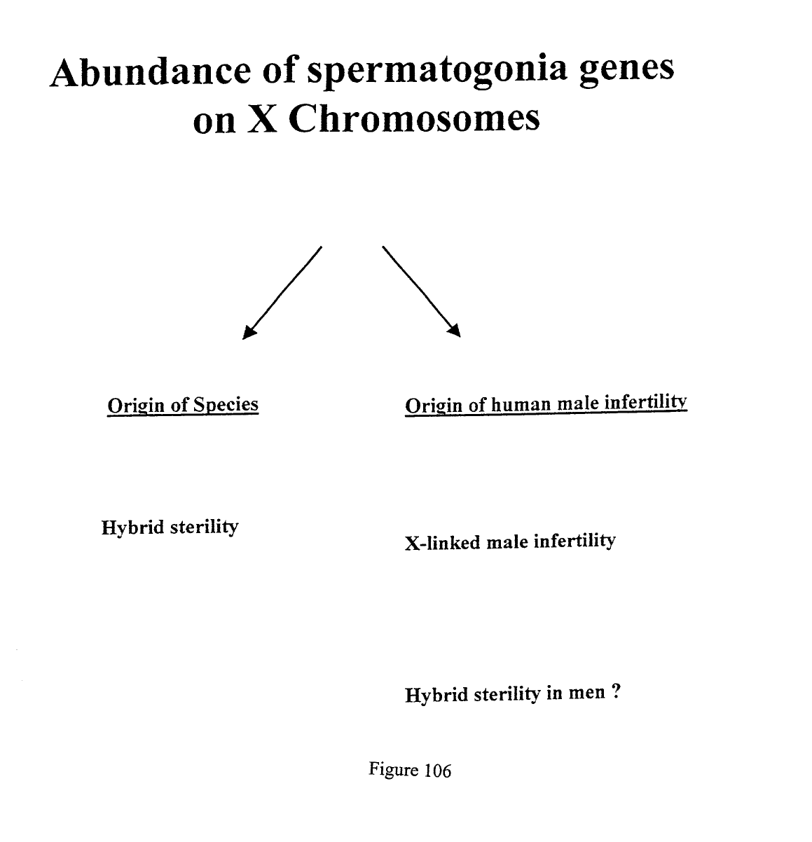

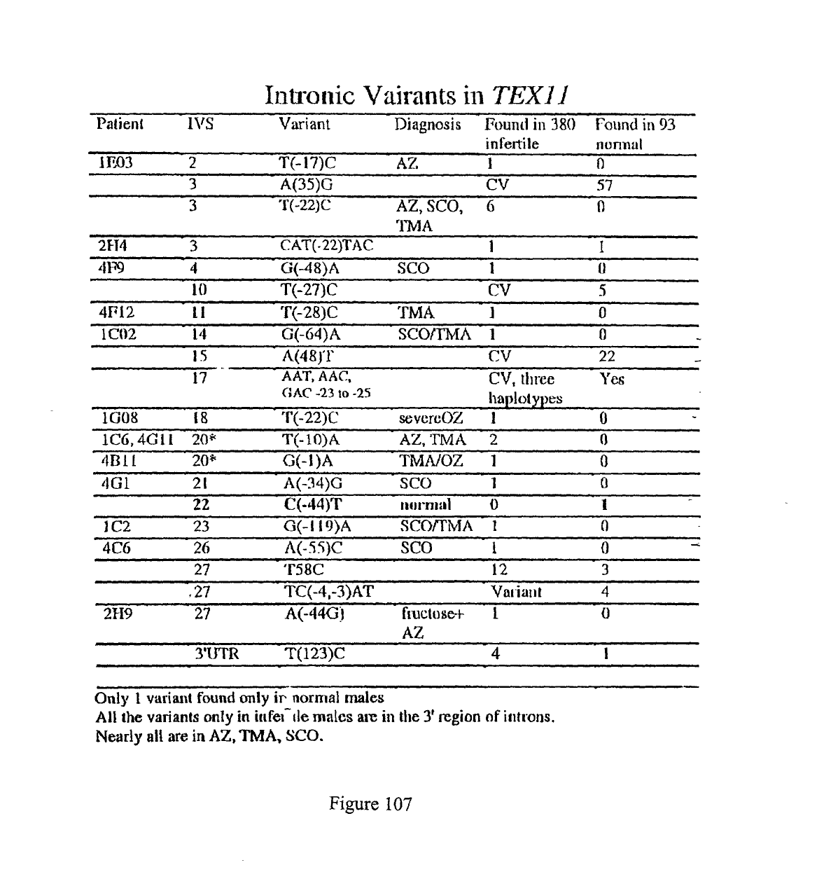

### DETAILED DESCRIPTION OF THE INVENTION

A description of preferred embodiments of the invention follows.

Described herein are isolated reproduction-specific nucleic acid molecules which are male germ cell-specific, testis-specific or testis- and ovary-specific. Also described are portions of the reproduction-specific nucleic acid molecules; complements of the reproduction-specific nucleic acid molecules and portions thereof and; nucleic acid molecules which hybridize to any of the reproduction-specific nucleic acid molecules under conditions of high stringency. Also described are nucleic acid molecules which are at least 70% identical in sequence to a reproduction-specific nucleic acid molecule whose sequence is presented herein or to a nucleic acid molecule which encodes a reproduction-specific protein whose amino acid sequence is presented herein, or to a nucleic acid molecule which hybridizes to any of the reproduction-specific nucleic acid molecules under conditions of high stringency.

Particularly preferred are nucleic acid molecules and portion thereof which have at least about 60%, preferably at least about 70, 80 or 85%, more preferably at least about 90%, even more preferably at least about 95%, and most preferably at least about 98% identity with nucleic acid molecules described herein.

In one embodiment, the nucleic acid molecules hybridize under high stringency hybridization conditions (e.g., for selective hybridization) to a nucleotide sequence described herein.

Stringent hybridization conditions for nucleic acid molecules are well known to those skilled in the art and can be found in standard texts such as Current Protocols in Molecular Biology, John Wiley & Sons, N.Y. (1998), pp. 2.10.1-2.10.16 and 6.3.1-6.3.6, the teachings of which are hereby incorporated by reference. As understood by those of ordinary skill, the exact conditions can be determined empirically and depend on ionic strength, temperature and the concentration of destabilizing agents such as formamide or denaturing agents such as SDS. Other factors considered in determining the desired hybridization conditions include the length of the nucleic acid sequences, base composition, percent mismatch between the hybridizing sequences and the frequency of occurrence of subsets of the sequences within other non-identical sequences. In one non-limiting example, nucleic acid molecules are allowed to hybridize in 6×sodium chloride/sodium citrate (SSC) at about 45° C., followed by one or more low stringency washes in 0.2×SSC/0.1% SDS at room temperature, or by one or more moderate stringency washes in 0.2×SSC/0.1% SDS at 42° C., or washed in 0.2×SSC/0.1% SDS at 65° C. for high stringency. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, preferably at least 40%, more preferably at least 60%, and even more preferably at least 70%, 80% or 90% of the length of the reference sequence. The actual comparison of the two sequences can be accomplished by well-known methods, for example, using a mathematical algorithm. A non-limiting example of such a mathematical algorithm is described in Karlin et al., Proc. Natl. Acad. Sci. USA, 90:5873-5877 (1993). Such an algorithm is incorporated into the NBLAST and XBLAST programs (version 2.0) as described in Altschul et al., Nucleic Acids Res., 25:389-3402 (1997). When utilizing BLAST and Gapped BLAST programs, the default parameters of the respective programs (e.g., NBLAST) can be used. See http://www.ncbi.nlm.nih.gov. In one embodiment, parameters for sequence comparison can be set at score=100, wordlength=12, or can be varied (e.g., W=5 or W=20).

A mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) PNAS, 85:2444-8.

The percent identity between two amino acid sequences can be accomplished using the GAP program in the CGC software package (available at http://www.cgc.com) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the CGC software package (available at http://www.cgc.com), using a gap weight of 50 and a length weight of 3. Thus, a substantially homologous amino acid or nucleotide sequence means an amino acid or nucleotide sequence that is largely but not wholly homologous to a nucleic acid molecule described herein, and which retains the same functional activity as the molecule to which it is homologous.

Also described herein are variant reproduction-specific nucleic acid molecules which are characteristic/indicative of infertility in men; mRNAs from which the cDNA is transcribed (mRNAs that encode the cDNA); proteins encoded by each of the nucleic acid molecules presented herein and by variations thereof (nucleic acid molecules that, due to the degeneracy of the genetic code, encode an amino acid sequence presented herein or a functional equivalent thereof); variant proteins associated with or indicative of lack of or reduction in sperm count (variant reproduction-specific proteins); characteristic portions of each of the proteins described herein; and antibodies that bind reproduction-specific proteins or variant reproduction-specific proteins or characteristic portions of these proteins.

The SEQ ID NO. for each of the sequences presented herein is shown in Table 1. Where shown, lower case letters in the figures indicate untranslated regions of the DNA. However, not all untranslated regions are shown in lower case letters. The skilled artisan can determine the appropriate coding region for each cDNA described herein using methods (e.g., computer programs) that are routine in the art.

TABLE 1
List of Sequence ID Numbers for cDNA, Protein and Genomic Sequences

| SEQ ID | | | | |
|---|---|---|---|---|
| NO. | Gene Name | Gene Symbol | Sequence | GenBank-NO. |
| 1 | Spg1 | Taf2q | cDNA | AF285574 |
| 2 | Spg1 | Taf2q | Protein | AF285574 |
| 3 | Spg2 | Tex11 | cDNA | AF285572 |
| 4 | Spg2 | Tex11 | Protein | AF285572 |
| 5 | Spg3 | Nxf2 | cDNA | AF285575 |
| 6 | Spg3 | Nxf2 | Protein | AF285575 |
| 7 | Spg5 | Tex15 | cDNA | AF285589 |
| 8 | Spg5 | Tex15 | Protein | AF285589 |
| 9 | Spg13 | Rnfl7 | cDNA | AF285585 |
| 10 | Spg13 | Rnfl7 | Protein | AF255585 |
| 11 | Spg14 | Scmh2 | cDNA | AF285577 |
| 12 | Spg14 | Scmh2 | Protein | AF285577 |
| 13 | Spg15 | Mov1011 | cDNA | AF285587 |
| 14 | Spg15 | Mov1011 | Protein | AF285587 |
| 15 | Spg16 | Piwil2 | cDNA | AF285586 |
| 16 | Spg16 | Piwil2 | Protein | AF285586 |
| 17 | Spg17 | Tktl1 | cDNA | AF285571 |
| 18 | Spg17 | Tktl1 | Protein | AF285571 |
| 19 | Spg18 | Tex12 | cDNA | AF285582 |
| 20 | Spg18 | Tex12 | Protein | AF285582 |
| 21 | Spg25 | Usp26 | cDNA | AF285570 |
| 22 | Spg25 | Usp26 | Protein | AF285570 |
| 23 | Spg27 | | cDNA | |
| 24 | Spg27 | | Protein | |
| 25 | Spg33 | Tex19 | cDNA | AF285590 |
| 26 | Spg33 | Tex19 | Protein | AF285590 |
| 27 | Spg34 | Fthl17 | cDNA | AF285569 |
| 28 | Spg34 | Fthl17 | Protein | AF285569 |
| 29 | Spg39 | Tex13 | cDNA | AF285576 |
| 30 | Spg39 | Tex13 | Protein | AF285576 |
| 31 | Spg46 | Stk31 | cDNA | AF255580 |
| 32 | Spg46 | Stk31 | Protein | AF285580 |
| 33 | Spg58 | Tex16 | cDNA | AF285573 |
| 34 | Spg58 | Tex16 | Protein | AF285573 |
| 35 | Spg59 | Tex20 | cDNA | AF285588 |
| 36 | Spg59 | Tex20 | Protein | AF285588 |
| 37 | Spg64 | | cDNA | |
| 38 | Spg64 | | Protein | |
| 39 | Spg65 | Rnh2 | cDNA | AF285581 |
| 40 | Spg65 | Rnh2 | Protein | AF285581 |
| 41 | Spg69 | Pramel1 | cDNA | AF285578 |
| 42 | Spg69 | Pramel1 | Protein | AF285578 |
| 43 | Spg70 | Tdrd1 | cDNA | AF285591 |
| 44 | Spg70 | Tdrd1 | Protein | AF285591 |
| 45 | Spg85 | Tex14 | cDNA | AF285584 |
| 46 | Spg85 | Tex14 | Protein | AF285584 |
| 47 | Spg87 | Tex18 | cDNA | AF285583 |
| 48 | Spg87 | Tex18 | Protein | AF285583 |
| 49 | Spg84 | Tex17 | cDNA | AF285579 |
| 50 | hSPG1 | TAF2Q | cDNA | AF285595 |
| 51 | hSPG1 | TAF2Q | Protein | AF285595 |
| 52 | hSPG3a | NXF2 | cDNA | AF285596 |
| 53 | hSPG3a | NXF2 | Protein | AF285596 |
| 54 | hSPG3a | | Genomic | |
| 55 | hSPG3b | | cDNA | |
| 56 | hSPG5 | TEX15 | cDNA | AF285605 |
| 57 | hSPG5 | TEX15 | Protein | AF285605 |
| 58 | hSPG5 | | Genomic | |
| 59 | hSPG15 | MOV10L1 | cDNA | AF285604 |
| 60 | hSPG15 | MOV10L1 | Protein | AF285604 |
| 61 | hSPG15 | | Genomic | |
| 62 | hSPG18 | TEX12 | cDNA | AF285600 |
| 63 | hSPG18 | TEX12 | Protein | AF285600 |
| 64 | hSPG25 | USP26 | cDNA | AF285593 |
| 65 | hSPG25 | USP26 | Protein | AF285593 |
| 66 | hSPG27 | | cDNA | |
| 67 | hSPG34a | | cDNA | |
| 68 | hSPG34a | | Protein | |
| 69 | hSPG34b | FTHL17 | cDNA | AF285592 |
| 70 | hSPG34b | FTHL17 | Protein | AF285592 |
| 71 | hSPG39a | TEX13A | cDNA | AF285597 |
| 72 | hSPG39a | TEX13A | Protein | AF285597 |
| 73 | hSPG39a | | Genomic | |
| 74 | hSPG39b | TEX13B | cDNA | AF285598 |
| 75 | hSPG46 | STK31 | cDNA | AF285599 |
| 76 | hSPG46 | STK31 | Protein | AF285599 |
| 77 | hSPG64 | | cDNA | |
| 78 | hSPG64 | | Protein | |
| 79 | hSPG85 | TEX14 | cDNA | AF285601 |
| 80 | hSPG85 | TEX14 | Protein | AF285601 |
| 81 | hSPG13 | RNF17 | cDNA | AF285602 |
| | long | | | |
| 82 | hSPG13 | RNF17 | Protein | AF285602 |
| | long | | | |
| 83 | hSPG13 | RNF17 | cDNA | AF285603 |
| | short | | | |
| 84 | hSPG13 | RNF17 | Protein | AF285603 |
| | short | | | |
| 85 | hSPG39b | TEX13B | Protein | AF285598 |
| 86 | hSPG39b | | Genomic | |
| 87 | hSPG70 | TDRD1 | cDNA | AF285606 |
| 88 | hSPG70 | TDRD1 | Protein | AF285606 |
| 89 | hSPG2 | TEX11 | cDNA | AF285594 |
| 90 | hSPG2 | TEX11 | Protein | AF285594 |

As used herein, the terms “reproduction-specific nucleic acid molecules” and “reproduction-specific genes” refer, respectively, to reproduction-specific nucleic acid molecules and reproduction-specific genes which are male germ cell-specific, testis-specific or testis- and ovary-specific. As used herein, the terms “variant reproduction-specific nucleic acid molecules” and “variant reproduction-specific genes” refer, respectively, to reproduction-specific nucleic acid molecules and reproduction-specific genes which are male germ cell-specific, testis-specific or testis- and ovary-specific. Variant reproduction-specific nucleic acid molecules or genes can differ from reproduction-specific nucleic acid molecules in nucleic acid sequence (e.g., deletion of one or more nucleotides, addition of one or more nucleotides or substitution or change in one or more nucleotides) or by their “loss” either physically or by failure of/or reduction in expression.

As used herein, the term “isolated” refers to substances which are obtained from (separated from) the sources in which they occur in nature, as well as to substances (e.g., nucleic acid molecules, proteins, peptides) produced by recombinant/genetic engineering methods or by synthetic (chemical) methods.

Also the subject of the present invention are methods in which the nucleic acid molecules, proteins, and antibodies of the present invention are useful. Such methods include a method of identifying genes or proteins characteristic of male infertility, which include variant genes or proteins present in infertile men, but not in fertile men, and nucleic acid molecules or proteins present at different levels or at a different stage(s) in differentiation in infertile men than in fertile men. Also included is a method of diagnosing or aiding in the diagnosis of infertility in men, and a method of contraception in which sperm production or sperm count is reduced (no sperm is produced, sperm is produced to a lesser extent than normal in an individual) or defective sperm is produced (e.g., sperm with reduced motility, lifespan or testicular maturation arrest, or sertoic cell defects ). As used herein, the terms “infertility in men” or “male infertility” include spermatogenic failure, a lack of sperm production, a severely reduced sperm count and production of defective sperm, each of which results in the inability or a severely reduced ability to cause fertilization.

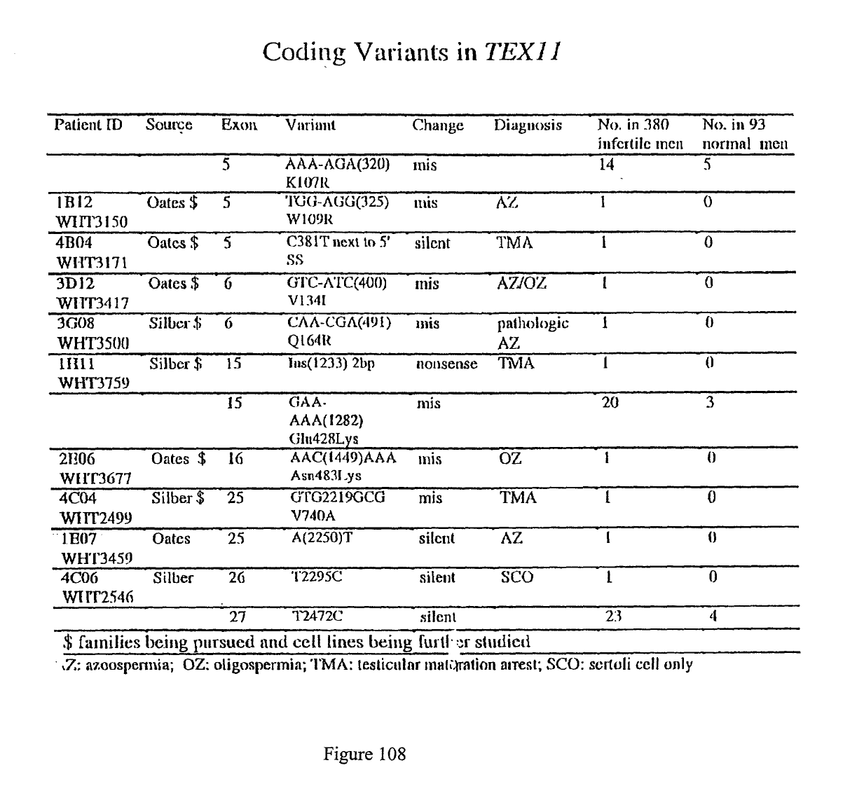

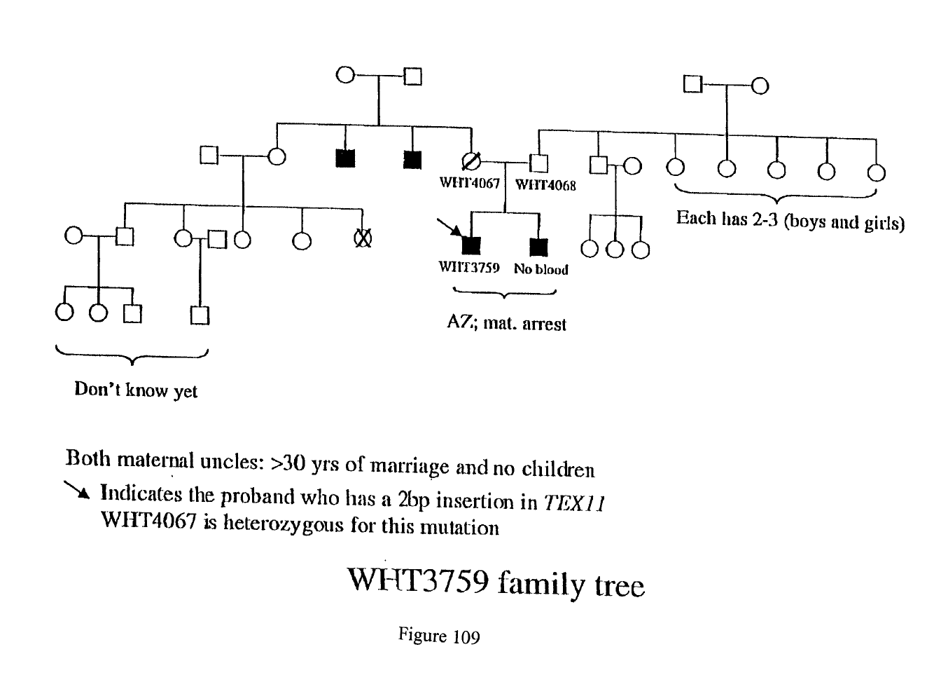

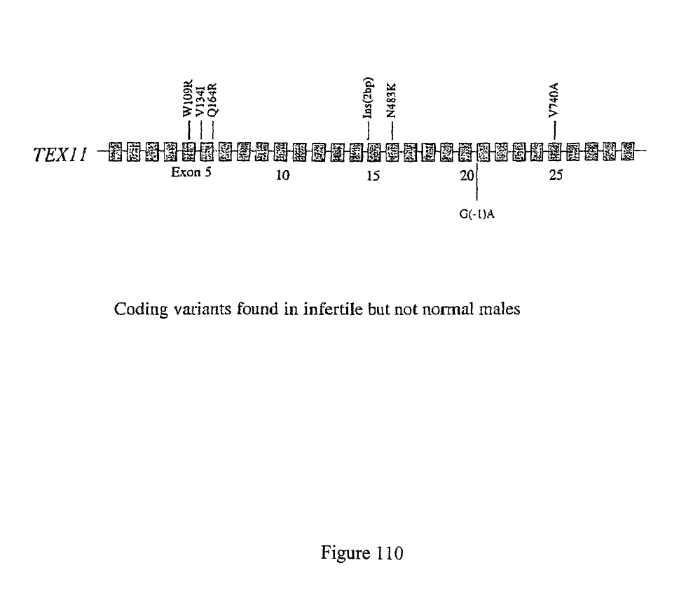

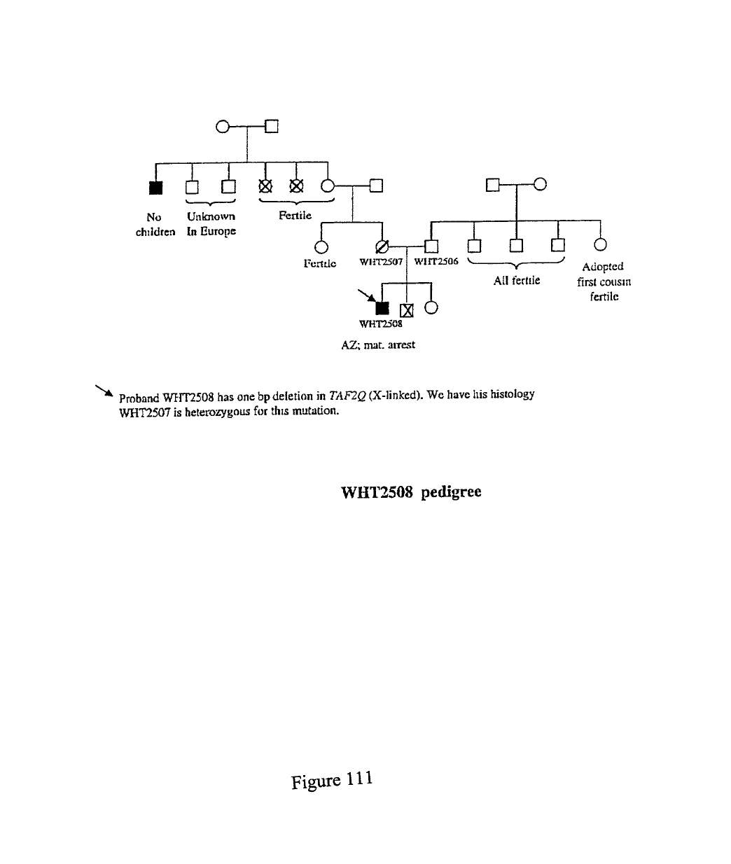

Tex11 is a reproduction-specific gene that is X chromosome-linked. Its 3 kb cDNA encodes a 917-residue protein that has no homology with other known proteins. The Tex11 gene is approximatly 400 kb and consists of 29 exons. As described in Example 2, 380 infertile males and 93 fertile males (fathers) were studied and 33 mutations were found in the nucleic acid sequence of TEX11; of these, 21 were found only in infertile males. These mutations include A320G, T325A, C381T, G400A, A491G, G1282A, C1449A, T2219C, A2250T, T2295C and T2472C and also shown is a two base pair insertion in exon 15 at nucleotide position 1233 (denoted as ins(2 bp)) in FIG. 108. A clustering of mutations is found in the 3′ but not the 5′ regions of the intron. These nucleic acid alterations are shown in FIG. 108.

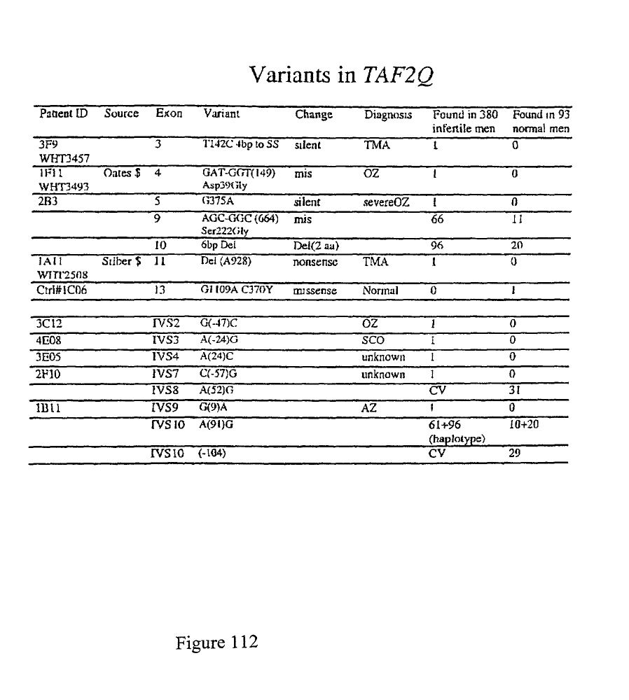

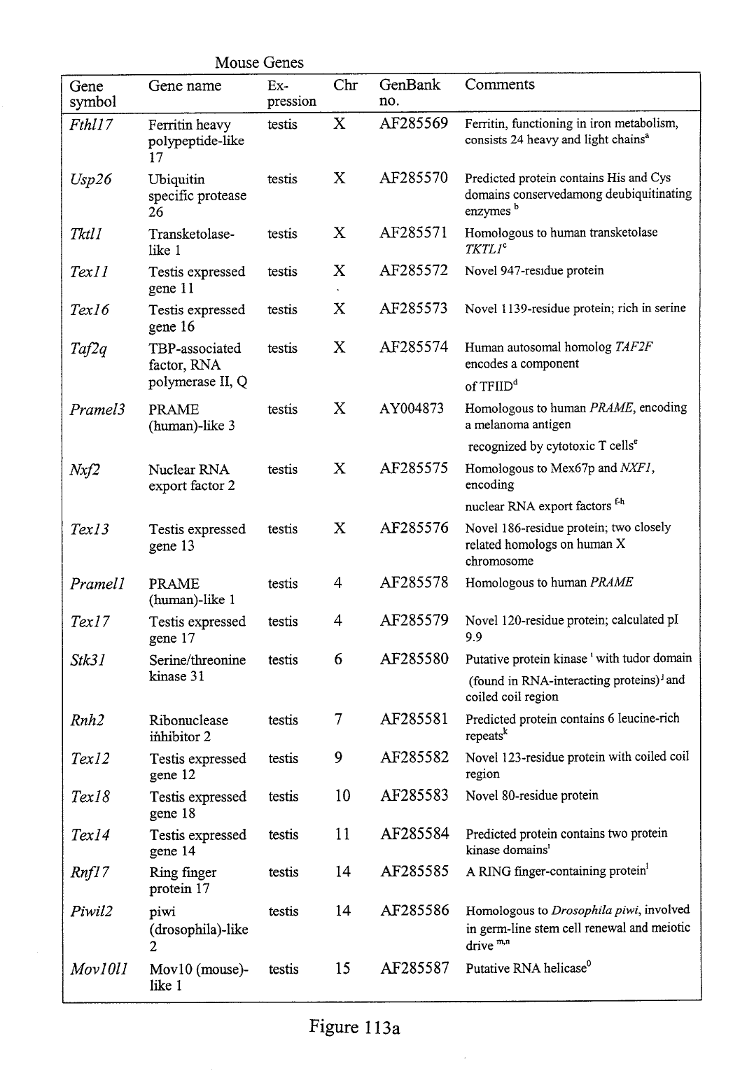

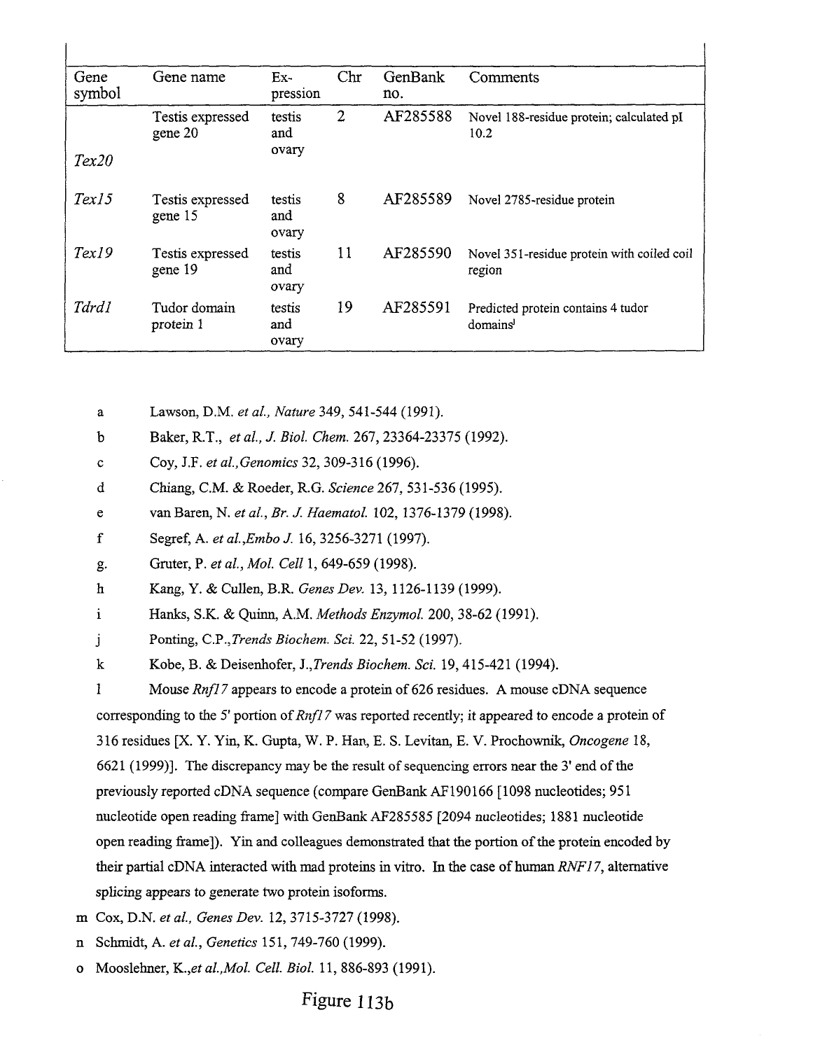

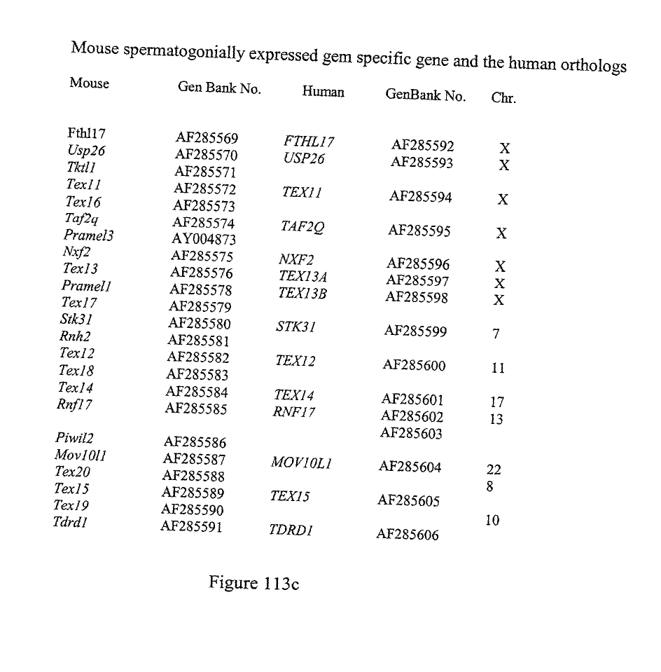

Another X linked reproduction-specific gene identified as containing variants as described herein is TAF2Q. The TAF2Q DNA and amino acid variations associated with infertility are shown in FIG. 112.

Isolated nucleic acid molecules (nucleic acid molecule genes, cDNAs, mRNA, RNA) of the present invention are of mammalian origin, such as of mouse (designated as Spg), human (designated as hspg) or other primate, canine, feline or bovine origin.

Both reproduction-specific nucleic acid molecules and variant reproduction-specific nucleic acid molecules are useful as hybridization probes or primers for an amplification method, such as polymerase chain reaction, to show the presence, absence or alteration of a gene(s) described herein. Probes and primers can comprise all or a portion of the nucleotide sequence (nucleic acid sequence) of a reproduction-specific nucleic acid molecule described herein or all or a portion of its complement. They can also comprise all or a portion of a variant reproduction-specific nucleic acid molecule which portion is characteristic of (indicative of) infertility or all or a portion of its complement. The probes and primers can be of any length, provided that they are of sufficient length and appropriate composition (appropriate nucleotide sequence) to hybridize to all or an identifying or characteristic portion of a gene indicative of infertility in men and remain hybridized under the conditions used. Useful probes include nucleic acid molecules which distinguish between a reproduction-specific nucleic acid molecule described herein and a variant form of such a nucleic acid molecule that is indicative of infertility in men. Generally, the probe will be at least 14 nucleotides; the upper limit is the length of the nucleic acid molecule itself. Probes can be, for example, 14 to 20 nucleotides or longer (e.g., 15 to 25, 20 to 40, 30 to 50 or any other length appropriate to specifically hybridize to a reproduction-specific gene or a variant reproduction-specific nucleic acid molecule and remain hybridized to nucleic acid molecules in a sample under the conditions used). The length of a specific probe will also be determined by the method in which it is used.

The genes described herein are useful to detect variant reproduction-specific nucleic acid molecules present in a nucleic acid molecule sample obtained from men with lack of or reduction in sperm production, but not present in a nucleic acid molecule sample obtained from fertile men. Variant reproduction-specific nucleic acid molecules (e.g., having large alterations or deletions and small alterations or deletions such as short deletions, point mutations and small insertions) can be identified with reference to reproduction-specific nucleic acid molecules/gene sequences presented herein. For example, nucleic acid molecules from infertile men with normal karyotypes and no Y chromosome microdeletions can be assessed. All human spermatogonic genes can be screened in a group of infertile men (with no or low sperm counts) using PCR. One pair of PCR primers can be designed for each spermatogonic gene to produce a 200 bp PCR product or a PCR product of any appropriate length. A negative PCR result indicates the absence of a particular gene in an individual and can be confirmed by Southern blot. Small variations can be searched for in X-linked genes by nucleic acid molecule sequencing. Fertile men are used as controls. If a variant reproduction-specific gene is identified, additional infertile men can be similarly screened to further confirm that the variant reproduction-specific nucleic acid molecule is associated with/indicative of infertility in men. Alterations which are specific to infertile men can be used in the diagnosis of male infertility, alone or in conjunction with other methods of assessing male infertility.

The spermatogonic genes are strong candidates for pure male sterility factors. A mutation in such a gene could alter its function in spermatogenesis and therefore cause male infertility. These novel genes are promising for the following reasons: first, they are germ cell-specific and expressed in spermatogonia. Two known germ cell-specific Y-linked human genes, RBM and DAZ, are also expressed in spermatogonia and are strongly implicated in male infertility when deleted. The mouse homologues of RBM and DAZ were also identified in the subtraction protocol described in the Examples, suggesting an important role for other spermatogonic genes in male fertility. Second, nearly 50% of novel germ cell-specific genes are located on Chromosome X. This is significant from a theoretical point of view, indicating that Chromosome X may play the most important role in male fertility. From a practical point of view, this result shows that mutations in infertile men are more likely to be found in X-linked genes than in autosomal genes. It is also far easier to search the X chromosome than within autosomes. In males, there is only one copy of the X-linked gene. For example, to find a mutation with a frequency of 1% in the population, one can screen 100 individuals if it is X-linked. If the gene is autosomal, one has to screen 10,000 individuals (1%×1%=0.01%) to find a homozygous mutation. However, the method described herein applies to the search for variations in infertile men in both X-linked and autosomal genes of this invention.

In a further embodiment, the present invention is a method of diagnosing reduced (partially or totally) sperm count or infertility in a man. For example, a method of diagnosing infertility in a man comprises (a) comparing the nucleic acid sequence of reproduction-specific nucleic acid molecules obtained from a man in whom infertility is to be assessed with the nucleic acid sequence of a corresponding variant reproduction-specific nucleic acid molecules from infertile men, wherein the corresponding variant reproduction-specific nucleic acid molecules comprises an alteration characteristic of infertility in men; and (b) determining whether the alteration characteristic of infertility in men is present in the reproduction-specific nucleic acid molecules obtained from the man in whom fertility is to be assessed. If the alteration is present in the nucleic acid molecules obtained, infertility is diagnosed in the man. A corresponding variant reproduction-specific nucleic acid molecule is a reproduction-specific nucleic acid molecule of the same chromosomal location as the chromosomal location of nucleic acid molecule being analyzed (a nucleic acid molecule obtained from a man being assessed). One or more of the nucleic acid molecules described herein, or a portion(s) of one or more of the nucleic acid molecules or nucleic acid molecules that hybridize to nucleic acid molecules described herein or to a complement thereof can be used in a diagnostic method, such as a method to determine whether a gene(s) or a portion of a gene(s) described herein is missing or altered in men. Any man may be assessed with this method of diagnosis. In general, the man will have been at least preliminarily assessed, by another method, as having reduced sperm count. By combining nucleic acid probes derived from a sequence presented herein that is present in the DNA of fertile men, but not in the DNA of infertile men, with the nucleic acid molecules from a sample to be assessed, under conditions suitable for hybridization of the probes with DNA present in fertile men, but not with variant DNA, it can be determined whether the sample from a man to be assessed comprises the variant reproduction-specific nucleic acid molecules. If the nucleic acid molecule is unaltered (is not a variant reproduction-specific nucleic acid molecules), it may be concluded that the alteration of the gene is not responsible for the reduced sperm count. Alternatively, the hybridization conditions used can be such that the probes will hybridize only with variant reproduction-specific nucleic acid molecules and not with reproduction-specific nucleic acid molecules.

Nucleic acid molecules assessed by the present method can be obtained from a variety of tissues and body fluids, such as blood or semen. In one embodiment, the above methods are carried out on nucleic acid molecules obtained from a blood sample. For example, a nucleic acid sample from men who are infertile or have a low sperm count is assessed to determine whether all or a portion of a nucleic acid molecule(s) described herein differs in sequence from the sequence of a corresponding nucleic acid molecule obtained from fertile men. In one embodiment, the altered nucleic acid molecules or gene which is assessed is one which differs from a sequence described herein by a deletion, addition or substitution of at least one nucleotide. In a second embodiment, the altered nucleic acid molecule or gene is “missing” in that it is physically absent or not expressed/under-expressed (functionally absent). If an alteration occurs in a nucleic acid molecule obtained from infertile men, but not fertile men, it is indicative of (characteristic of) infertility and, thus, useful in the diagnosis of infertility in men. Such a nucleic acid molecule or gene is referred to as variant reproduction-specific nucleic acid molecule or variant reproduction-specific gene.

This invention also relates to proteins encoded by the genes or portions of the genes described herein, proteins encoded by variant nucleic acid molecules (or portions thereof) that are characteristic of infertility in men), or by portions thereof and antibodies that recognize (bind) proteins described herein. Such antibodies are useful in a diagnostic method to determine whether an intact or variant protein(s) is present in a sample (e.g., semen or testis biopsy) obtained from a man being assessed for infertility. They are also useful for identifying the expression of the gene(s) in a particular cell type or at a particular developmental stage. These antibodies can be used for studies of spermatogenesis. These antibodies can be used for immunofluorescence of germ cells, or in Western blots for assessing the presence of the protein the antibody binds.

The invention also provides expression vectors containing a reproduction-specific nucleic acid molecule of the present invention which is operably linked to at least one regulatory sequence. “Operably linked” is intended to mean that the nucleotide sequence is linked to a regulatory sequence in a manner which allows expression of the nucleotide sequence. The term “regulatory sequence” includes promoters, enhancers, and other expression control elements (see, e.g., Goeddel, Gene Expression Technology: Methods in Enzymology 185, Academic Press, San Diego, Calif. (1990)). It should be understood that the design of the expression vector may depend on such factors as the choice of the host cell to be transformed and/or the protein or peptide desired to be expressed. For instance, the proteins and peptides of the present invention can be produced by ligating the cloned gene, or a portion thereof, into a vector suitable for expression in either prokaryotic cells, eukaryotic cells or both (see, for example, Broach, et al., Experimental Manipulation of Gene Expression, ed. M. Inouye (Academic Press, 1993) p. 83; Molecular Cloning: A Laboratory Manual, 2^{nd} Ed., Sambrook et al. (Cold Spring Harbor Laboratory Press, (1989) Chapters 16 and 17).

Prokaryotic and eukaryotic host cells transfected by the described vectors are also provided by this invention. For instance, cells which can be transfected with the vectors of the present invention include, but are not limited to, bacterial cells, such as E. coli, insect cells (baculovirus), yeast and mammalian cells, such as Chinese hamster ovary (CHO) cells.

Thus, a nucleotide sequence described herein can be used to produce a recombinant form of the encoded protein via microbial or eukaryotic cellular processes. Production of a recombinant form of the protein can be carried out using known techniques, such as by ligating the oligonucleotide sequence into a DNA or RNA construct, such as an expression vector, and transforming or transfecting the construct into host cells, either eukaryotic (yeast, avian, insect or mammalian) or prokaryotic (bacterial cells). Similar procedures, or modifications thereof, can be employed to prepare recombinant proteins according to the present invention by microbial means or tissue-culture technology.

The present invention also pertains to pharmaceutical compositions comprising the proteins and peptides described herein. For instance, the peptides or proteins of the present invention can be formulated with a physiologically acceptable medium to prepare a pharmaceutical composition. The particular physiological medium may include, but is not limited to, water, buffered saline, ployols (e.g., glycerol, propylene glycol, liquid polyethylene glycol) and dextrose solutions. The optimum concentration of the active ingredient(s) in the chosen medium can be determined empirically, according to procedures well known in the art, and will depend on the ultimate pharmaceutical formulation desired. Methods of introduction of exogenous polypeptides at the site of treatment include, but are not limited to, intradermal, intramuscular, intraperitoneal, intravenous, subcutaneous, oral and intranasal methods. Other suitable methods of introduction can also include rechargeable or biodegradable devices and slow release polymeric devices. The pharmaceutical compositions of this invention can also be administered as part of a combinatorial therapy with other agents.

This invention also has utility in methods of treating disorders of reduced sperm count or enhancing/increasing sperm count and/or sperm activity. Reduced sperm count can be increased, for example, by administering a drug or agent that enhances the activity of a reproduction-specific gene or genes, with the result that sperm count is enhanced. Alternatively it can be used in a method of gene therapy, whereby the gene or a gene portion encoding a functional protein is inserted into cells in which the functional protein is expressed and from which it is generally secreted to remedy the deficiency caused by the defect in the native gene.

The invention described herein also has application to the area of male contraceptives. Variant reproduction-specific genes indicative of infertility can be used to design agents which mimic the activity of the altered gene product(s). Thus, the present invention also relates to agents or drugs, such as, but not limited to, peptides or small organic molecules which mimic the activity (effects) of the variant gene product(s) of reproduction-specific genes (a variant reproduction-specific protein) of the present invention shown to be present in infertile men, but not in fertile men. One embodiment of this invention is a method of contraception (a method of reducing sperm production and/or sperm activity) in a man, comprising administering to the man an agent that mimics the effects of a variant reproduction-specific protein in the man, whereby sperm production, sperm activity or both are reduced (and preferably abolished) in the man.

Alternatively, the agent or drug is one which blocks or inhibits the expression, activity or function of the reproduction-specific gene (e.g., an oligonucleotide or a peptide which blocks or inhibits the expression, activity or function of a reproduction-specific gene present in nucleic acid molecules of fertile men). The ideal agent will enter the cell, in which it will block or inhibit the function of the gene, directly or indirectly. Alternatively, an agent or drug can inhibit the activity or function of one or more proteins encoded by reproduction-specific nucleic acid molecules.

Reproduction-specific nucleic acid molecules described herein, such as those that encode proteins which have enzymatic activity, are potential targets of such blocking agents or inhibitors, as are the encoded proteins. For example, Spg17, which encodes a transketolase, and its human homologue; Spg25, which encodes a deubiquitinating enzyme, and its human homologue enzyme; Spg65, which encodes a RNase inhibitor, and its human homologue; and Spg85, which encodes a tyrosine protein kinase, and its human homologue can be targets of inhibitors, as can the encoded proteins. Agents that inhibit the gene, directly or indirectly, and/or the encoded product, directly or indirectly, are potential contraceptive agents. Agents that inhibit the gene, directly or indirectly, and/or the encoded product, directly or indirectly, are potential contraceptive agents.

Identification of a blocking agent or inhibitor of a reproduction-specific gene or an encoded product can be carried out using known methods. For example, a gene for which an inhibitor is to be identified can be expressed in an appropriate host cell (e.g., mouse or human cell lines), in the presence of an agent or drug to be assessed for its ability to block or inhibit a reproduction-specific gene(s) (a candidate drug). The ability of the candidate drug to do so can be assessed in several ways. For example, its effect on expression of the gene (e.g., by determining if the gene product is present in the host cells, by immunoassay or Western blot) can be assessed. Alternatively, binding of the candidate drug to the reproduction-specific gene or to the encoded protein can be assessed, as can degradation or disruption of the gene or the encoded protein. For example, hSPG25 has two catalytic domains (Cys domain and His domain) that are conserved within the ubiquitin specific protease family (Usp) members. In a bacterial assay (Baker et al., J Biol Chem 267, 23364-75 (1992)), the enzyme encoded by hSPG25 might cleave the Ub (ubiquitin) moiety from the substrate Ub-Arg-β-Gal, a fusion protein of Ub and E. coli β galactosidase linked by an arginine. E. coli expressing Ub-Arg-β-gal only will form blue colonies in the presence of its chromogenic substrate X-Gal. A deubiquitinating enzyme, like hSPG25, introduced in E. coli would cleave Ub-Arg-β-Gal into Ub and Arg-β-Gal, which is an unstable protein, thus forming white colonies. A candidate drug would block the deubiquitinating activity of hSPG25. E. coli expressing both Ub-Arg-β-Gal and hSPG25 should form blue colonies in the presence of X-Gal and the candidate drug.

The present invention also relates to antibodies that bind a protein or peptide encoded by all or a portion of the reproduction-specific nucleic acid molecule, as well as antibodies which bind the protein or peptide encoded by all or a portion of a variant nucleic acid molecule. For instance, polyclonal and monoclonal antibodies which bind to the described polypeptide or protein are within the scope of the invention. In a specific embodiment, this invention relates to antibodies (polyclonal or monoclonal) that bind a protein or peptide that is associated with or indicative of infertility in men (a variant protein or peptide). Such antibodies can be used, alone or in combination with antibodies that bind proteins or peptides encoded by reproduction-specific nucleic acid molecules found in fertile men, in immunoassays carried out to diagnose or aid in the diagnosis of infertility.

Antibodies of this invention can be produced using known methods. An animal, such as a mouse, goat, chicken or rabbit, can be immunized with an immunogenic form of the protein or peptide (an antigenic fragment of the protein or peptide which is capable of eliciting an antibody response). Techniques for conferring immunogenicity on a protein or peptide include conjugation to carriers or other techniques well known in the art. The protein or peptide can be administered in the presence of an adjuvant. The progress of immunization can be monitored by detection of antibody titers in plasma or serum. Standard ELISA or other immunoassays can be used with immunogen as antigen to assess the levels of antibody. Following immunization, anti-peptide antisera can be obtained, and if desired, polyclonal antibodies can be isolated from the serum. Monoclonal antibodies can also be produced by standard techniques which are well known in the art (Kohler and Milstein, Nature 256:4595-497 (1975); Kozbar et al., Immunology Today 4:72 (1983); and Cole et al., Monoclonal Antibodies and Cancer Therapy, Alan R. Liss, Inc., pp. 77-96 (1985)). Such antibodies are useful as diagnostics for the intact or disrupted gene, and also as research tools for identifying either the intact or disrupted gene.

As described in Example 2, chromosomal mapping of the genes described herein demonstrated the surprisingly large number of genes on sex chromosome X. This is the strongest evidence to date in support of the population genetics theory first suggested by R. A. Fisher and formalized by W. Rice. (Fisher, R. A., Biol. Rev. 6, 345-368 (1931); Rice, W., Evolution 38, 735-742 (1996); Hurst, L. D. and J. P. Randerson, Trends Genet. 15, 383-385 (1999)). This theory argues that sexually antagonistic traits (beneficial in one sex, but detrimental or neutral in the other) on chromosome X tend to be strongly selected and, therefore, accumulate. Male germ cell-specific genes are only expressed in males and are, therefore, sexually antagonistic genes. The work described herein has resulted in identification of a number of testis-specific genes on chromosome X in both mice and humans.

In 1922, J B S Haldane observed that when in the offspring of two different animal races one sex is absent, rare, or sterile, that sex is the heterozygous sex (XY or ZW) (Haldane J B S., J. Genet. 12:101-109 (1922)). Thus, in humans, males (XY) are sterile and female (XX) are fertile. This rule is obeyed in all animals: lepidoptera, birds, flies and mammals. The significance of this is the early stage in speciation, known as the origin of species. Haldane's rule incorporates the following in his hypotheses: incompatibility between X- and Y linked genes, meiotic drive, disruption of dosage compensation, X-autosome translocation, dominance theory, faster-male theory and faster-X theory. The two assumptions made are that there are an abundance of “speciation genes” on X chromosome and the rapid evolution of “speciation genes”. The result of the male sterility is reproduction isolation and the origin of two species. Hybrid male sterility in mice has been mapped to Hst-1 and Hst-3 locus (Forejt J. et al., Mammalian Genome 1:84-91(1991); Matsuda Y. et al., Proc. Natl. Acad. Sci. USA 88:4850-4954 (1991)). In one study, the species M.m. musculus crossed with M.m. domesticus, the male sterility mapped to chromosome 17 t-complex (Hst-1 locus) and resulted in meiotic arrest of the spermatagonia. The X-Y dissociation and autosomal dissociation are high and the nature of the defect is genetic. In the other study, M. spretus crossed with M.m. domesticus resulting in male sterility mapped to chromosome X distal end producing meiotic arrest of the spermatagonia, The X-Y dissociation is high/low, the autosomal dissociation high/low and the mature of the defect may be structural.

The present invention is illustrated by the following Examples, which are not intended to be limiting in any way. The teaching of all references cited herein are incorporated by reference in their entirety.

### EXAMPLES

### Example 1

Isolation and Cloning of Reproduction-Specific Genes from Mice

Isolation of Mouse Spermatogonia

Spermatogonia were isolated by the Staput method of sedimentation velocity at unit gravity (Bellve, A. R.. Methods Enzymol. 225, 84-113 (1993)). Primitive type A spermatogonia were prepared from testes of 6-day-old CD-1 mice (Charles River Laboratories). Mature type A and type B spermatogonia were isolated from 8-day-old CD-1 mice. By microscopic examination, at least 85% of the cells in the resulting preparations were spermatogonia, with no more than 15% somatic cell contamination.

cDNA Subtraction

Three independent subtraction experiments were carried out using cDNAs from primitive type A, type A, or type B spermatogonia as the tracer. In all cases, tracer and driver cDNAs were derived from oligo(dT)-selected RNAs. Germ-cell-depleted testes were from w^{v}/w^{v} animals. Prior to subtraction, tracer and driver cDNAs were digested to completion with Rsa I. In each of the three experiments, we carried out one round of subtraction was performed using the “PCR-select” protocol (Clontech)(Diatchenko, L. et al. Proc. Natl. Acad. Sci. USA 93, 6025-6030 (1996). To more thoroughly subtract ubiquitous cDNAs, four additional rounds of subtraction were performed using a modified procedure (Douglas Menke, Whitehead Institute, personal communication) as described in Lavery, D. J.,et al., Proc. Natl. Acad. Sci. USA 94, 6831-6836 (1997). Between rounds of subtraction, enrichment of Dazl cDNA (germ-cell-specific) was monitored and disappearance of G3PDH cDNA (ubiquitous) was monitored. Three plasmid libraries (one for each of the three independent experiments) were prepared from the resulting pools of subtracted cDNA fragments. 800 randomly selected clones from each of the three libraries (one read only) were sequenced. Of the 2400 sequences generated, 165 were of poor quality or derived from the cloning vector, leaving 2235 sequences for further analysis.

Sequence Analysis

Of the 2235 sequence fragments, 409 corresponded to 13 previously reported germ-cell-specific genes (142 to Mage, 11 to Ubely, 2 to Usp9y, 44 to Rbmy, 10 to Tuba3/Tuba7, 2 to Stra8, 45 to Ott, 16 to Sycp2, 3 to Sycp1, 3 to Figla, 8 to Sycp3, 21 to Ddx4, and 102 to Dazl). Among the remaining 1826 sequence fragments, each was searched electronically for redundancies and identities to known genes. 98 unique, novel sequence fragments were found that were each recovered at least twice. Each of these 98 sequences was tested for germ cell specificity by RT-PCR on 14 tissues. Of the 98 sequences, 45 were found to be expressed in spermatogonia and wild-type testis, but not in somatic tissues including w^{v}/w^{v} testis, indicating that they are germ cell specific. After full-length cDNA sequences were assembled, these 45 sequence fragments were found to derive from a total of 23 different genes. Of the original set of 2235 sequence fragments, 546 corresponded to these 23 novel genes (8 to Fthl17; 29 to Usp26; 38 to Tktl1; 66 to Tex11; 2 to Tex16; 132 to Taj2q; 57 to Pramel3; 13 to Nxf2; 5 to Tex13; 4 to Pramel1; 3 to Tex17; 2 to Stk31; 6 to Rnh2; 29 to Tex12; 4 to Tex18; 2 to Tex14; 8 to Rnf17; 16 to Piwil2; 36 to Mov10l1; 7 to Tex20; 71 to Tex15; 6 to Tex19; 2 to Tdrd1).

cDNA Cloning

Full-length mouse cDNA sequences were composites derived from subtracted cDNA clones, 5′ and 3′ RACE products, and clones isolated from conventional cDNA libraries that were prepared from adult testes (Clontech, Palo Alto, Calif.; Stratagene, La Jolla, Calif.; and one library of our own construction). Orthologous human sequences were identified by searching GenBank using mouse cDNA sequences. Full-length human cDNA sequences were obtained by screening a cDNA library prepared from adult testes (Clontech).

RH Mapping

Using PCR, genomic DNAs from the 93 cell lines of the mouse T31 radiation hybrid panel (Research Genetics, Huntsville, Ala.) were tested for the presence of each gene (McCarthy, L. C. et al., Genome Res. 7, 1153-1161 (1997). PCR conditions and primer sequences have been deposited at GenBank, where accession numbers are as follows: Figla, G65193; Magea5, G65194; Ddx4, G65195; Ott, G65196; Sycp2, G65197; Sycp3,G65198; Stra8,G65199; Tuba3, G65200; Tuba7, G65201; Fthl17, G65202; Mov10l1,G65203; Nxf2,G65204; Piwil2, G65205; Pramel1, G65206; Pramel3, G65331; RNF17, G65207; Rnh2, G65208; Stk31, G65210; Taf2q, G65211; Tdrd1, G65212; Tex11, G65213; Tex12, G65214; Tex13, G65215; Tex14, G65216; Tex15, G65217; Tex16, G65218; Tex17, G65219; Tex18, G65220; Tex19, G65221; Tex20, G65222; Tktl1, G65223; and Usp26, G65224. Analysis of the results positioned the genes with respect to the radiation hybrid map of the mouse genome constructed at the Whitehead/MIT Center for Genome Research (Van Etten, W. J. et al., Nature Genet. 22, 384-387 (1999) (www-genome.wi.mit.edu/mouse_rh/index.html). Chromosomal mapping data of human genes were retrieved from GenBank and confirmed by RH mapping using the GeneBridge 4 panel (Research Genetics).

Expression Analysis

RT-PCR conditions and primer sequences have been deposited at GenBank, where accession numbers for mouse genes are as follows: Gapd, G65758; Fshr, G65759; Dazl, G65760; Rbmy, G65761; Fthl17, G65778; Mov10l1, G65779; Nxf2, G65780; Piwil2, G65781; Pramel1, G65762; Pramel3, G65782; Rnf17, G65763; Rnh2, G65783; Stk31, G65784; Taf2q, G65785; Tdrd1, G65786; Tex11, G65787; Tex12, G65788; Tex13, G65789; Tex14, G65790; Tex15, G65791; Tex16, G65792; Tex17, G65793; Tex18, G65794; Tex19, G65795; Tex20, G65796; Tktl1, G65797; Usp26, G65798. Accession numbers for human genes are as follows: FTH1, G65764; FTHL17, G65765; MOV10L1, G65766; NXF2, G65767; RNF17, G65799; STK31, G65768; TAF2Q, G65769; TDRD1, G65770; TEX11, G65771; TEX12, G65772; TEX13A, G65773; TEX13B, G65774; TEX14, G65775; TEX15, G65776; USP26, G65777.

### Example 2

Isolation and Cloning of Reproduction-Specific Genes

380 infertile men (217 azoospermia and 163 oligospermia) and 93 fertile males were screened for mutations in two X-linked genes (TAF2Q and TEX 11). The Klondike PCR-based subtraction protocol (Diatchenko, L. et al., Methods Enzymol. 303, 349-80 (1999); Diatchenko, L. et al., Proc. Natl. Acad. Sci. USA 93, 6025-30 (1996) and a modified subtraction protocol (modified by Doug Menke, personal communication) (Lavery, D. J. et al., Proc. Natl. Acad. Sci. USA 94, 6831-6 (1997), Yang M. et al., Anal. Biochem. 237(1):109-14 (1996); Ausubel, F. M. et al., Current Protocols in Molecular Biology (1997)) were used to generate a subtraction cDNA library for each type of spermatogonia. In detail, cDNAs synthesized from mRNAs of infertile males and fertile males' spermatogonia were subtracted against a mixture of cDNAs found in great excess derived from mRNAs of 11 different somatic tissues (heart, brain, lung, liver, skeletal muscle, kidney, spleen, stomach, thymus, skin and w^{v}/w^{v} testis). w^{v}/w^{v} testes are essentially devoid of germ cells (Geissler, E. N. et al., Cell 55, 185-192 (1988)). After subtraction, germ cell-specific genes are expected to be enriched and ubiquitous genes to be removed to a certain degree. The subtractions were successful, as demonstrated by the enrichment of Dazl transcript (germ cell-specific) (Reijo, R., et al., Genomics 35, 346-52 (1996)) and the disappearance of G3PDH transcript (ubiquitous, present in all the tissues). The subtracted cDNAs were directly cloned into a plasmid vector to make a subtracted cDNA library. A library was constructed from infertile men and fertile men. Clones randomly picked from each library were sequenced, using ABI 370 sequencer (ABI, Foster City, Calif.). A total of 2300 sequences was obtained. A combination of different methods was used to obtain full-length cDNA sequences: subtracted DNA sequencing, cDNA library screening of Stratagene and Clontech testis cDNA libraries (Stratagene, La Jolla, Calif. and Clontech, Palo Alto, Calif.), direct RT PCR of testis cDNAs and sequencing, 5′ RACE (rapid amplification of cDNA ends) (Ausubel, F. M. et al., Current Protocols in Molecular Biology (1997)), 3′ RACE, and direct screening and amplification of cDNA library subpools by PCR using one gene-specific primer and one vector-specific primer. To determine the germ cell specificity, RT-PCR assay (reverse transcription polymerase chain reaction) of each clone was performed on a panel of thirteen different tissues (heart, brain, lung, liver, skeletal muscle, kidney, spleen, stomach, thymus, skin and w^{v}/w^{v} testis ) (Ausubel, F. M. et al, Current Protocols in Molecular Biology (1997)). These novel X linked genes are designated FTH1, FTHL17, USP26, TEX 11, TAF2Q, NXF2, TEX13A, TEX13B, STK31, TEX12, TEX14, RNF17, MOV10L1, TEX15 and TDRD1.

The mutations in TEX 11 and TAF2Q were analyzed further. The structure of the gene was assessed, TEX11 BAC's and sequence was screened, primers were chosen spanning each exon. Infertile men were screened and the two genes sequenced. Polymorphism and causality were distinguished by looking at normal male controls, nature of variants, study of maternal relative (linkage), conservation between mouse and human, and splicing in vivo. There were 33 mutations found in TEX11, 12 in exons (4 silent) and 21 in intron. 21 were found only in infertile males (380 males), 1 found only in normal (fertile) males (93 males) and 11 polymorphisms (found in both infertile and normal males). The variants of TEX 11 are depicted in FIG. 108. There were 15 variants found in TAF2Q, 7 in exons and 8 in introns. Of these, 5 were polymorphisms (found in both infertile and normal males), 9 were found only in infertile males, and 1 was found only in normal fertile males. FIG. 112 depicts the variants in TAF2Q.

A combination of different methods was used to obtain full-length CDNA sequences: subtracted DNA sequencing, cDNA library screening of Stratagene and Clontech testis CDNA libraries (Stratagene, La Jolla, Calif. and Clontech, Palo Alto, Calif.), direct RT PCR of testis cDNAs and sequencing, 5′ RACE (rapid amplification of cDNA ends) (Ausubel, F. M. et al, Current Protocols in Molecular Biology (1997)), 3′ RACE, and direct screening and amplification of CDNA library subpools by PCR using one gene-specific primer and one vector-specific primer.

What is claimed is:

1. An isolated reproduction-specific nucleic acid molecule comprising a nucleic acid molecule having a nucleotide sequence selected from the group consisting of
(a) SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and
(b) the complements of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89.

2. A nucleic acid construct comprising an isolated reproduction-specific nucleic acid molecule according to claim 1 operably linked to at least one regulatory sequence.

3. A host cell comprising a nucleic acid construct according to claim 2.

4. An isolated reproduction-specific nucleic acid molecule comprising a portion of a nucleic acid molecule having a nucleotide sequence selected from the group consisting of:
(a) SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and
(b) the complements of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89,
wherein said portion is at least 14 contiguous nucleotides in length.

5. A nucleic acid construct comprising an isolated reproduction-specific nucleic acid molecule according to claim 4 operably linked to at least one regulatory sequence.

6. A host cell comprising a nucleic acid construct according to claim 5.

7. An isolated reproduction-specific nucleic acid molecule comprising a nucleic acid molecule which hybridizes under high stringency hybridization conditions to a nucleic acid molecule having a nucleotide sequence selected from the group consisting of:
(a) SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and
(b) the complements of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89.

8. A nucleic acid construct comprising an isolated reproduction-specific nucleic acid molecule according to claim 7 operably linked to at least one regulatory sequence.

9. A host cell comprising a nucleic acid construct according to claim 8.

10. An isolated reproduction-specific nucleic acid molecule comprising a nucleic acid molecule having a nucleotide sequence which is at least 70% identical to a nucleotide sequence selected from the group consisting of:
(a) SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89; and
(b) the complements of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89.

11 A nucleic acid construct comprising an isolated reproduction-specific nucleic acid molecule according to claim 10 operably linked to at least one regulatory sequence.

12. A host cell comprising a nucleic acid construct according to claim 11.

13. An isolated reproduction-specific nucleic acid molecule which encodes a protein having an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 51, 53, 57, 60, 63, 65, 68, 70, 72, 76, 78, 80, 82, 84, 85, 88, and 90.

14. A nucleic acid construct comprising an isolated reproduction-specific nucleic acid molecule according to claim 13 operably linked to at least one regulatory sequence.

15. A host cell comprising a nucleic acid construct according to claim 14.

16. An isolated variant reproduction-specific nucleic acid molecule comprising a nucleic acid molecule having the nucleic acid sequence of SEQ ID NO: 89 having one or more alterations selected from the group consisting of A320G, T325A, C381T, G400A, A491G, G1282A, C1449A, T2219C, A2250T, T2295C and T2472C.

17. A nucleic acid construct comprising an isolated reproduction-specific nucleic acid molecule according to claim 16 operably linked to at least one regulatory sequence.

18. A host cell comprising a nucleic acid construct according to claim 17.

19. An isolated variant reproduction-specific nucleic acid molecule comprising a nucleic acid molecule having the nucleic acid sequence of SEQ ID NO: 50 having one or more alterations selected from the group consisting of the alterations shown in FIG. 112.

20. A nucleic acid construct comprising an isolated reproduction-specific nucleic acid molecule according to claim 19 operably linked to at least one regulatory sequence.

21. A host cell comprising a nucleic acid construct according to claim 20.

22. An isolated protein comprising an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 51, 53, 57, 60, 63, 65, 68, 70, 72, 76, 78, 80, 82, 84, 85, 88, and 90.

23. An isolated protein comprising a portion of an amino acid sequence selected from the group consisting of SEQ ID NOS: 2, 4, 6, 8, 10, 12, 14, 16, 18, 20, 22, 24, 26, 28, 30, 32, 34, 36, 38, 40, 42, 44, 46, 48, 51, 53, 57, 60, 63, 65, 68, 70, 72, 76, 78, 80, 82, 84, 85, 88, and 90, wherein said portion is at least 7 contiguous amino acids.

24. An isolated protein encoded by a nucleic acid molecule according to claim 1.

25. An isolated protein encoded by a nucleic acid molecule according to claim 4.

26. An isolated protein encoded by a nucleic acid molecule according to claim 7.

27. An isolated protein encoded by a nucleic acid molecule according to claim 10.

28. An isolated protein encoded by a nucleic acid molecule according to claim 13.

29. An isolated protein encoded by a nucleic acid molecule according to claim 16.

30. An isolated protein encoded by a nucleic acid molecule according to claim 19.

31. An antibody which specifically binds a protein according to claim 22.

32. An antibody which specifically binds a protein according to claim 23.

33. An antibody which specifically binds a protein according to claim 24.

34. An antibody which specifically binds a protein according to claim 25.

35. An antibody which specifically binds a protein according to claim 26.

36. An antibody which specifically binds a protein according to claim 27.

37. An antibody which specifically binds a protein according to claim 28.

38. An antibody which specifically binds a protein according to claim 29.

39. An antibody which specifically binds a protein according to claim 30.

40. An isolated protein comprising the amino acid sequence of SEQ ID NO: 90 having one or more alterations selected from the group consisting of W109R, V134I, G164R, N483K and V740A.

41. An antibody which specifically binds a protein according to claim 40.

42. A method of diagnosing infertility associated with alteration of a gene having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89, and whose alteration is associated with infertility, comprising the steps of:
a) obtaining a DNA sample to be assessed;
b) processing the DNA sample such that the DNA is available for hybridization;
c) combining the DNA of step (b) with nucleotide sequences complementary to the altered nucleotide sequence of said gene, whose alteration is associated with infertility, under conditions appropriate for hybridization of the probes with complementary nucleotide sequences in the DNA sample, thereby producing a combination; and
d) detecting hybridization in the combination,
wherein presence of hybridization in the combination is indicative of infertility associated with an alteration of said gene.

43. A method according to claim 42, wherein infertility is a result of reduced sperm count, reduced sperm motility, malformed sperm, or combinations thereof.

44. A method of diagnosing infertility associated with alteration of a gene having a nucleotide sequence selected from the group consisting of SEQ ID NOS: 1, 3, 5, 7, 9, 11, 13, 15, 17, 19, 21, 23, 25, 27, 29, 31, 33, 35, 37, 39, 41, 43, 45, 47, 49, 50, 52, 54, 55, 56, 58, 59, 61, 62, 64, 66, 67, 69, 71, 73, 74, 75, 77, 79, 81, 83, 86, 87 and 89, and whose alteration is associated with infertility, comprising the steps of:
a) obtaining a DNA sample to be assessed;
b) processing the DNA sample such that the DNA is available for hybridization;
c) combining the DNA of step (b) with nucleotide sequences complementary to the nucleotide sequence of said gene, whose alteration is associated with infertility, under conditions appropriate for hybridization of the probes with complementary nucleotide sequences in the DNA sample, thereby producing a combination; and
d) detecting hybridization in the combination,
wherein absence of hybridization in the combination is indicative of infertility associated with an alteration of said gene.

45. A method according to claim 42, wherein infertility is a result of reduced sperm count, reduced sperm motility, malformed sperm, or combinations thereof.
